(19) United States
(12) Patent Application Publication
(10) Pub. No.: US 2023/0042665 A1
(43) Pub. Date: Feb. 9, 2023

(54) PHYSICAL MIX HA-COLLAGEN DERMAL FILLERS

(71) Applicant: ALLERGAN, INC., Irvine, CA (US)

(72) Inventors: Iossif STREHIN, Irvine, CA (US); Xiaojie YU, Orange, CA (US); Darin J. MESSINA, Laguna Niguel, CA (US); Julie MORELL, Annecy (FR); Florent DURIEUX, Alex (FR); Jean-Xavier ROCA MARTINEZ, Annecy (FR)

(21) Appl. No.: 17/789,159

(22) PCT Filed: Dec. 28, 2020
(86) PCT No.: PCT/US2020/067232

§ 371 Date: Jun. 24, 2022

Related U.S. Application Data
(60) Provisional application No. 62/953,925, filed on Dec. 26, 2019.

(51) Int. Cl. A61K 8/73; A61K 8/65; A61L 27/52

(52) U.S. Cl. CPC A61K 8/735; A61K 8/65; A61L 27/52; A61L 2400/06

(57) ABSTRACT

The disclosure relates to compositions comprising crosslinked hyaluronic acid physically mixed with collagen. Also contemplated are methods of improving an aesthetic quality of an anatomic feature of a human being.

### CROSS REFERENCE TO RELATED APPLICATIONS

This application claims the benefit of priority of U.S. Patent Application No. 62/953,925, filed Dec. 26, 2019, which is incorporated by reference herein in its entirety.

### FIELD

This disclosure relates to compositions and methods of making compositions comprising hyaluronic acid mixed with collagen for use as a filler. Additional methods include methods of treating volume deficits within tissue.

### BACKGROUND

The embodiments described herein are related to dermal filler compositions, and more specifically dermal filler compositions comprising hyaluronic acid that are physically mixed with collagen.

The aging of skin occurs over time and can be exacerbated by a variety of factors such as sun exposure (UVA and UVB rays), genetics and bad personal habits such as smoking, drinking and poor diet, for example. As skin ages, the skin may feel and appear rougher and the skin may also start to thin and become less elastic.

Hyaluronan, also known as hyaluronic acid (HA) is a non-sulfated glycosaminoglycan that is distributed widely throughout the human body in connective, epithelial, and neural tissues. Hyaluronan is abundant in the different layers of the skin, where it functions to ensure good hydration, assist in the organization of the extracellular matrix, act as a space filler; and participate in tissue repair mechanisms. However, with age, the quantity of hyaluronan, collagen, elastin, and other matrix polymers present in the skin decreases. For example, repeated exposure to ultra violet light from the sun causes dermal cells to both decrease their production of hyaluronan as well as increase the rate of its degradation. This hyaluronan loss contributes to various skin conditions such as, skin dryness, wrinkles, imperfections, defects, and reduced skin thickness.

The body also has endogenous collagen which is found in tendons, muscles, bones, skin, and ligaments. However, over time collagen components in the body may be lost due to exposure to ultraviolet light, tobacco, alcohol, and the aging process. Decreased collagen in the skin can result in the loss of skin elasticity, the reduction in skin thickness, formation of wrinkles, and sagging.

As such, there is an increasing interest in surgical and nonsurgical treatments to address aging. Nonsurgical treatments include botulinum toxin injections, and injections of soft-tissue fillers, which are the most frequently performed procedures for facial rejuvenation.

Dermal fillers have been used for treating volume deficiency, smoothing the appearance of wrinkles, correction of facial asymmetry, as well as contouring the face and other parts of the body. The results may last several months to two years, depending on the location of the filler injection and the patient; however, the effect of the filler is temporary and it requires subsequent treatment to maintain the desired aesthetic effect.

Hyaluronic acid (HA) based fillers were developed almost twenty years ago and treatment with such fillers is one of the most commonly performed procedure in cosmetic dermatology practice. A major advantage of HA fillers is that the HA produced by biotechnological methods can be chemically identical to HA that is naturally produced by the human body, thus it rarely gives rise to an allergic response. HA is susceptible to enzymatic degradation in the tissues due to endogenous enzymes and clearance by the body, and thus, HA can be crosslinked or otherwise modified to increase the residence time of the filler. Additionally, because HA is susceptible to enzymatic degradation by hyaluronidase enzymes, HA fillers can be quickly degraded, and the filling effect reversed by treatment with exogenous hyaluronidase in the case of an adverse event. Thus, HA fillers are an attractive choice for many patients as the correction from HA treatment is long lasting and HA fillers have a good safety profile.

Aside from the many advantages, HA fillers also have several disadvantages. One such disadvantage is the Tyndall effect, a bluish hue which can be visible at the site of certain superficial HA filler injections. Swelling at the injection site can also occur since HA is hydrophilic and will absorb water. It is also noted that with a greater degree of crosslinking, HA may have a higher risk of inflammation and granuloma formation.

Available prior to the appearance of HA fillers on the market, collagen fillers were introduced into the market in the 1980s. Collagen has several favorable properties as a filler material. For example, collagen fillers are opaque and therefore injection of these materials under the skin does not result in the Tyndall effect as seen with the HA fillers. Additionally, collagen can act as scaffold and support tissue ingrowth into the filler material. However, collagen fillers are not without drawbacks. High collagen concentrations in the filler formulation are required to achieve the desired filling effect and despite these high collagen concentrations, the volumizing and wrinkle filling capacity of the materials is lower than those of HA fillers. Additionally, the duration of wrinkle correction has been shown to be only 3-6 months and these fillers are not reversible since there is no enzyme treatment available to degrade collagen materials.

As such, a need for a dermal filler that provides the advantages of both the HA and the collagen filler are desired. As described in the embodiments herein, is a composition that provides lift and volume upon injection while delivering the benefits of collagen and HA fillers.

### SUMMARY

Accordingly, new dermal filler compositions, as well as methods of making same, are provided. Some embodiments include homogeneous hydrogel compositions prepared from hyaluronic acid and collagen. Some embodiments, also include hydrogel compositions containing particles of HA and/or collagen, that is evenly mixed throughout. Some embodiments may also include partially homogeneous or heterogeneous products containing particles of HA or collagen, such as collagen fibrils. These compositions may be prepared by a method comprising crosslinking hyaluronic acid, thereby producing a crosslinked hyaluronic acid and then mixing the crosslinked hyaluronic acid with collagen.

In a first aspect, a method of preparing a macromolecular matrix is provided. The method comprises providing a crosslinked hyaluronic acid, providing collagen, and physically mixing the collagen into the crosslinked hyaluronic acid, wherein the collagen is mixed homogeneously throughout the crosslinked hyaluronic acid, thereby forming the macromolecular matrix, wherein the macromolecular matrix comprises crosslinked hyaluronic acid and physically mixed in collagen.

In some embodiments of any one of each or any of the above- or below-mentioned embodiments, the collagen is provided in a soluble state as a solution.

In some embodiments of any one of each or any of the above- or below-mentioned embodiments, the collagen is provided as a solution, wherein the collagen solution comprises a pH of about 2.0, about 2.5, about 3.0, about 3.5, about 4.0, about 4.5, about 5.0, 5.5, about 6.0, about 6.5, about 7.0 or any pH within a range defined by any two aforementioned values, and wherein the collagen is soluble at the acidic pH. In some embodiments of any one of each or any of the above- or below-mentioned embodiments, the collagen is provided as a solution in an acidic pH, wherein the collagen solution comprises a pH of about 2.0, about 2.5, about 3.0, about 3.5, about 4.0, about 4.5, about 5.0, about 5.5 or any pH within a range defined by any two aforementioned values, and wherein the collagen is soluble at the acidic pH. In some embodiments of any one of each or any of the above- or below-mentioned embodiments, the collagen is provided as a solution at a neutral pH.

In some embodiments of any one of each or any of the above- or below-mentioned embodiments, the collagen and the crosslinked hyaluronic acid are physically mixed with a buffer. In some embodiments of any one of each or any of the above- or below-mentioned embodiments, the buffer comprises PBS.

In some embodiments of any one of each or any of the above- or below-mentioned embodiments, method further comprises neutralizing the macromolecular matrix to a pH of about 7, after mixing the collagen homogeneously throughout the crosslinked hyaluronic acid.

In some embodiments of any one of each or any of the above- or below-mentioned embodiments, the method further comprises neutralizing the collagen to a pH of about 7, prior to physically mixing the collagen with the crosslinked hyaluronic acid. In some embodiments of any one of each or any of the above- or below-mentioned embodiments, neutralizing the collagen prior to physically mixing the collagen into the crosslinked hyaluronic acid causes the collagen to precipitate into fibrils or particles of collagen, wherein the fibrils or particles of collagen are further mixed into the crosslinked hyaluronic acid, wherein the fibrils or particles are mixed in homogeneously throughout the crosslinked hyaluronic acid.

In some embodiments of any one of each or any of the above- or below-mentioned embodiments, the collagen is provided as fibrillated collagen or collagen fibers. In some embodiments of any one of each or any of the above- or below-mentioned embodiments, wherein the collagen is provided as fibrillated collagen, and wherein the collagen was prepared at a neutral or basic pH thereby producing fibrillated collagen of collagen fibers.

In some embodiments of any one of each or any of the above- or below-mentioned embodiments, the collagen was prepared with at least one salt to obtain fibrillated collagen or collagen fibers. In some embodiments of any one of each or any of the above- or below-mentioned embodiments, the at least one salt comprises a concentration of about 20 mM, about 50 mM, about 100 mM, about 150 mM, about 200 mM, about 250 mM, about 300 mM, about 350 mM, about 400 mM, about 450 mM, or about 500 mM, or any concentration in between a range defined by any two aforementioned values. In some embodiments of any one of each or any of the above- or below-mentioned embodiments, the at least one salt comprises an anion wherein the anion comprises H₂PO⁴⁻, SO₄²⁻, Cl⁻ or SCN⁻. In some embodiments of any one of each or any of the above- or below-mentioned embodiments, the at least one salt comprises NaCl, Na₂SO₄, or Li₂SO₄.

In some embodiments of any one of each or any of the above- or below-mentioned embodiments, the collagen is mixed homogeneously throughout the macromolecular matrix.

In some embodiments of any one of each or any of the above- or below-mentioned embodiments, the crosslinked hyaluronic acid comprises hyaluronic acid components comprising an average molecular weight of about 10,000 Daltons, about 20,000 Daltons, about 30,000 Daltons, about 40,000 Daltons, about 50,000 Daltons, about 60,000 Daltons, about 70,000 Daltons, about 80,000 Daltons, about 90,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,100,000 Daltons, about 1,200,000 Daltons, about 1,300,000 Daltons, about 1,400,000 Daltons, about 1,500,000 Daltons, about 1,600,000 Daltons, about 1,700,000 Daltons, about 1,800,000 Daltons, about 1,900,000 Daltons, about 2,000,000 Daltons, about 2,100,000 Daltons, about 2,200,000 Daltons, about 2,300,000 Daltons, about 2,400,000 Daltons, about 2,500,000 Daltons, about 2,600,000 Daltons, about 2,700,000 Daltons, about 2,800,000 Daltons, about 2,900,000 Daltons, about 3,000,000 Daltons, about 3,100,000 Daltons, about 3,200,000 Daltons, about 3,300,000 Daltons, about 3,400,000 Daltons, about 3,500,000 Daltons, about 3,600,000 Daltons, about 3,700,000 Daltons, about 3,800,000 Daltons, about 3,900,000 Daltons, about 4,000,000 Daltons, about 4,100,000 Daltons, about 4,200,000 Daltons, about 4,300,000 Daltons, about 4,400,000 Daltons, about 4,500,000 Daltons, about 4,600,000 Daltons, about 4,700,000 Daltons, about 4,800,000 Daltons, about 4,900,000 Daltons, about 5,000,000 Daltons, about 5,100,000 Daltons, about 5,200,000 Daltons, about 5,300,000 Daltons, about 5,400,000 Daltons, about 5,500,000 Daltons, about 5,600,000 Daltons, about 5,700,000 Daltons, about 5,800,000 Daltons, about 5,900,000 Daltons, about 6,000,000 Daltons, about 6,100,000 Daltons, about 6,200,000 Daltons, about 6,300,000 Daltons, about 6,400,000 Daltons, about 6,500,000 Daltons, about 6,600,000 Daltons, about 6,700,000 Daltons, about 6,800,000 Daltons, about 6,900,000 Daltons, about 7,000,000 Daltons, about 7,100,000 Daltons, about 7,200,000 Daltons, about 7,300,000 Daltons, about 7,400,000 Daltons, about 7,500,000 Daltons, about 7,600,000 Daltons, about 7,700,000 Daltons, about 7,800,000 Daltons, about 7,900,000 Daltons, about 8,000,000 Daltons, about 8,100,000 Daltons, about 8,200,000 Daltons, about 8,300,000 Daltons, about 8,400,000 Daltons, about 8,500,000 Daltons, about 8,600,000 Daltons, about 8,700,000 Daltons, about 8,800,000 Daltons, about 8,900,000 Daltons, about 9,000,000 Daltons, about 9,100,000 Daltons, about 9,200,000 Daltons, about 9,300,000 Daltons, about 9,400,000 Daltons, about 9,500,000 Daltons, about 9,600,000 Daltons, about 9,700,000 Daltons, about 9,800,000 Daltons, about 9,900,000 Daltons or about 10,000,000 Daltons or any molecular weight in between a range defined by any two aforementioned values. In some embodiments of any one of each or any of the above- or below-mentioned embodiments, the crosslinked hyaluronic acid comprises a mixture of hyaluronic acid components comprising at least two different molecular weights.

In some embodiments of any one of each or any of the above- or below-mentioned embodiments, the method further comprises adding lidocaine to the macromolecular matrix. In some embodiments of any one of each or any of the above- or below-mentioned embodiments, the lidocaine is at a concentration in between a range of 0.15% (w/w) to 0.45% (w/w) in the matrix. In some embodiments of any one of each or any of the above- or below-mentioned embodiments, the lidocaine is at a concentration in between a range of 0.27% (w/w) to 0.33% (w/w) in the matrix. In some embodiments of any one of each or any of the above- or below-mentioned embodiments, the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w), about 0.39% (w/w), about 0.41% (w/w), about 0.43% (w/w), or about 0.45% (w/w) of the matrix, or any concentration in between a range defined by any two aforementioned values. In some embodiments of any one of each or any of the above- or below-mentioned embodiments, the lidocaine is at a concentration of about 0.3% (w/w) in the matrix.

In some embodiments of any one of each or any of the above- or below-mentioned embodiments, the method further comprises adding un-crosslinked HA to the matrix. In some embodiments of any one of each or any of the above- or below-mentioned embodiments, the un-crosslinked HA comprises a concentration of up to about 5% (w/w) within the matrix. In some embodiments of any one of each or any of the above- or below-mentioned embodiments, the un-crosslinked HA comprises a concentration of about 0% (w/w), about 1% (w/w), about 2% (w/w), about 3% (w/w), about 4% (w/w), about 5% (w/w) in the matrix or any concentration in between a range defined by any two aforementioned values. In some embodiments of any one of each or any of the above- or below-mentioned embodiments, the un-crosslinked HA comprises a concentration of about 1% (w/w) in the matrix. In some embodiments of any one of each or any of the above- or below-mentioned embodiments, the un-crosslinked HA comprises a concentration of 2% (w/w) in the matrix. In some embodiments of any one of each or any of the above- or below-mentioned embodiments, the un-crosslinked HA comprises a concentration of about 5% (w/w) in the matrix. In some embodiments of any one of each or any of the above- or below-mentioned embodiments, the un-crosslinked HA, improves the extrudability of the matrix.

In some embodiments of any one of each or any of the above- or below-mentioned embodiments, the macromolecular matrix has enhanced extrudability through a needle, wherein the needle comprises a gauge size of 27G, 28G, 29G, 30G, 31G or 32G.

In some embodiments of any one of each or any of the above- or below-mentioned embodiments, the macromolecular matrix is stable for at least 6 months. In some embodiments of any one of each or any of the above- or below-mentioned embodiments, the macromolecular matrix is stable for about 6, about 9, about 12, about 15, about 18, about 21, about 24, about 27, about 30, about 33 or about 36 months or any amount of time in between a range defined by any two aforementioned values. In some embodiments of any one of each or any of the above- or below-mentioned embodiments, the macromolecular matrix is stable at a temperature between about 4° C. to about 25° C. In some embodiments of any one of each or any of the above- or below-mentioned embodiments, the macromolecular matrix is stable at a temperature of about 4° C., about 5° C., about 10° C., about 15° C., about 20° C., about 25° C. or any temperature in between a range defined by any two aforementioned values. In some embodiments of any one of each or any of the above- or below-mentioned embodiments, the macromolecular matrix is stable at 4° C. In some embodiments of any one of each or any of the above- or below-mentioned embodiments, the macromolecular matrix is stable at about 25° C.

In some embodiments of any one of each or any of the above- or below-mentioned embodiments, the macromolecular matrix has minimal degradation at about 3, about 6, about 9, about 12, about 15, about 18, about 21, about 24, about 27, about 30, about 33, about 36 months or any amount of time in between a range defined by any two aforementioned values.

In a second aspect, a method of preparing a macromolecular matrix is provided. The method comprises dissolving hyaluronic acid in an aqueous solution to form an aqueous pre-reaction solution and preparing a second solution wherein the second solution comprises a water soluble carbodiimide; and an N-hydroxysuccinimide or an N-hydroxysulfosuccinimide; or a 1,4-butanediol diglycidyl ether (BDDE) in a solution of sodium hyaluronate in the presence of sodium hydroxide; and adding the second solution to the aqueous pre-reaction solution to form a crosslinking reaction mixture; crosslinking the hyaluronic acid, wherein the crosslinking reaction mixture reacts by crosslinking the hyaluronic acid , thereby forming the crosslinked hyaluronic acid; and providing collagen, and physically mixing the collagen into the crosslinked hyaluronic acid, thereby producing a macromolecular matrix comprising crosslinked hyaluronic acid and collagen, wherein the collagen is mixed homogeneously throughout the crosslinked hyaluronic acid. In some embodiments of any one of each or any of the above- or below-mentioned embodiments, the second solution comprises the water soluble carbodiimide and the N-hydroxysuccinimide or the N- hydroxysulfosuccinimide, wherein the hyaluronic acid is crosslinked with a naturally occurring amine, thereby forming the crosslinked hyaluronic acid. In some embodiments of any one of each or any of the above- or below-mentioned embodiments, the second solution comprises the water soluble carbodiimide and the N-hydroxysuccinimide or the N- hydroxysulfosuccinimide and wherein the crosslinking is performed in the presence of MES. In some embodiments of any one of each or any of the above- or below-mentioned embodiments, the second solution comprises the 1,4-butanediol diglycidyl ether (BDDE) in a solution of sodium hyaluronate in the presence of sodium hydroxide, and wherein the BDDE is used to cross-link the hyaluronic acid using epoxide chemistry. In some embodiments of any one of each or any of the above- or below-mentioned embodiments, the physically mixing step is performed in the presence of a buffer. In some embodiments of any one of each or any of the above- or below-mentioned embodiments, the buffer comprises PBS. In some embodiments of any one of each or any of the above- or below-mentioned embodiments, the collagen is provided as a collagen solution, wherein the collagen solution comprises a pH of about 2.0, about 2.5, about 3.0, about 3.5, about 4.0, about 4.5, about 5.0, about 5.5, about 6.0, about 6.5, or about 7.0 or any pH within a range defined by any two aforementioned values, and wherein the collagen is soluble at an acidic pH. In some embodiments of any one of each or any of the above- or below-mentioned embodiments, the collagen is provided as a solution in an acidic pH, wherein the collagen solution comprises a pH of about 2.0, about 2.5, about 3.0, about 3.5, about 4.0, about 4.5, about 5.0, about 5.5 or any pH within a range defined by any two aforementioned values, and wherein the collagen is soluble at the acidic pH. In some embodiments of any one of each or any of the above- or below-mentioned embodiments, the collagen is provided as a collagen solution, wherein the collagen solution comprises a pH of about 2.0, about 2.5, about 3.0, about 3.5, about 4.0, about 4.5, about 5.0, about 5.5, about 6.0, about 6.5, or about 7.0 or any pH within a range defined by any two aforementioned values, and wherein the collagen is soluble at an acidic pH.

In some embodiments of any one of each or any of the above- or below-mentioned embodiments, the collagen is provided at an acidic to neutral pH.

In some embodiments of any one of each or any of the above- or below-mentioned embodiments, the method further comprises neutralizing the macromolecular matrix to a pH of about 7, after mixing the collagen homogeneously throughout the crosslinked hyaluronic acid.

In some embodiments of any one of each or any of the above- or below-mentioned embodiments, the method further comprises neutralizing the macromolecular matrix to a pH of about 7, after mixing the collagen homogeneously throughout the crosslinked hyaluronic acid.

In some embodiments of any one of each or any of the above- or below-mentioned embodiments, the collagen is provided in a solution of about pH 5 to pH 7 for pre-fibrillation. In some embodiments of any one of each or any of the above- or below-mentioned embodiments, the collagen is provided in a solution comprising a pH of about 5.0, about 5.5, about 6.0, about 6.5, or about 7, or any pH in between a range defined by any two aforementioned values for pre-fibrillation.

In some embodiments of any one of each or any of the above- or below-mentioned embodiments, the method further comprises neutralizing the macromolecular matrix to a pH of about 7, after mixing the collagen homogeneously throughout the crosslinked hyaluronic acid.

In some embodiments of any one of each or any of the above- or below-mentioned embodiments, the method further comprises comprising neutralizing the collagen to a pH of about 7, prior to physically mixing the collagen with the crosslinked hyaluronic acid, wherein neutralizing the collagen prior to physically mixing the collagen into the crosslinked hyaluronic acid causes the collagen to precipitate into fibrils or particles of collagen, wherein the fibrils or particles of collagen are further mixed into the crosslinked hyaluronic acid, wherein the fibrils or particles are mixed in homogeneously throughout the crosslinked hyaluronic acid.

In some embodiments of any one of each or any of the above- or below-mentioned embodiments, the collagen is provided as fibrillated collagen or collagen fibers.

In some embodiments of any one of each or any of the above- or below-mentioned embodiments, the collagen was prepared at a basic pH thereby producing the fibrillated collagen or collagen fibers.

In some embodiments of any one of each or any of the above- or below-mentioned embodiments, the collagen was prepared with at least one salt to obtain fibrillated collagen or collagen fibers.

In some embodiments of any one of each or any of the above- or below-mentioned embodiments, the collagen was prepared with at least one salt to obtain fibrillated collagen or collagen fibers.

In some embodiments of any one of each or any of the above- or below-mentioned embodiments, the at least one salt comprises a concentration of about 20 mM, about 50 mM, about 100 mM, about 150 mM, about 200 mM, about 250 mM, about 300 mM, about 350 mM, about 400 mM, about 450 mM, or about 500 mM, or any concentration in between a range defined by any two aforementioned values.

In some embodiments of any one of each or any of the above- or below-mentioned embodiments, the at least one salt comprises an anion wherein the anion comprises H₂PO⁴⁻, SO₄²⁻, Cl⁻ or SCN⁻. In some embodiments of any one of each or any of the above- or below-mentioned embodiments, the at least one salt comprises NaCl, Na₂SO₄, or Li₂SO₄.

In some embodiments of any one of each or any of the above- or below-mentioned embodiments, the hyaluronic acid comprises a mixture of hyaluronic acid components comprising at least two different molecular weights.

In some embodiments of any one of each or any of the above- or below-mentioned embodiments, the dissolving step further comprises dissolving both hyaluronic acid and collagen in an aqueous solution to form an aqueous pre-reaction solution comprising both hyaluronic acid and collagen and wherein the crosslinking reaction reacts by crosslinking both the hyaluronic acid and collagen into an HA-Collagen conjugated gel, and wherein the mixing step comprises physically mixing the HA-Collagen conjugated gel with additional collagen, wherein the collagen is in a soluble state and wherein the collagen is in a solution, wherein the solution is in an acidic pH, thereby producing a macromolecular matrix comprising HA-Collagen conjugated gel with physically mixed in collagen.

In some embodiments of any one of each or any of the above- or below-mentioned embodiments, collagen fibrils or collagen precipitates are formed after the mixing step with collagen, and wherein the collagen fibrils or collagen precipitates are mixed homogenously within the crosslinked hyaluronic acid. In some embodiments of any one of each or any of the above- or below-mentioned embodiments, collagen fibrils or collagen precipitates are formed during the mixing step with collagen, and wherein the collagen fibrils or collagen precipitates are mixed homogenously within the crosslinked hyaluronic acid.

In some embodiments of any one of each or any of the above- or below-mentioned embodiments, the method further comprises adjusting an osmolarity of the macromolecular matrix after the collagen is mixed throughout the crosslinked hyaluronic acid.

In some embodiments of any one of each or any of the above- or below-mentioned embodiments, the mixing step is performed at room temperature. In some embodiments of any one of each or any of the above- or below-mentioned embodiments, the mixing step is performed at about 4° C. In some embodiments of any one of each or any of the above- or below-mentioned embodiments, the method is performed at about 4° C.

In some embodiments of any one of each or any of the above- or below-mentioned embodiments, the method further comprises purifying the crosslinked hyaluronic acid or HA-Collagen conjugated gel, wherein the purifying is performed prior to the mixing step with the collagen. In some embodiments of any one of each or any of the above- or below-mentioned embodiments, the purifying is performed using dialysis purification. In some embodiments of any one of each or any of the above- or below-mentioned embodiments, the pH of the crosslinking reaction mixture is adjusted after the crosslinking is complete, wherein adjusting the pH is performed prior to the purifying step, and wherein the pH is adjusted to about 7.0, about 7.2, about 7.4 about 7.6 or any pH in between a range defined by any two aforementioned values. In some embodiments of any one of each or any of the above- or below-mentioned embodiments, the purifying is performed at a range between about 2° C. to about 8° C. In some embodiments of any one of each or any of the above- or below-mentioned embodiments, the purifying is performed at about 2° C., about 4° C., about 6° C., or about 8° C., or any temperature in between a range defined by any two aforementioned values.

In some embodiments of any one of each or any of the above- or below-mentioned embodiments, the method further comprises sterilizing the crosslinked hyaluronic acid or HA-Collagen conjugated gel prior to mixing with collagen, wherein the sterilizing step is performed after the purifying step. In some embodiments of any one of each or any of the above- or below-mentioned embodiments, the sterilizing step comprises transferring the crosslinked hyaluronic acid or HA-Collagen conjugated gel into a container, for sterilization; and sterilizing the crosslinked hyaluronic acid or HA-Collagen conjugated gel. In some embodiments of any one of each or any of the above- or below-mentioned embodiments, the container is a syringe. In some embodiments of any one of each or any of the above- or below-mentioned embodiments, the sterilizing is performed by heat (dry heat, steam heat, moist heat sterilization) radiation (i.e. non-ionizing, UV), ionizing (particulate (e.g. beta rays, gamma rays, x-rays), electromagnetic (e.g. e-beam), filtration) or terminal sterilization.

In some embodiments of any one of each or any of the above- or below-mentioned embodiments, the method further comprises dialyzing the crosslinked macromolecular matrix or HA-Collagen conjugated gel, wherein dialysis is performed through a membrane having a molecular weight cutoff of in a range between about 1000 Daltons to about 100,000 Daltons, and wherein the dialyzing is performed prior to sterilization. In some embodiments of any one of each or any of the above- or below-mentioned embodiments, the dialysis is performed in a buffer. In some embodiments of any one of each or any of the above- or below-mentioned embodiments, the dialysis is performed in a phosphate buffered saline or a sodium phosphate buffer. In some embodiments of any one of each or any of the above- or below-mentioned embodiments, the buffer further comprises NaCl, and/or KCl.

In some embodiments of any one of each or any of the above- or below-mentioned embodiments, the method further comprises raising the pH of the crosslinked hyaluronic acid or HA-Collagen conjugated gel to a neutral pH after the crosslinking reaction is complete, wherein the raising the pH is performed prior to a sterilizing step. In some embodiments of any one of each or any of the above- or below-mentioned embodiments, the method further comprises raising the pH of the crosslinked hyaluronic acid or HA-Collagen conjugated gel to about 7.0, about 7.2 or about 7.4, or any pH in between a range defined by any two aforementioned values, after the crosslinking reaction is complete, wherein the raising the pH is performed prior to a sterilizing step.

In some embodiments of any one of each or any of the above- or below-mentioned embodiments, the second solution comprises the water soluble carbodiimide and the N-hydroxysuccinimide or an N-hydroxysulfosuccinimide, and wherein the adding and crosslinking step is performed at a temperature between about 2° C. and about 22° C. In some embodiments of any one of each or any of the above- or below-mentioned embodiments, the adding and crosslinking step is performed at a temperature of about 2° C., about 4° C., about 6° C., about 8° C., about 10° C., about 12° C., about 14° C., about 16° C., about 18° C., about 20° C., about 22° C., or any temperature in between a range defined by any two aforementioned values.

In some embodiments of any one of each or any of the above- or below-mentioned embodiments, the crosslinking is performed at about 22° C. In some embodiments of any one of each or any of the above- or below-mentioned embodiments, the crosslinking is performed at about 4° C.

In some embodiments of any one of each or any of the above- or below-mentioned embodiments, the second solution comprises the 1,4-butanediol diglycidyl ether (BDDE) in the solution of sodium hyaluronate in the presence of sodium hydroxide, and wherein the adding and crosslinking step is performed between about 45° C. and about 75° C. In some embodiments of any one of each or any of the above- or below-mentioned embodiments, the second solution comprises the 1,4-butanediol diglycidyl ether (BDDE) in the solution of sodium hyaluronate in the presence of sodium hydroxide, and wherein the adding and crosslinking step is performed at a temperature of about 45° C., about 50° C., about 55° C., about 60° C., about 65° C., about 70° C., about 75° C., or any temperature in between a range defined by any two aforementioned values. In some embodiments of any one of each or any of the above- or below-mentioned embodiments, the second solution comprises the 1,4-butanediol diglycidyl ether (BDDE) in the solution of sodium hyaluronate in the presence of sodium hydroxide, and wherein the adding and crosslinking step is performed at a temperature of about 50° C.

In some embodiments of any one of each or any of the above- or below-mentioned embodiments, the method further comprises stopping the crosslinking step, wherein the stopping step comprises adding a base to the crosslinking reaction mixture to a pH of between about 8 to about 10 for at least about 10 minutes and then adding an acidic solution to the crosslinking mixture until a pH of about 7 is reached.

In some embodiments of any one of each or any of the above- or below-mentioned embodiments, the crosslinking reaction mixture comprises a pH between about 4.0 or about 10.0. In some embodiments of any one of each or any of the above- or below-mentioned embodiments, the pH of the crosslinking reaction mixture is between about 4.0 or about 6.0.

In some embodiments of any one of each or any of the above- or below-mentioned embodiments, the second solution comprises 1,4-butanediol diglycidyl ether (BDDE) in a solution of sodium hyaluronate in the presence of sodium hydroxide, and wherein the crosslinking is performed under alkaline conditions.

In some embodiments of any one of each or any of the above- or below-mentioned embodiments, the pre-reaction solution comprises a salt, wherein the salt comprises a concentration of about 50 mM, about 75 mM, about 100 mM, about 125 mM, about 150 mM, about 175 mM, about 200 mM, about 225 mM, about 250 mM, about 275 mM, about 300 mM, 325 mM, about 350 mM, about 375 mM, about 400 mM, about 425 mM, about 450 mM, about 475 mM, about 500 mM or any concentration in a range defined by any two aforementioned values.

In some embodiments of any one of each or any of the above- or below-mentioned embodiments, the water soluble carbodiimide is 1-ethyl-3-(3-dimethylaminopropyl)carbodiimide and wherein the water soluble carbodiimide is at a concentration of about 20 mM to about 300 mM in the crosslinking reaction mixture. In some embodiments of any one of each or any of the above- or below-mentioned embodiments, the concentration of the water soluble carbodiimide is 1-ethyl-3-(3-dimethylaminopropyl)carbodiimide is at a concentration of about 20 mM, about 40 mM, about 60 mM, about 80 mM, about 100 mM, about 120 mM, about 140 mM, about 160 mM, about 180 mM, about 200 mM, about 220 mM, about 240 mM, about 260 mM, about 280 mM, about 300 or any concentration in between a range defined by any two aforementioned values. In some embodiments of any one of each or any of the above- or below-mentioned embodiments, the water soluble carbodiimide and hyaluronic acid is at a mole to mole ratio of water soluble carbodiimide: hyaluronic acid repeat unit between about 0.3 to about 3.0. In some embodiments of any one of each or any of the above- or below-mentioned embodiments, the water soluble carbodiimide and hyaluronic acid is at a mole to mole ratio of water soluble carbodiimide: hyaluronic acid repeat unit of about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9 or about 3.0 or any mole to mole ratio in between a range defined by any two aforementioned values.

In some embodiments of any one of each or any of the above- or below-mentioned embodiments, the lysine and hyaluronic acid are at a mole:mole (lysine:HA repeat unit) ratio between about 0.01 to about 0.6. In some embodiments of any one of each or any of the above- or below-mentioned embodiments, the lysine and hyaluronic acid are at a mole:mole (lysine:HA repeat unit) ratio of about 0.01, about 0.02, about 0.03, about 0.04, about 0.05, about 0.06, about 0.07, about 0.08, about 0.09, about 0.10, about 0.11, about 0.12, about 0.13, about 0.14, about 0.15, about 0.16, about 0.17, about 0.18, about 0.19, about 0.2, about 0.21, about 0.22, about 0.23, about 0.24, about 0.25, about 0.26, about 0.27, about 0.28, about 0.29, about 0.3, about 0.31, about 0.32, about 0.33, about 0.34, about 0.35, about 0.36, about 0.37, about 0.38, about 0.39, about 0.4, about 0.41, about 0.42, about 0.43, about 0.44, about 0.45, about 0.46, about 0.47, about 0.48, about 0.49, about 0.5, about 0.51, about 0.52, about 0.53, about 0.54, about 0.55, about 0.56, about 0.57, about 0.58, about 0.59, about 0.6 or any mole to mole ratio in between a range defined by any two aforementioned values.

In some embodiments of any one of each or any of the above- or below-mentioned embodiments, the hyaluronic acid in the pre-reaction solution hydrates for at least 60 minutes prior to the adding the second solution. In some embodiments of any one of each or any of the above- or below-mentioned embodiments, the crosslinking reaction mixture is performed for about 4 hours to about 24 hours.

In some embodiments of any one of each or any of the above- or below-mentioned embodiments, the method further comprises adding lidocaine to the macromolecular matrix. In some embodiments of any one of each or any of the above- or below-mentioned embodiments, the lidocaine is at a concentration in between a range of about 0.15% (w/w) to about 0.45% (w/w) in the matrix. In some embodiments of any one of each or any of the above- or below-mentioned embodiments, the lidocaine is at a concentration in between a range of about 0.27% (w/w) to about 0.33% (w/w) in the matrix. In some embodiments of any one of each or any of the above- or below-mentioned embodiments, the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w), about 0.39% (w/w), about 0.41% (w/w), about 0.43% (w/w), or about 0.45% (w/w) of the matrix, or any concentration in between a range defined by any two aforementioned values. In some embodiments of any one of each or any of the above- or below-mentioned embodiments, the lidocaine is at a concentration of about 0.3% (w/w) in the matrix.

In some embodiments of any one of each or any of the above- or below-mentioned embodiments, the method further comprises adding un-crosslinked HA to the matrix. In some embodiments of any one of each or any of the above- or below-mentioned embodiments, the un-crosslinked HA comprises a concentration of up to about 5% (w/w) within the matrix. In some embodiments of any one of each or any of the above- or below-mentioned embodiments, the un-crosslinked HA comprises a concentration of about 0% (w/w), about 1% (w/w), about 2% (w/w), about 3% (w/w), about 4% (w/w), about 5% (w/w) in the matrix or any concentration in between a range defined by any two aforementioned values. In some embodiments of any one of each or any of the above- or below-mentioned embodiments, the un-crosslinked HA comprises a concentration of about 1% (w/w) in the matrix. In some embodiments of any one of each or any of the above- or below-mentioned embodiments, the un-crosslinked HA comprises a concentration of about 2% (w/w) in the matrix. In some embodiments of any one of each or any of the above- or below-mentioned embodiments, the un-crosslinked HA comprises a concentration of about 5% (w/w) in the matrix. In some embodiments of any one of each or any of the above- or below-mentioned embodiments, the un-crosslinked HA, improves the extrudability of the matrix.

In some embodiments of any one of each or any of the above- or below-mentioned embodiments, the method comprises a neutralizing step performed after forming the macromolecular matrix comprising the crosslinked hyaluronic acid and collagen or after forming the HA-Collagen conjugated gel with physically mixed in collagen, wherein the neutralizing step comprises adjusting the macromolecular matrix to a physiological pH and physiological salt concentration.

In some embodiments of any one of each or any of the above- or below-mentioned embodiments, the neutralizing comprises adding a basic solution or buffered solution after the mixing step under aseptic conditions to adjust the pH. In some embodiments of any one of each or any of the above- or below-mentioned embodiments, adjusting the pH and physiological salt concentration causes precipitation of the collagen into fibrils or particles, wherein the collagen fibrils or particles are distributed homogeneously in the macromolecular matrix.

In some embodiments of any one of each or any of the above- or below-mentioned embodiments, the macromolecular matrix has enhanced extrudability through a needle, wherein the needle comprises a gauge size of 27G, 28G, 29G, 30G, 31G or 32G.

In a third aspect, a macromolecular matrix is provided, wherein the macromolecular matrix is prepared by any one of the embodiments described herein.

In some embodiments of any one of each or any of the above- or below-mentioned embodiments, the macromolecular matrix comprises crosslinked hyaluronic acid, wherein the collagen is physically mixed with crosslinked hyaluronic acid, and wherein the collagen is homogeneous throughout the macromolecular complex. In some embodiments of any one of each or any of the above- or below-mentioned embodiments, the collagen is evenly mixed throughout the macromolecular complex. In some embodiments of any one of each or any of the above- or below-mentioned embodiments, the macromolecular matrix comprises HA-Collagen conjugated gel, wherein the HA-Collagen conjugated gel also comprises physically mixed in collagen that is not crosslinked to the HA-Collagen conjugated gel. In some embodiments of any one of each or any of the above- or below-mentioned embodiments, the macromolecular matrix further comprises lidocaine. In some embodiments of any one of each or any of the above- or below-mentioned embodiments, the lidocaine is at a concentration in between a range of about 0.15% (w/w) to about 0.45% (w/w) in the matrix. In some embodiments of any one of each or any of the above- or below-mentioned embodiments, the lidocaine is at a concentration in between a range of about 0.27% (w/w) to about 0.33% (w/w) in the matrix. In some embodiments of any one of each or any of the above- or below-mentioned embodiments, the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w), about 0.39% (w/w), about 0.41% (w/w), about 0.43% (w/w), or about 0.45% (w/w) of the matrix, or any concentration in between a range defined by any two aforementioned values. In some embodiments of any one of each or any of the above- or below-mentioned embodiments, the lidocaine is at a concentration of about 0.3% (w/w) in the matrix.

In some embodiments of any one of each or any of the above- or below-mentioned embodiments, the matrix further comprises un-crosslinked HA. In some embodiments of any one of each or any of the above- or below-mentioned embodiments, the un-crosslinked HA comprises a concentration of up to 5% (w/w) within the matrix. In some embodiments of any one of each or any of the above- or below-mentioned embodiments, the un-crosslinked HA comprises a concentration of about 0% (w/w), about 1% (w/w), about 2% (w/w), about 3% (w/w), about 4% (w/w), about 5% (w/w) in the matrix or any concentration in between a range defined by any two aforementioned values. In some embodiments of any one of each or any of the above- or below-mentioned embodiments, the un-crosslinked HA comprises a concentration of about 1% (w/w) in the matrix. In some embodiments of any one of each or any of the above- or below-mentioned embodiments, the un-crosslinked HA comprises a concentration of about 2% (w/w) in the matrix. In some embodiments of any one of each or any of the above- or below-mentioned embodiments, the un-crosslinked HA comprises a concentration of about 5% (w/w) in the matrix. In some embodiments of any one of each or any of the above- or below-mentioned embodiments, the un-crosslinked HA, improves the extrudability of the matrix.

In a fourth aspect, a macromolecular matrix is provided, wherein the macromolecular matrix is made by a process described by any one of the embodiments of any one of each or any of the above- or below-mentioned embodiments. In some embodiments of any one of each or any of the above- or below-mentioned embodiments, the method further comprises concentrating the collagen prior to adding the collagen to the crosslinked HA.

In a fifth aspect, a macromolecular matrix is provided, wherein the macromolecular matrix comprises hyaluronic acid, wherein the hyaluronic acid is crosslinked; and collagen; wherein the collagen is physically mixed with the crosslinked hyaluronic acid. In some embodiments of any one of each or any of the above- or below-mentioned embodiments, the macromolecular matrix further comprises lidocaine. In some embodiments of any one of each or any of the above- or below-mentioned embodiments, the lidocaine is at a concentration in between a range of about 0.15% (w/w) to about 0.45% (w/w) in the matrix. In some embodiments of any one of each or any of the above- or below-mentioned embodiments, the lidocaine is at a concentration in between a range of about 0.27% (w/w) to about 0.33% (w/w) in the matrix. In some embodiments of any one of each or any of the above- or below-mentioned embodiments, the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w), about 0.39% (w/w), about 0.41% (w/w), about 0.43% (w/w), or about 0.45% (w/w) of the matrix, or any concentration in between a range defined by any two aforementioned values. In some embodiments of any one of each or any of the above- or below-mentioned embodiments, the lidocaine is at a concentration of 0.3% (w/w) in the matrix.

In some embodiments of any one of each or any of the above- or below-mentioned embodiments, the matrix further comprises un-crosslinked HA. In some embodiments of any one of each or any of the above- or below-mentioned embodiments, the un-crosslinked HA comprises a concentration of up to about 5% (w/w) within the matrix. In some embodiments of any one of each or any of the above- or below-mentioned embodiments, the un-crosslinked HA comprises a concentration of about 0% (w/w), about 1% (w/w), about 2% (w/w), about 3% (w/w), about 4% about (w/w), 5% (w/w) in the matrix, or any concentration in between a range defined by any two aforementioned values. In some embodiments of any one of each or any of the above- or below-mentioned embodiments, the un-crosslinked HA comprises a concentration of about 1% (w/w) in the matrix. In some embodiments of any one of each or any of the above- or below-mentioned embodiments, the un-crosslinked HA comprises a concentration of about 2% (w/w) in the matrix. In some embodiments of any one of each or any of the above- or below-mentioned embodiments, the un-crosslinked HA comprises a concentration of about 5% (w/w) in the matrix. In some embodiments of any one of each or any of the above- or below-mentioned embodiments, the un-crosslinked HA, improves the extrudability of the matrix.

In a sixth aspect, a macromolecular matrix is provided, wherein the macromolecular matrix comprises an HA-Collagen conjugated gel, wherein the HA-Collagen conjugated gel comprises hyaluronic acid crosslinked with collagen; and physically mixed in collagen, wherein the physically mixed in collagen is not crosslinked to the HA-Collagen conjugated gel, and wherein the physically mixed in collagen is mixed homogeneously within the HA-Collagen conjugated gel. In some embodiments of any one of each or any of the above- or below-mentioned embodiments, the macromolecular matrix further comprises lidocaine. In some embodiments of any one of each or any of the above- or below-mentioned embodiments, the lidocaine is at a concentration in between a range of about 0.15% (w/w) to about 0.45% (w/w) in the matrix. In some embodiments of any one of each or any of the above- or below-mentioned embodiments, the lidocaine is at a concentration in between a range of about 0.27% (w/w) to about 0.33% (w/w) in the matrix. In some embodiments of any one of each or any of the above- or below-mentioned embodiments, the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w)), about 0.39% (w/w), about 0.41% (w/w), about 0.43% (w/w), or about 0.45% (w/w)of the matrix, or any concentration in between a range defined by any two aforementioned values. In some embodiments of any one of each or any of the above- or below-mentioned embodiments, the lidocaine is at a concentration of about 0.3% (w/w) in the matrix.

In some embodiments of any one of each or any of the above- or below-mentioned embodiments, the matrix further comprises un-crosslinked HA. In some embodiments of any one of each or any of the above- or below-mentioned embodiments, the un-crosslinked HA comprises a concentration of up to about 5% (w/w) within the matrix. In some embodiments of any one of each or any of the above- or below-mentioned embodiments, the un-crosslinked HA comprises a concentration of about 0% (w/w), about 1% (w/w), about 2% (w/w), about 3% (w/w), about 4% (w/w), about 5% (w/w) in the matrix, or any concentration in between a range defined by any two aforementioned values. In some embodiments of any one of each or any of the above- or below-mentioned embodiments, the un-crosslinked HA comprises a concentration of about 1% (w/w) in the matrix. In some embodiments of any one of each or any of the above- or below-mentioned embodiments, the un-crosslinked HA comprises a concentration of about 2% (w/w) in the matrix. In some embodiments of any one of each or any of the above- or below-mentioned embodiments, the un-crosslinked HA comprises a concentration of about 5% (w/w) in the matrix. In some embodiments of any one of each or any of the above- or below-mentioned embodiments, the un-crosslinked HA, improves the extrudability of the matrix.

In some embodiments of any one of each or any of the above- or below-mentioned embodiments, the hyaluronic acid is crosslinked with a naturally occurring amine. In some embodiments of any one of each or any of the above- or below-mentioned embodiments, the naturally occurring amine is from lysine. In some embodiments of any one of each or any of the above- or below-mentioned embodiments, the collagen is from bovine collagen, marine collagen, human collagen or porcine collagen. In some embodiments of any one of each or any of the above-or below-mentioned embodiments, the collagen is recombinant human collagen.

In some embodiments of any one of each or any of the above- or below-mentioned embodiments, the macromolecular matrix comprises an elastic modulus (G′) of about 100 Pa, about 200 Pa, about 300 Pa, about 400 Pa, about 500 Pa, about 600 Pa, about 700 Pa, about 800 Pa, about 900 Pa, about 1000 Pa, about 1100 Pa, about 1200 Pa, about 1300 Pa, about 1400 Pa, about 1500 Pa, about 1600 Pa, about 1700 Pa, about 1800 Pa, about 1900 Pa, about 2000 Pa, about 2100 Pa, about 2200 Pa, about 2300 Pa, about 2400 Pa, about 2500 Pa, about 2600 Pa, about 2700 Pa, about 2800 Pa, about 2900 Pa, or about 3000 Pa or any value in between a range defined by any two aforementioned values.

In some embodiments of any one of each or any of the above- or below-mentioned embodiments, the macromolecular matrix comprises a compression force value of about 10 gmf, about 20 gmf, about 30 gmf, about 40 gmf, about 50 gmf, about 60 gmf, about 70 gmf, about 80 gmf, about 90 gmf, about 100 gmf, about 110 gmf, about 120 gmf, about 130 gmf, about 140 gmf, about 150 gmf, about 160 gmf, about 170 gmf, about 180 gmf, about 190 gmf, about 200 gmf, about 210 gmf, about 220 gmf, about 230 gmf, about 240 gmf, about 250 gmf, about 260 gmf, about 270 gmf, about 280 gmf, about 290 gmf, about 300 gmf, about 310 gmf, about 320 gmf, about 330 gmf, about 340 gmf, about 350 gmf, about 360 gmf, about 370 gmf, about 380 gmf, about 390 gmf, about 400 gmf, about 410 gmf, about 420 gmf, about 430 gmf, about 440 gmf, about 450 gmf, about 460 gmf, about 470 gmf, about 480 gmf, about 490 gmf, about 500 gmf, about 510 gmf, about 520 gmf, about 530 gmf, about 540 gmf, about 550 gmf, about 560 gmf, about 570 gmf, about 580 gmf, about 590 gmf or about 600 gmf or any compression force value in between a range defined by any two aforementioned values.

In some embodiments of any one of each or any of the above- or below-mentioned embodiments, the hyaluronic acid is at a concentration of about 2 mg/ml, about 4 mg/ml, about 6 mg/ml, about 8 mg/ml, about 10 mg/ml, about 12 mg/ml, about 14 mg/ml, about 16 mg/ml, about 18 mg/ml, about 20 mg/ml, about 22 mg/ml, about 24 mg/ml, about 26 mg/ml about 28 mg/ml, about 30 mg/ml, 32 mg/ml, about 34 mg/ml or about 36 mg/ml or any concentration in between a range defined by any two aforementioned values.

In some embodiments of any one of each or any of the above- or below-mentioned embodiments, the weight ratio of hyaluronic acid to collagen is at about 20:3, about 24:2.3, about 24: 10, about 24:12, about 24:4, about 24:6, about 28:6 or about 28:11.

In some embodiments of any one of each or any of the above- or below-mentioned embodiments, the hyaluronic acid comprises a mixture of hyaluronic acids, wherein the mixture comprises a 50:50 blend of HA comprising two different molecular weights.

In some embodiments of any one of each or any of the above- or below-mentioned embodiments, the collagen comprises Type I collagen and/or Type III collagen.

In some embodiments of any one of each or any of the above- or below-mentioned embodiments, the collagen is at a concentration of about 1 mg/ml, about 2 mg/ml, about 3 mg/ml, about 4 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml or about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml or any concentration in between a range defined by any two aforementioned values.

In some embodiments of any one of each or any of the above- or below-mentioned embodiments, the macromolecular matrix comprises an osmolality between 250 mOsm/kg — 350 mOsm/kg.

In some embodiments of any one of each or any of the above- or below-mentioned embodiments, the macromolecular matrix comprises an osmolality between about 250 mOsm/kg, about 275 mOsm/kg, about 300 mOsm/kg, about 325 mOsm/kg, or about 350 mOsm/kg or any osmolality in between a range defined by any two aforementioned values.

In some embodiments of any one of each or any of the above- or below-mentioned embodiments, the hyaluronic acid is linear.

In some embodiments of any one of each or any of the above- or below-mentioned embodiments, the macromolecular matrix comprises a viscous modulus (G″) of about 10 Pa, about 20 Pa, about 30 Pa, about 40 Pa, about 50 Pa, about 60 Pa, about 70 Pa, about 80 Pa, about 90 Pa, about 100 Pa, about 200 Pa, about 300 Pa, about 400 Pa, about 500 Pa, about 600 Pa, about 700 Pa, about 800 Pa, about 900 Pa or about 1000 Pa or any viscous modulus (G″) in between a range defined by any two aforementioned values.

In some embodiments of any one of each or any of the above- or below-mentioned embodiments, the macromolecular matrix comprises a tan delta (G″/G′) of about 0.01, about 0.02, about 0.03, about 0.04, about 0.05, about 0.06, about 0.07, about 0.08, about 0.09, about 0.1, about 0.12, about 0.14, about 0.16, about 0.18, about 0.20, about 0.22, about 0.24, about 0.26, about 0.28, about 0.30, about 0.32, about 0.34, about 0.36, about 0.38, about 0.40, about 0.42, about 0.44, about 0.46, about 0.48, about 0.50 or any tan delta (G″/G′) in between a range defined by any two aforementioned values.

In some embodiments of any one of each or any of the above- or below-mentioned embodiments, the macromolecular matrix comprises a pH of about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0 or any pH in a range defined by any two aforementioned values.

In a seventh aspect, a method of improving an aesthetic quality of an anatomic feature of a human being is provided. The method comprises injecting a composition into a tissue of the human being to thereby improve the aesthetic quality of the anatomic feature; wherein the composition comprises the macromolecular matrix prepared by the method of any one of the embodiments described herein or the macromolecular matrix of any one of the embodiments described herein.

In an eighth aspect, a method of improving an aesthetic quality of an anatomic feature of a human being is provided, the method comprising: injecting a composition into a tissue of the human being to thereby improve the aesthetic quality of the anatomic feature; wherein the composition comprises a macromolecular matrix comprising: crosslinked hyaluronic acid; lysine; and collagen; wherein the collagen is physically mixed into the crosslinked hyaluronic acid.

In some embodiments of any one of each or any of the above- or below-mentioned embodiments, the collagen comprises collagen type I and/or collagen type III.

In some embodiments of any one of each or any of the above- or below-mentioned embodiments, the method does not cause a bluish discoloration at a site of injection.

In an ninth aspect, a method of improving the appearance of a human being by injecting a composition into the tissue of a human being whereby the composition promotes cell infiltration and collagen deposition into the composition from the surrounding tissue into which it was injected, is provided. The method comprises injecting a composition into a tissue of the human being to thereby improve the aesthetic quality of the anatomic feature; wherein the composition comprises a crosslinked macromolecular matrix comprising: hyaluronic acid, lysine, and collagen; wherein the hyaluronic acid comprises crosslinked hyaluronic acid that is physically mixed with the collagen; and wherein the tissue injected by the composition is shown to integrate with the composition. In some embodiments of any one of each or any of the above- or below-mentioned embodiments, the macromolecular matrix further comprises lidocaine. In some embodiments of any one of each or any of the above- or below-mentioned embodiments, the lidocaine is at a concentration in between a range of about 0.15% (w/w) to about 0.45% (w/w) in the matrix. In some embodiments of any one of each or any of the above- or below-mentioned embodiments, the lidocaine is at a concentration in between a range of about 0.27% (w/w) to about 0.33% (w/w) in the matrix. In some embodiments of any one of each or any of the above- or below-mentioned embodiments, the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w), about 0.39% (w/w), about 0.41% (w/w), about 0.43% (w/w), or about 0.45% (w/w) of the matrix, or any concentration in between a range defined by any two aforementioned values. In some embodiments of any one of each or any of the above- or below-mentioned embodiments, the lidocaine is at a concentration of 0.3% (w/w) in the matrix.

In some embodiments of any one of each or any of the above- or below-mentioned embodiments, the matrix further comprises un-crosslinked HA. In some embodiments of any one of each or any of the above- or below-mentioned embodiments, the un-crosslinked HA comprises a concentration of up to about 5% (w/w) within the matrix. In some embodiments of any one of each or any of the above- or below-mentioned embodiments, the un-crosslinked HA comprises a concentration of 0% (w/w), about 1% (w/w), about 2% (w/w), about 3% (w/w), about 4% (w/w), or about 5% (w/w) in the matrix or any concentration in between a range defined by any two aforementioned values. In some embodiments of any one of each or any of the above- or below-mentioned embodiments, the un-crosslinked HA comprises a concentration of about 1% (w/w) in the matrix. In some embodiments of any one of each or any of the above- or below-mentioned embodiments, the un-crosslinked HA comprises a concentration of about 2% (w/w) in the matrix. In some embodiments of any one of each or any of the above- or below-mentioned embodiments, the un-crosslinked HA comprises a concentration of about 5% (w/w) in the matrix. In some embodiments of any one of each or any of the above- or below-mentioned embodiments, the un-crosslinked HA, improves the extrudability of the matrix.

In some embodiments of any one of each or any of the above- or below-mentioned embodiments, the hyaluronic acid component has an average molecular weight of about 10,000 Daltons, about 20,000 Daltons, about 30,000 Daltons, 40,000 Daltons, about 50,000 Daltons, about 60,000 Daltons, about 70,000 Daltons, about 80,000 Daltons, about100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,100,000 Daltons, about 1,200,000 Daltons, about 1,300,000 Daltons, about 1,400,000 Daltons, about 1,500,000 Daltons, about 1,600,000 Daltons, about 1,700,000 Daltons, about 1,800,000 Daltons, about 1,900,000 Daltons, about 2,000,000 Daltons, about 2,100,000 Daltons, about 2,200,000 Daltons, about 2,300,000 Daltons, about 2,400,000 Daltons, about 2,500,000 Daltons, about 2,600,000 Daltons, about 2,700,000 Daltons, about 2,800,000 Daltons, about 2,900,000 Daltons, about 3,000,000 Daltons, about 3,100,000 Daltons, about 3,200,000 Daltons, about 3,300,000 Daltons, about 3,400,000 Daltons, about 3,500,000 Daltons, about 3,600,000 Daltons, about 3,700,000 Daltons, about 3,800,000 Daltons, about 3,900,000 Daltons, about 4,000,000 Daltons, about 4,100,000 Daltons, about 4,200,000 Daltons, about 4,300,000 Daltons, about 4,400,000 Daltons, about 4,500,000 Daltons, about 4,600,000 Daltons, about 4,700,000 Daltons, about 4,800,000 Daltons, about 4,900,000 Daltons, about 5,000,000 Daltons, about 5,100,000 Daltons, about 5,200,000 Daltons, about 5,300,000 Daltons, about 5,400,000 Daltons, about 5,500,000 Daltons, about 5,600,000 Daltons, about 5,700,000 Daltons, about 5,800,000 Daltons, about 5,900,000 Daltons, about 6,000,000 Daltons, about 6,100,000 Daltons, about 6,200,000 Daltons, about 6,300,000 Daltons, about 6,400,000 Daltons, about 6,500,000 Daltons, about 6,600,000 Daltons, about 6,700,000 Daltons, about 6,800,000 Daltons, about 6,900,000 Daltons, about 7,000,000 Daltons, about 7,100,000 Daltons, about 7,200,000 Daltons, about 7,300,000 Daltons, about 7,400,000 Daltons, about 7,500,000 Daltons, about 7,600,000 Daltons, about 7,700,000 Daltons, about 7,800,000 Daltons, about 7,900,000 Daltons, about 8,000,000 Daltons, about 8,100,000 Daltons, about 8,200,000 Daltons, about 8,300,000 Daltons, about 8,400,000 Daltons, about 8,500,000 Daltons, about 8,600,000 Daltons, about 8,700,000 Daltons, about 8,800,000 Daltons, about 8,900,000 Daltons, about 9,000,000 Daltons, about 9,100,000 Daltons, about 9,200,000 Daltons, about 9,300,000 Daltons, about 9,400,000 Daltons, about 9,500,000 Daltons, about 9,600,000 Daltons, about 9,700,000 Daltons, about 9,800,000 Daltons, about 9,900,000 Daltons or about 10,000,000 Daltons or any weight in between a range defined by any two aforementioned values. In some embodiments of any one of each or any of the above- or below-mentioned embodiments, the hyaluronic acid of the crosslinked hyaluronic acid comprises a mixture of hyaluronic acid components comprising at least two different molecular weights.

In some embodiments of any one of each or any of the above- or below-mentioned embodiments, the collagen comprises collagen type I and/or collagen type III.

In some embodiments of any one of each or any of the above- or below-mentioned embodiments, the tissue injected by the composition is shown to integrate with the composition with collagen deposition within the composition by infiltrating cells from surrounding tissue.

In some embodiments of any one of each or any of the above- or below-mentioned embodiments, the tissue injected by the composition is shown to integrate with the composition with collagen deposition within the injected composition after injecting the composition.

### BRIEF DESCRIPTION OF THE DRAWINGS

FIG. 1 shows several examples of different compositions of hydrogels that have varying amounts of collagen and hyaluronic acid. The opacity of the hydrogels increases with increasing collagen concentration for gels containing about 6.5 mg/ml HA and gels containing about 12.7 mg/ml hyaluronic acid.

FIG. 2 shows G′, G″, and tan δ of HA-Collagen hydrogels at 25° C. with 5 Hz frequency and 0.8% strain between two parallel plates 25 mm in diameter and 1 mm gap length.

FIG. 3 shows swelling of the hydrogel formulations.

FIG. 4 shows change in compression force from a 2.5 to 0.9 mm gap for HA-Collagen formulations.

FIG. 5 shows average extrusion force of HA-Collagen hydrogels through a 27G-½ needle using a 1 mL BD tuberculin syringe.

FIG. 6 shows mean height over time of BDDE crosslinked and naturally occurring amine (NOA) crosslinked HA hydrogel samples implanted in a rat model showing the lift of NOA crosslinked formulations with increasing HA concentration.

FIG. 7 shows mean height over time of BDDE crosslinked and NOA crosslinked HA-Collagen hydrogel samples implanted in a rat model showing the effect of collagen mixing (<3 mg/mL) on lift capacity.

FIG. 8 shows mean height over time of BDDE crosslinked and NOA crosslinked HA-Collagen hydrogel samples implanted in a rat model showing the effect of collagen mixing (up to 11 mg/mL) on lift capacity.

FIG. 9 shows cell viability of human dermal fibroblasts cultured on BDDE crosslinked and NOA crosslinked HA-Collagen hydrogels. * p<0.05, ANOVA with Tukey post-hoc.

FIG. 10 shows cell morphology analysis of human dermal fibroblasts cultured on NOA hydrogel or NOA crosslinked HA-Collagen hydrogel (24 mg/mL HA, 6 mg/mL collagen). * p<0.05, Students t-test.

FIG. 11 shows immunohistochemistry for rat Collagen Type I showing increased collagen deposition in 20 mg/mL HA hydrogel boluses containing 3 mg/mL collagen.

FIG. 12 shows immunohistochemistry for rat Collagen Type I showing encapsulation of the center of the hydrogel bolus containing 28 mg/mL HA. The addition of up to 11 mg/mL collagen results in moderate improvement in the collagen deposition along the periphery of the hydrogel bolus.

FIG. 13 shows immunohistochemistry for rat Collagen Type I showing increasing collagen deposition within the 24 mg/mL HA hydrogel boluses with increasing collagen concentration (2.3 mg/mL to 6 mg/mL). The formulation containing 24 mg/mL HA and 6 mg/mL collagen (24NOA6CN) exhibits the most robust tissue integration.

FIG. 14 shows immunohistochemistry for rat Collagen Type I showing the collagen deposition/tissue integration within BDDE crosslinked HA and NOA crosslinked HA-Collagen hydrogels from 4 to 12 weeks after subcutaneous implantation in rats. The formulation containing 24 mg/mL HA and 6 mg/mL collagen (24NOA6CN) exhibits more robust tissue integration at 4 and 12 weeks than the BDDE crosslinked HA only gel. The tissue integration appears to improve from 4 to 12 weeks in the 24NOA6CN formulation.

FIG. 15 shows semi-quantitative histopathology scoring of the tissue integration into BDDE crosslinked HA only hydrogel and 24NOA6CN hydrogel. Scoring system: Best integration=10, Worst Integration=−5. (A) Colloidal iron staining showing enhanced tissue integration into the 24NOA6CN hydrogel bolus and the associated histopathology score. (B) Bar graphs showing the histopathological scoring of HA only BDDE crosslinked hydrogel and 24NOA6CN after 4 and 12 weeks subcutaneous implantation in rats. The 24NOA6CN gel exhibits the highest tissue integration score after 12 weeks.

### DETAILED DESCRIPTION

Factors such as sun exposure, genetics, disease, injury and lifestyle can break down our underlying tissues and lead to undesirable changes in our skin that result in wrinkles, folds and sagging of tissue leading to the visible effects of aging. These changes can be attributed in large part to loss of fat tissue and extracellular matrix proteins such as collagen and elastin. Loss of these components can contribute to thinning of the layers of the skin, which together with changes in bone and muscle structure, can lead to loss in volume and formation of lines. To return skin back to its previous youthful appearance current approaches aim at restoring volume and increasing thickness of the skin.

Dermal fillers are one of many treatments available for erasing lines and restoring volume. Following an injection, the dermal filler serves to take the place of lost collagen and elastin thus making the skin thicker, lifting the tissue and ultimately removing the lines. The effects of the dermal fillers are temporary and are gradually lost over time. As a result, subsequent injections are required to maintain a youthful appearance.

Hyaluronic acid (HA) dermal fillers are very effective at removing lines and are dominating the market for several reasons. The dermal filler may be modified to affect the duration of the filler in the tissue. Without being limiting, modifications such as altering the crosslinking of the dermal filler may affect the duration of the filler in tissues, for example.

Aside from its long-lasting benefits, HA dermal fillers are also reversible. Reversibility is a key benefit especially if the filler is incorrectly placed or when an adverse event (AE), such as an occluded blood vessel, occurs. For example, in the case of an AE, the dermal filler can be quickly degraded by injecting hyaluronidase in the affected area. Hyaluronidase is an enzyme that breaks down HA and converts the gel filler into a liquid solution which can diffuse away from the affected site. These benefits have made HA dermal fillers one of the most popular fillers on the market today.

Despite the benefits of HA dermal fillers, their performance can be improved. For example, HA fillers do not replenish and/or significantly stimulate the synthesis of extra-cellular matrix (ECM) proteins associated with youth, such as collagen. Additionally, if injected superficially, HA dermal fillers may lead to an AE that manifests itself as a bluish discoloration at the surface of the skin known as the Tyndall effect (Cohen et al. Understanding, avoiding, and managing dermal filler complications. Dermatol Surg, 2008. 34 Suppl 1: p. S92-9; incorporated by reference herein). This is a result of the transparent particles in the dermal filler scattering light in a way that leads to a bluish tint. The injected HA product as well as the anatomic region of injection can both affect the probability of exhibiting the Tyndall effect (Bailey et al. Etiology, prevention, and treatment of dermal filler complications. Aesthet Surg J, 2011. 31(1): p. 110-21; incorporated by reference herein). Swelling can also be a concern with HA fillers, since HA is extremely hydrophilic and strongly attracts water. Hence, as the concentration of HA in a hydrogel increases or the degree of crosslinking decreases, the absorption of water also increases. For example, some HA fillers can absorb more than 6 times their starting weight when exposed to saline water. The absorption of water can lead to swelling, especially when the filler is injected superficially or injected in an area with thin skin. Additionally, the available fillers in the market comprising only HA, were not shown to allow tissue integration. Regardless of these concerns, HA dermal fillers are still the most popular fillers on the market because of their performance.

Collagen dermal fillers were introduced to the market in 1981. Collagen is an ideal material for this application as it is a natural component of the skin giving it strength, fullness, and texture. When injected, these fillers add back collagen to the skin thus replenishing an ECM protein associated with youth. Additionally, the gels do not lead to the Tyndall effect. For example, collagen dermal fillers like Zyderm® 1 and Cosmoderm® 1 were recommended for injection into the papillary dermis (Gold et al. Use of hyaluronic acid fillers for the treatment of the aging face. Clinical Interventions in Aging, 2007. 2(3): p. 369-376; incorporated by reference herein). Even though they were injected superficially, the opaque appearance of the materials prevented the observance of a Tyndall effect. Hence, collagen fillers offer some benefits over HA based fillers.

Despite these benefits, collagen fillers have some drawbacks that make HA fillers preferable. For example, collagen fillers cannot be reversed using injectable, commercially available enzyme solutions, as for HA fillers. Collagen dermal fillers are offered with high collagen concentrations (35-65 mg/mL collagen) to achieve the desired result and this high collagen concentration could be difficult to remove in event of an adverse reaction. Additionally, collagen dermal fillers have demonstrated limited duration, averaging between 3 and 6 months of correction. Crosslinking of the collagen can lead to a more robust hydrogel which can be placed deeper in the skin and can lead to improved duration. However, the crosslinked collagen gel is less similar than un-crosslinked collagen fillers to the collagen present in the body and is therefore, less natural.

In the following embodiments, the preparation and characterization of a hybrid material made of HA fillers containing physically mixed collagen are described. The material takes advantage of the benefits offered by both collagen and HA fillers while eliminating the concerns each material presents alone.

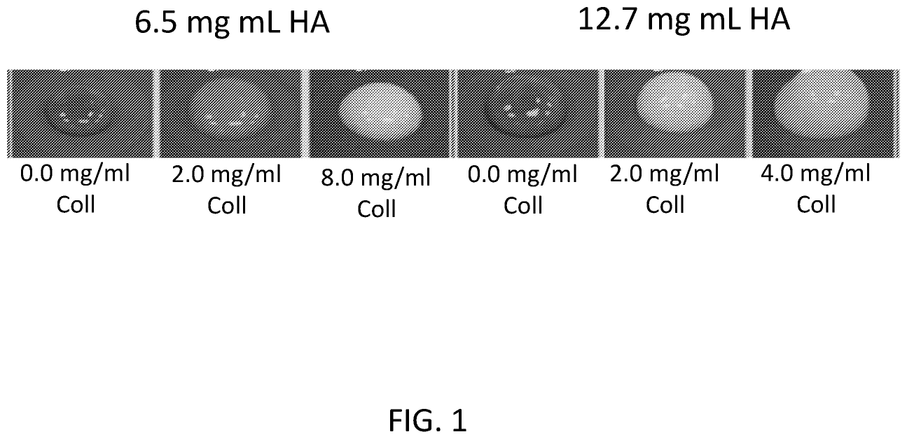

Due to the hybrid nature of the material, the benefits of HA dermal fillers are retained while the inclusion of collagen positively addresses the concerns. For example, since the addition of collagen to the HA gel results in an opaque formulation (see Example 2 and FIG. 1) which can address the concern of the Tyndall effect. Additionally, inclusion of collagen allows for high gel stiffness without altering the swelling properties. With HA fillers alone, G′ (i.e. stiffness) can be increased by increasing the HA concentration or increasing the crosslinking density of the hydrogel network. However, increased HA concentration results in increased swelling while more highly crosslinked gels demonstrate lower swelling. Therefore, physically mixed HA collagen hydrogels are unique in that G′/stiffness increases with collagen concentration, without affecting swelling properties (Examples 3 and 4). Lastly, because collagen is associated with youthful skin, injection of compositions of HA filler mixed with collagen can replenish ECM proteins associated with youthful skin. HA fillers mixed with collagen also serve as scaffolds for tissue ingrowth and new collagen deposition after injection. This enhanced tissue integration may translate into more natural, long lasting effects. Thus, when collagen was incorporated into the HA dermal filler, it surprisingly improves the physical properties, as well as the biological performance, of the final product.

Likewise, the shortcomings of collagen dermal fillers can be addressed by inclusion of HA dermal fillers in the formulation. For example, the short duration of collagen fillers can be improved by using a longer-lasting HA dermal filler mixed with collagen. Additionally, significantly less collagen is needed for formulations of HA fillers mixed with collagen to achieve the desired biological effect and therefore, reversibility of the formulation is enhanced over collagen-only dermal fillers since the HA portion of the composition can be degraded using injected hyaluronidase. With the HA-collagen gels, the filling effect is due to the HA dermal filler. If an AE is encountered, hyaluronidase can be used to degrade HA, thus turning the material to liquid and ensuring the safety of the patient. Therefore, mixed HA dermal filler with collagen formulations provide substantial benefits over using HA or collagen alone.

As described in the embodiments herein, the mixed HA and collagen compositions led to hydrogels with surprising beneficial properties. It was found that different combined concentration of HA and collagen led to hydrogels that allowed lift as well as tissue integration. In some embodiments, the compositions comprising 20 mg/ml HA or 24 mg/ml HA led to good lift in the tissues. However, some compositions in which the concentrations were increased to at least 28 mg/ml led to improved lift but did not improve tissue integration into the injection site. In some embodiments, the compositions comprised 24 mg/ml HA and 6 mg/ml collagen. In some embodiments, the compositions comprised 20 mg/ml HA and 10 mg/ml collagen. In some embodiments, the composition led to tissue lift as well as enhanced tissue integration into the composition injection site. In some embodiments, the composition had good lift and higher tissue integration compared to an HA only gel.

Ease of injectability is also a key property of dermal fillers and can be quantified using an extrusion force (EF) measurement. Essentially, EF is the force required to be applied to the plunger in order to extrude the gel through a specific gauge needle. An easily-injected dermal filler will be characterized by an extrusion force which is lower in magnitude and an extrusion profile which is smooth and uniform over the length of the syringe. A force of<40 N is considered acceptable and allows for ease of injection, as well as control and precision, during the injection. The average extrusion force of the HA collagen formulations is substantially less than 40 N (see example 6). In some embodiments herein, the macromolecular matrix has an extrusion force less than 40 N. Additionally, the extrusion force curves appear smooth suggesting that the materials are homogeneous. Therefore, the HA collagen dermal fillers are easily injected through a fine gauge needle. In some embodiments of any one of each or any of the above- or below-mentioned embodiments, the macromolecular complex further comprises un-crosslinked HA. In some embodiments of any one of each or any of the above- or below-mentioned embodiments, the un-crosslinked HA is at a concentration in the macromolecular complex of 1%, 2% or 5%. In some embodiments of any one of each or any of the above- or below-mentioned embodiments, the un-crosslinked HA leads to ease in extrudability and decreases the extrusion force of the composition.

These embodiments herein specifically describes HA dermal fillers physically mixed with collagen. Alternatively, collagen can be chemically crosslinked with HA to form a different type of HA collagen gel. However, when compared to chemically crosslinked gels, physically mixed gels contain collagen that more closely resembles the collagen found in native tissue.

In some embodiments of any one of each or any of the above- or below-mentioned embodiments, the compositions lead to improvement in skin quality, such as the hydration of the skin, improved elasticity and decreased fine lines of the skin.

Unless defined otherwise, all technical and scientific terms used herein have the same meaning as commonly understood by one of ordinary skill in the art to which the inventions pertain.

As used herein, “a” or “an” may mean one or more than one. “About” as used herein when referring to a measurable value is meant to encompass variations of +20% or +10%, more preferably +5%, even more preferably +1%, and still more preferably +0. 1% from the specified value.

As used herein, except where the context requires otherwise, the term ‘comprise’ and variations of the term, such as “comprising,” “comprises” and “comprised,” are not intended to exclude further additives, components, integers or steps.

The term “hyaluronic acid” or “HA” is a non-sulfated glycosaminoglycan that is distributed widely throughout the human body in connective, epithelial, and neural tissues. Without being limiting, this sodium hyaluronate may also be used. Hyaluronan is abundant in the different layers of the skin, where it has multiple functions such as, e.g., to ensure good hydration, to assist in the organization of the extracellular matrix, to act as a filler material; and to participate in tissue repair mechanisms. However, with age, the quantity of hyaluronan, collagen, elastin, and other matrix polymers present in the skin decreases. For example, repeated exposed to ultra violet light, e.g., from the sun, causes dermal cells to both decrease their production of hyaluronan as well as increase the rate of its degradation. This hyaluronan loss results in various skin conditions such as, e.g., imperfects, defects, diseases and/or disorders, and the like. For instance, there is a strong correlation between the water content in the skin and levels of hyaluronan in the dermal tissue. As skin ages, the amount and quality of hyaluronan in the skin is reduced. These changes lead to drying and wrinkling of the skin.

HA may include hyaluronic acid and any of its hyaluronate salts, including, for example, sodium hyaluronate (the sodium salt), potassium hyaluronate, magnesium hyaluronate, and calcium hyaluronate. Hyaluronic acid from a variety of sources may be used herein. For example, hyaluronic acid may be extracted from animal tissues, harvested as a product of bacterial fermentation, or produced in commercial quantities by bioprocess technology. In the embodiments described herein, the crosslinked hyaluronic acid comprises a molecular weight of about 10,000 Daltons to about 10,000,000 Daltons.

A “macromolecular matrix” refers to a matrix formed by crosslinked HA that is physically mixed with collagen. In some embodiments, a macromolecular matrix further comprises collagen crosslinked to the HA.

“Collagen” as described herein is a structural protein that is found in various connective tissues in the body. Any type of collagen may be used in the methods and compositions described herein. In some embodiments, collagen type I, collagen type II, collagen type III, collagen type IV, collagen type VI, or a combination thereof, may be used. A collagen may be derived from cell culture, animal tissue, or recombinant means, and may be derived from human, porcine, recombinant or bovine sources, for example. Some embodiments comprise collagen derived from human fibroblast culture. Some embodiments comprise collagen that has been denatured to gelatin. In some embodiments of the macromolecular complex, the macromolecular complex comprises Type I and/or Type III collagen.

One of skill in the art would understand the considerations for different sources for collagen. Histopathologically, bovine collagen fibers may be thicker than human collagen, have a homogeneous appearance nearly devoid of spaces between them, with fewer fibroblasts, and fail to refract polarized light. Skin tests may be required before the injection of bovine or other animal collagen products. Rare hypersensitive reactions, including foreign body granulomas and palisading granulomas to bovine collagen have been reported. Rare systemic complications include flulike symptoms, paresthesias or difficulty breathing, and severe anaphylactic shock have been reported after injections of bovine collagen. As such, skin testing before injection may be required to identify patients at risk for allergic reactions and its short duration of effect. One of skill in the art would consider the types of collagen to be used in the embodiments herein and would appreciate the differences between sources of collagen.

In some embodiments of any one of each or any of the above- or below-mentioned embodiments, the compositions further comprises particles of HA or particles or fibrils of collagen.

As described herein, “localized delivery” refers to the administration of the composition into or near the tissue in need of the therapeutic composition. In some embodiments of any one of each or any of the above- or below-mentioned embodiments, the macromolecular matrix is locally delivered into an area on the body of the patient having a volume deficit.

Methods of treatment

In some embodiments of any one of each or any of the above- or below-mentioned embodiments, the composition is administered as an injection into facial soft tissue to increase size, shape, and/or contour of a facial feature like increased size, shape, and/or contour of lip, cheek or eye region; altered size, shape, and/or contour of facial feature like altered size, shape, and/or contour of lip, cheek or eye region shape; reduction or elimination of a wrinkle, fold or line in the skin; resistance to a wrinkle, fold or line in the skin; rehydration of the skin; increased elasticity to the skin; reduction or elimination of skin roughness; increased and/or improved skin tautness. Without being limiting, these areas may include the eye area, cheek area and/or neck area, for example.

One of skill in the art would understand that prior to administration, aseptic techniques may be used, such as proper skin sterilization with 2-4% chlorhexidine or 70% isopropyl alcohol solution and avoiding contamination of the treatment area after cleansing the patient's skin. An injection approach should be used that reduces the number of skin piercings and uses the smallest gauge needle possible for injections. It is also important to avoid injecting into inflamed or infected skin.

In some embodiments of any one of each or any of the above- or below-mentioned embodiments, the compositions described herein, provides immediate lift and volume upon injection while delivering and sequestering collagen. In the embodiments described herein, the methods provide a natural looking effect on the patient receiving the injection.

In some embodiments of any one of each or any of the above- or below-mentioned embodiments, the collagen is not chemically bound to the crosslinked HA network.

In some embodiments of any one of each or any of the above- or below-mentioned embodiments, the HA component is linear HA. In some embodiments of any one of each or any of the above- or below-mentioned embodiments, the HA is crosslinked with a naturally occurring amine using EDC and NHS chemistry or BDDE crosslinked HA. In some embodiments of any one of each or any of the above- or below-mentioned embodiments, the naturally occurring amine is lysine.

In some embodiments of any one of each or any of the above- or below-mentioned embodiments, the composition stimulates the synthesis of essential building blocks of skin, such as ECM proteins. Without being limiting proteins such as collagen production may be stimulated from the tissue into which the composition was injected.

In some embodiments of any one of each or any of the above- or below-mentioned embodiments, the methods of treatment are reversible upon hyaluronidase treatment. A hyaluronidase treatment may be provided during an adverse reaction or if the results are unfavorable.

In some embodiments of any one of each or any of the above- or below-mentioned embodiments, the composition may be used to superficially eliminate fine lines and deep (subcutaneous and/or supraperiosteal) for cheek augmentation to correct age-related volume deficit in the mid-face.

In some embodiments of any one of each or any of the above- or below-mentioned embodiments, the methods of treatment do not lead to a Tyndall effect in the sites of injection.

One of skill in the art would appreciate the different methods in which the macromolecular matrix may be administered to a patient in need. In some embodiments of any one of each or any of the above- or below-mentioned embodiments, the macromolecular matrix is administered with a needle or a cannula.

The collagen used in the embodiments herein may be provided from any source. Without being limiting, porcine, bovine, recombinant and human collagen may be used, for example. In some embodiments, the compositions described do not require allergy testing.

As described herein, the HA collagen dermal filler may provide immediate lift and volume upon injection while at the same time delivering and trapping collagen. In some embodiments of any one of each or any of the above- or below-mentioned embodiments, the composition may stimulate the synthesis of essential building blocks which leads to youthful looking and healthy skin. In some embodiments of any one of each or any of the above- or below-mentioned embodiments, the methods are reversible upon hyaluronidase treatment. In some embodiments of any one of each or any of the above- or below-mentioned embodiments, the compositions allows for increase in modulus (stiffness), while not significantly affecting the swelling properties of the filler.

In some embodiments of any one of each or any of the above- or below-mentioned embodiments, the compositions may superficially eliminate fine lines and deep (subcutaneous and/or supraperiosteal) lines for cheek augmentation to correct age related volume deficit in the mid-face.

In some embodiments of any one of each or any of the above- or below-mentioned embodiments, the compositions generate little to no granuloma throughout the lifetime of the filler. Therefore the filler does not lead to a “cobblestone” look upon the skin or within the area of the injection.

In some embodiments of any one of each or any of the above- or below-mentioned embodiments, the composition provides benefits such as hydration and rejuvenation of the skin.

Methods of making the Hydrogel

In some embodiments of any one of each or any of the above- or below-mentioned embodiments, the method of making the hydrogel comprises providing: HA, collagen, water, and PBS, and mixing at an acidic pH. The HA used in the method may be crosslinked. The collagen may be provided by any source. Without being limiting, the collagen may be porcine collagen, human collagen or a recombinant form of collagen.

There are different methods of mixing the collagen into the hyaluronic acid or into the mixture above. One of skill in the art would appreciate the variety of methods for making the hydrogel. Without being limiting, the collagen may be mixed into the crosslinked hyaluronic acid with a Thinky mixer, acoustic mixer, reactor, syringe-syringe mixing, stainless steel cartridges, for example.

In some embodiments of any one of the above- or below-embodiments, the mixture comprises a HA concentration of about 5 mg/ml, about 10 mg/ml, about 15 mg/ml, about 20 mg/ml, about 24 mg/ml, about 28 mg/ml, or any concentration within a range in between any two aforementioned values. In some embodiments of any one of the above- or below-embodiments, the mixture comprises a HA concentration of about 5 mg/ml. In some embodiments of any one of the above- or below-embodiments, the mixture comprises a HA concentration of about 25 mg/ml.

In some embodiments, wherein the HA concentration comprises about 20 mg/ml, the composition provides lift and tissue integration.

In some embodiments of any one of the above- or below- embodiments, the composition comprises 24 mg/ml HA and about 6 mg/ml collagen.

In some embodiments of any one of the above- or below- embodiments, the composition comprises 20 mg/ml HA and about 10 mg/ml collagen

In some embodiments of any one of the above- or below- embodiments, the method further comprises sterilizing the composition. There are many methods of sterilizing a hydrogel and these methods may be appreciated by one of skill in the art. Without being limiting, the composition may be sterilized by heat (dry heat, steam heat) radiation (non-ionizing, ionizing (particulate (e.g. gamma rays), electromagnetic (e.g. e-beam), filtration) or terminal sterilization (sterilization of a product in its final container, such as a syringe, for example), for example. In some embodiments of any one of the above- or below- embodiments, the composition may be sterilized by heat (dry heat, steam heat) radiation (non-ionizing, ionizing (particulate (e.g. gamma rays), electromagnetic (e.g. e-beam), filtration or terminal sterilization (sterilization of a product in its final container, such as a syringe).

Steam heat may be used to sterilize the product by exposing the composition to saturated steam under pressure, for example. For example, this may be performed for 15 minutes at 121 to 124° C. In some alternatives, different amounts of time and higher temperatures may be used, for example.

Dry heat may be used and may require a higher temperature and longer exposure time (Galante et al. 2017). In some embodiments of any of the above- or below-embodiments, a method for preparing a macromolecular matrix is provided wherein a crosslinked HA hydrogel is provided and collagen is provided, wherein the collagen is in a soluble state in a solution, and wherein the collagen is physically mixed into the crosslinked HA hydrogel.

In some embodiments of any of the above- or below- embodiments, the collagen is provided in a solution, wherein the solution is acidic. In some embodiments of any of the above- or below- embodiments, the collagen is provided in a solution state. In some embodiments of any of the above- or below- embodiments, the collagen is in a soluble state. In some embodiments of any of the above- or below- embodiments, the method of making the macromolecular matrix further comprises neutralizing the mixture comprising the crosslinked hyaluronic acid and mixed in collagen. In some embodiments of any of the above- or below-embodiments, collagen fibrils or collagen precipitates are formed after mixing. However, the collagen fibrils or collagen precipitates will be mixed into the HA hydrogel homogeneously. In some embodiments of any of the above- or below- embodiments, the method further comprises adjusting the osmolarity of the hydrogel after mixing the soluble collagen with the crosslinked HA. In some embodiments, the adjusting step may cause the collagen to form precipitates or fibrils, however the collagen precipitates or fibrils are mixed homogenously about the crosslinked hyaluronic acid.

In the embodiments described herein, it is important that the HA is crosslinked and that the collagen is added as a solution. The mixture should be mixed such that the mixture is well mixed and homogeneous. The mixture is then neutralized and the osmolarity is adjusted such that the macromolecular matrix is at the correct pH/osmolarity. In essence, it is important that composition be uniform and homogenous so that if may perform the function as a filler with properties such as lift capabilities and allowing tissue integration.

In some embodiments, collagen (in an acidic solution) is neutralized prior to mixing into the crosslinked hyaluronic acid. In some embodiments, the neutralizing to a pH of about 7, leads to different collagen structures, for example, fibrillation of the collagen. In some embodiments, these fibrils are mixed in homogeneously into the crosslinked hyaluronic acid.

In some embodiments, a crosslinked hyaluronic acid and collagen are mixed together at an acidic pH. After mixing the pH of the solution may be raised to a pH between 7.0 and 7.5 by adding a base and mixing a second time. In some embodiments, an increase of the pH may initiate self-assembly of a collagen network. In some embodiments, the mixture may be autoclaved after mixing.

Unless defined otherwise, all technical and scientific terms used herein have the same meaning as commonly understood by one of ordinary skill in the art to which the inventions pertain.

As used herein, “a” or “an” may mean one or more than one. “About” as used herein when referring to a measurable value is meant to encompass variations of +20% or +10%, more preferably +5%, even more preferably +1%, and still more preferably +0. 1% from the specified value.

As used herein, except where the context requires otherwise, the term ‘comprise’ and variations of the term, such as “comprising,” “comprises” and “comprised,” are not intended to exclude further additives, components, integers or steps.

The term “hyaluronic acid” or “HA” is a non-sulfated glycosaminoglycan that is distributed widely throughout the human body in connective, epithelial, and neural tissues. Hyaluronan is abundant in the different layers of the skin, where it has multiple functions such as, e.g., to ensure good hydration, to assist in the organization of the extracellular matrix, to act as a filler material; and to participate in tissue repair mechanisms. However, with age, the quantity of hyaluronan, collagen, elastin, and other matrix polymers present in the skin decreases. For example, repeated exposed to ultra violet light, e.g., from the sun, causes dermal cells to both decrease their production of hyaluronan as well as increase the rate of its degradation. This hyaluronan loss results in various skin conditions such as, e.g., imperfects, defects, diseases and/or disorders, and the like. For instance, there is a strong correlation between the water content in the skin and levels of hyaluronan in the dermal tissue. As skin ages, the amount and quality of hyaluronan in the skin is reduced. These changes lead to drying and wrinkling of the skin.

HA may include hyaluronic acid and any of its hyaluronate salts, including, for example, sodium hyaluronate (the sodium salt), potassium hyaluronate, magnesium hyaluronate, and calcium hyaluronate. Hyaluronic acid from a variety of sources may be used herein. For example, hyaluronic acid may be extracted from animal tissues, harvested as a product of bacterial fermentation, or produced in commercial quantities by bioprocess technology.

“Collagen” as described herein is a structural protein that is found in various connective tissues in the body. The collagen provided may come from any source. Without being limiting, the collagen may be marine collagen, bovine collagen, porcine collagen, human collagen, human collagen from cell culture or recombinant collagen, for example.

As described herein, “localized delivery” refers to the administration of the composition into or near the tissue in need of the therapeutic composition. In some embodiments of any one of each or any of the above- or below-mentioned embodiments, the macromolecular matrix is locally delivered into an area on the body of the patient having a volume deficit.

“Physically mixing” is to add in or fold in another matter for mixing. Without being limiting physically mixing may be performed by stirring in, stir bar, acoustic mixer, orbital mixer, for example.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A method of preparing a macromolecular matrix, the method comprising: providing a crosslinked hyaluronic acid, providing collagen; and physically mixing the collagen into the crosslinked hyaluronic acid, wherein the collagen is mixed homogeneously throughout the crosslinked hyaluronic acid, thereby forming the macromolecular matrix, wherein the macromolecular matrix comprises crosslinked hyaluronic acid and physically mixed in collagen.

Clause 2. The method of clause 1, wherein the collagen is provided in a soluble state as a solution.

Clause 3. The method of clause 1 or 2, wherein the collagen is provided as a solution in an acidic pH, wherein the collagen solution comprises a pH of about 2.0, about 2.5, about 3.0, about 3.5, about 4.0, about 4.5, about 5.0, about 5.5, about 6.0, about 6.5, about 7.0 or any pH within a range defined by any two aforementioned values, and wherein the collagen is soluble at the acidic pH.

Clause 4. The method of any one of clauses 1-3, wherein the collagen is provided as a solution in an acidic pH, wherein the collagen solution comprises a pH of about 2.0, about 2.5, about 3.0, about 3.5, about 4.0, about 4.5, about 5.0, about 5.5 or any pH within a range defined by any two aforementioned values, and wherein the collagen is soluble at the acidic pH.

Clause 5. The method of any one of clauses 1-3, wherein the collagen is provided as a solution at a neutral pH.

Clause 6. The method of any one of clauses 1-5, wherein the collagen and the crosslinked hyaluronic acid are physically mixed with a buffer.

Clause 7. The method of clause 6, wherein the buffer solution comprises PBS.

Clause 8. The method of any one of clauses 1-7, wherein the method further comprises neutralizing the macromolecular matrix to a pH of about 7, after mixing the collagen homogeneously throughout the crosslinked hyaluronic acid.

Clause 9. The method of any one of clauses 1-8, wherein the method further comprises neutralizing the collagen to a pH of about 7, prior to physically mixing the collagen with the crosslinked hyaluronic acid.

Clause 10. The method of clause 9, wherein neutralizing the collagen prior to physically mixing the collagen into the crosslinked hyaluronic acid causes the collagen to precipitate into fibrils or particles of collagen, wherein the fibrils or particles of collagen are further mixed into the crosslinked hyaluronic acid, wherein the fibrils or particles are mixed in homogeneously throughout the crosslinked hyaluronic acid.

Clause 11. The method of clause 1, wherein the collagen is provided as fibrillated collagen or collagen fibers.

Clause 12. The method of clause 11, wherein the collagen was prepared at a basic pH thereby producing the fibrillated collagen or collagen fibers.

Clause 13. The method of clause 11 or 12, wherein the collagen was prepared with at least one salt to obtain fibrillated collagen or collagen fibers.

Clause 14. The method of clause 13, wherein the at least one salt comprises a concentration of about 20 mM, about 50 mM, about 100 mM, about 150 mM, about 200 mM, about 250 mM, about 300 mM, about 350 mM, about 400 mM, about 450 mM, or about 500 mM, or any concentration in between a range defined by any two aforementioned values.

Clause 15. The method of clause 13 or 14, wherein the at least one salt comprises an anion wherein the anion comprises H2PO4-, SO42-, Cl— or SCN—.

Clause 16. The method of any one of clauses 13-15, wherein the at least one salt comprises NaCl, Na2SO4, or Li2SO4.

Clause 17. The method of any one of clauses 1-16, wherein the collagen is mixed homogeneously throughout the macromolecular matrix.

Clause 18. The method of any one of clauses 1-16, wherein the collagen is mixed evenly throughout the macromolecular matrix.

Clause 19. The method of any one of clauses 1-18, wherein the crosslinked hyaluronic acid comprises hyaluronic acid components comprising an average molecular weight of about 10,000 Daltons, about 20,000 Daltons, about 30,000 Daltons, about 40,000 Daltons, about 50,000 Daltons, about 60,000 Daltons, about 70,000 Daltons, about 80,000 Daltons, about 90,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,100,000 Daltons, about 1,200,000 Daltons, about 1,300,000 Daltons, about 1,400,000 Daltons, about 1,500,000 Daltons, about 1,600,000 Daltons, about 1,700,000 Daltons, about 1,800,000 Daltons, about 1,900,000 Daltons, about 2,000,000 Daltons, about 2,100,000 Daltons, about 2,200,000 Daltons, about 2,300,000 Daltons, about 2,400,000 Daltons, about 2,500,000 Daltons, about 2,600,000 Daltons, about 2,700,000 Daltons, about 2,800,000 Daltons, about 2,900,000 Daltons, about 3,000,000 Daltons, about 3,100,000 Daltons, about 3,200,000 Daltons, about 3,300,000 Daltons, about 3,400,000 Daltons, about 3,500,000 Daltons, about 3,600,000 Daltons, about 3,700,000 Daltons, about 3,800,000 Daltons, about 3,900,000 Daltons, about 4,000,000 Daltons, about 4,100,000 Daltons, about 4,200,000 Daltons, about 4,300,000 Daltons, about 4,400,000 Daltons, about 4,500,000 Daltons, about 4,600,000 Daltons, about 4,700,000 Daltons, about 4,800,000 Daltons, about 4,900,000 Daltons, about 5,000,000 Daltons, about 5,100,000 Daltons, about 5,200,000 Daltons, about 5,300,000 Daltons, about 5,400,000 Daltons, about 5,500,000 Daltons, about 5,600,000 Daltons, about 5,700,000 Daltons, about 5,800,000 Daltons, about 5,900,000 Daltons, about 6,000,000 Daltons, about 6,100,000 Daltons, about 6,200,000 Daltons, about 6,300,000 Daltons, about 6,400,000 Daltons, about 6,500,000 Daltons, about 6,600,000 Daltons, about 6,700,000 Daltons, about 6,800,000 Daltons, about 6,900,000 Daltons, about 7,000,000 Daltons, about 7,100,000 Daltons, about 7,200,000 Daltons, about 7,300,000 Daltons, about 7,400,000 Daltons, about 7,500,000 Daltons, about 7,600,000 Daltons, about 7,700,000 Daltons, about 7,800,000 Daltons, about 7,900,000 Daltons, about 8,000,000 Daltons, about 8,100,000 Daltons, about 8,200,000 Daltons, about 8,300,000 Daltons, about 8,400,000 Daltons, about 8,500,000 Daltons, about 8,600,000 Daltons, about 8,700,000 Daltons, about 8,800,000 Daltons, about 8,900,000 Daltons, about 9,000,000 Daltons, about 9,100,000 Daltons, about 9,200,000 Daltons, about 9,300,000 Daltons, about 9,400,000 Daltons, about 9,500,000 Daltons, about 9,600,000 Daltons, about 9,700,000 Daltons, about 9,800,000 Daltons, about 9,900,000 Daltons or about 10,000,000 Daltons or any molecular weight in between a range defined by any two aforementioned values.

Clause 20. The method of any one of clauses 1-19, wherein the crosslinked hyaluronic acid comprises a mixture of hyaluronic acid components comprising at least two different molecular weights.

Clause 21. The method of any one of clauses 1-20, wherein the method further comprises adding lidocaine to the macromolecular matrix.

Clause 22. The method of any one of clauses 1-21, wherein the lidocaine is at a concentration in between a range of about 0.15% (w/w) to about 0.45% (w/w) in the matrix.

Clause 23. The method of clause 21 or 22, wherein the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w)), about 0.39% (w/w), about 0.41% (w/w), about 0.43% (w/w), or about 0.45% (w/w) of the matrix or any concentration in between a range defined by any two aforementioned values.

Clause 24. The method of any one of clauses 21-23, wherein the lidocaine is at a concentration in between a range of about 0.27% (w/w) to about 0.33% (w/w) in the matrix.

Clause 25. The method of any one of clauses 1-24, wherein the matrix further comprises un-crosslinked HA.

Clause 26. The method of clause 25, wherein the un-crosslinked HA comprises a concentration of up to about 5% (w/w) within the matrix.

Clause 27. The method of clause 25 or 26, wherein the un-crosslinked HA comprises a concentration of about 0% (w/w), about 1% (w/w), about 2% (w/w), about 3% (w/w), about 4% (w/w), about 5% (w/w) in the matrix or any concentration in between a range defined by any two aforementioned values.

Clause 28. The method of any one of clauses 25-27, wherein the un-crosslinked HA comprises a concentration of about 1% (w/w) in the matrix.

Clause 29. The method of any one of clauses 25-27, wherein the un-crosslinked HA comprises a concentration of about 2% (w/w) in the matrix.

Clause 30. The method of any one of clauses 25-27, wherein the un-crosslinked HA comprises a concentration of about 5% (w/w) in the matrix.

Clause 31. The method of any one of clauses 1-30, wherein the macromolecular matrix has enhanced extrudability through a needle, wherein the needle comprises a gauge size of 27G, 28G, 29G, 30G, 31G or 32G.

Clause 32. The method of any one of clauses 1-31, wherein the macromolecular matrix is stable for at least 6 months.

Clause 33. The method of any one of clauses 1-32, wherein the macromolecular matrix is stable for about 6, about 9, about 12, about 15, about 18, about 21, about 24, about 27, about 30, about 33 or about 36 months or any amount of time in between a range defined by any two aforementioned values.

Clause 34. The method of any one of clauses 1-33, wherein the macromolecular matrix is stable at a temperature of about 4° C., about 5° C., about 10° C., about 15° C., about 20° C., about 25° C. or any temperature in between a range defined by any two aforementioned values.

Clause 35. The method of any one of clauses 1-34, wherein the macromolecular matrix is stable at about 4° C.

Clause 36. The method of any one of clauses 1-35, wherein the macromolecular matrix is stable at about 25° C.

Clause 37. The method of any one of clauses 1-36, wherein the macromolecular matrix has minimal degradation at about 3, about 6, about 9, about 12, about 15, about 18, about 21, about 24, about 27, about 30, about 33 or about 36 months or any amount of time in between a range defined by any two aforementioned values.

Clause 38. A method of preparing a macromolecular matrix, the method comprising: dissolving hyaluronic acid in an aqueous solution to form an aqueous pre-reaction solution and preparing a second solution wherein the second solution comprises: a) a water soluble carbodiimide; and an N-hydroxysuccinimide or an N-hydroxysulfosuccinimide; or b) 1,4-butanediol diglycidyl ether (BDDE) in a solution of sodium hyaluronate in the presence of sodium hydroxide; and adding the second solution to the aqueous pre-reaction solution to form a crosslinking reaction mixture; crosslinking the hyaluronic acid, wherein the crosslinking reaction mixture reacts by crosslinking the hyaluronic acid, thereby forming the crosslinked hyaluronic acid; and

providing collagen; and physically mixing the collagen into the crosslinked hyaluronic acid, thereby producing a macromolecular matrix comprising crosslinked hyaluronic acid and collagen, wherein the collagen is mixed homogeneously throughout the crosslinked hyaluronic acid.

Clause 39. The method of clause 38, wherein the second solution comprises the water soluble carbodiimide and the N-hydroxysuccinimide or the N- hydroxysulfosuccinimide, wherein the hyaluronic acid is crosslinked with a naturally occurring amine, thereby forming the crosslinked hyaluronic acid.

Clause 40. The method of clause 38 or 39, wherein the second solution comprises the water soluble carbodiimide and the N-hydroxysuccinimide or the N-hydroxysulfosuccinimide and wherein the crosslinking is performed in the presence of MES.

Clause 41. The method of clause 38, wherein the second solution comprises the 1,4-butanediol diglycidyl ether (BDDE) in a solution of sodium hyaluronate in the presence of sodium hydroxide, and wherein the BDDE is used to cross-link the hyaluronic acid using epoxide chemistry.

Clause 42. The method of any one of clauses 38-41, wherein the collagen is provided in a soluble state as a solution.

Clause 43. The method of any one of clauses 38-42, wherein physically mixing step is performed in the presence of a buffer.

Clause 44. The method of clause 43, wherein the buffer comprises PBS.

Clause 45. The method of any one of clauses 38-44, wherein the collagen is provided as a collagen solution, wherein the collagen solution comprises a pH of about 2.0, about 2.5, about 3.0, about 3.5, about 4.0, about 4.5, about 5.0, about 5.5, about 6.0, about 6.5, about 7.0 or any pH within a range defined by any two aforementioned values, and wherein the collagen is soluble at an acidic pH.

Clause 46. The method of any one of clauses 38-45, wherein the collagen is provided as a solution in an acidic pH, wherein the collagen solution comprises a pH of about 2.0, about 2.5, about 3.0, about 3.5, about 4.0, about 4.5, about 5.0, about 5.5, or any pH within a range defined by any two aforementioned values, and wherein the collagen is soluble at the acidic pH.

Clause 47. The method of any one of clauses 38-46, wherein the collagen is provided at an acidic to neutral pH.

Clause 48. The method of any one of clauses 38-46, further comprising neutralizing the macromolecular matrix to a pH of about 7, after mixing the collagen homogeneously throughout the crosslinked hyaluronic acid.

Clause 49. The method of any one of clauses 38-48, further comprising neutralizing the collagen to a pH of about 7, prior to physically mixing the collagen with the crosslinked hyaluronic acid, wherein neutralizing the collagen prior to physically mixing the collagen into the crosslinked hyaluronic acid causes the collagen to precipitate into fibrils or particles of collagen, wherein the fibrils or particles of collagen are further mixed into the crosslinked hyaluronic acid, wherein the fibrils or particles are mixed in homogeneously throughout the crosslinked hyaluronic acid.

Clause 50. The method of clause 49, wherein the collagen is provided as fibrillated collagen or collagen fibers.

Clause 51. The method of clause 50, wherein the collagen was prepared at a basic pH thereby producing the fibrillated collagen or collagen fibers.

Clause 52. The method of clause 50 or 51, wherein the collagen was prepared with at least one salt to obtain fibrillated collagen or collagen fibers.

Clause 53. The method of clause 52, wherein the at least one salt comprises a concentration of of about 20 mM, about 50 mM, about 100 mM, about 150 mM, about 200 mM, about 250 mM, about 300 mM, about 350 mM, about 400 mM, about 450 mM, or about 500 mM, or any concentration in between a range defined by any two aforementioned values.

Clause 54. The method of clause 52 or 53, wherein the at least one salt comprises an anion wherein the anion comprises H2PO4-, SO42-, Cl— or SCN—.

Clause 55. The method of any one of clauses 52-54, wherein the at least one salt comprises NaCl, Na2SO4, or Li2SO4.

Clause 56. The method of any one of clauses 38-55, wherein the hyaluronic acid comprises an average molecular weight of about 10,000 Daltons, about 20,000 Daltons, about 30,000 Daltons, 40,000 Daltons, about 50,000 Daltons, about 60,000 Daltons, about 70,000 Daltons, about 80,000 Daltons, about 90,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,100,000 Daltons, about 1,200,000 Daltons, about 1,300,000 Daltons, about 1,400,000 Daltons, about 1,500,000 Daltons, about 1,600,000 Daltons, about 1,700,000 Daltons, about 1,800,000 Daltons, about 1,900,000 Daltons, about 2,000,000 Daltons, about 2,100,000 Daltons, about 2,200,000 Daltons, about 2,300,000 Daltons, about 2,400,000 Daltons, about 2,500,000 Daltons, about 2,600,000 Daltons, about 2,700,000 Daltons, about 2,800,000 Daltons, about 2,900,000 Daltons, about 3,000,000 Daltons, about 3,100,000 Daltons, about 3,200,000 Daltons, about 3,300,000 Daltons, about 3,400,000 Daltons, about 3,500,000 Daltons, about 3,600,000 Daltons, about 3,700,000 Daltons, about 3,800,000 Daltons, about 3,900,000 Daltons, about 4,000,000 Daltons, about 4,100,000 Daltons, about 4,200,000 Daltons, about 4,300,000 Daltons, about 4,400,000 Daltons, about 4,500,000 Daltons, about 4,600,000 Daltons, about 4,700,000 Daltons, about 4,800,000 Daltons, about 4,900,000 Daltons, about 5,000,000 Daltons, about 5,100,000 Daltons, about 5,200,000 Daltons, about 5,300,000 Daltons, about 5,400,000 Daltons, about 5,500,000 Daltons, about 5,600,000 Daltons, about 5,700,000 Daltons, about 5,800,000 Daltons, about 5,900,000 Daltons, about 6,000,000 Daltons, about 6,100,000 Daltons, about 6,200,000 Daltons, about 6,300,000 Daltons, about 6,400,000 Daltons, about 6,500,000 Daltons, about 6,600,000 Daltons, about 6,700,000 Daltons, about 6,800,000 Daltons, about 6,900,000 Daltons, about 7,000,000 Daltons, about 7,100,000 Daltons, about 7,200,000 Daltons, about 7,300,000 Daltons, about 7,400,000 Daltons, about 7,500,000 Daltons, about 7,600,000 Daltons, about 7,700,000 Daltons, about 7,800,000 Daltons, about 7,900,000 Daltons, about 8,000,000 Daltons, about 8,100,000 Daltons, about 8,200,000 Daltons, about 8,300,000 Daltons, about 8,400,000 Daltons, about 8,500,000 Daltons, about 8,600,000 Daltons, about 8,700,000 Daltons, about 8,800,000 Daltons, about 8,900,000 Daltons, about 9,000,000 Daltons, about 9,100,000 Daltons, about 9,200,000 Daltons, about 9,300,000 Daltons, about 9,400,000 Daltons, about 9,500,000 Daltons, about 9,600,000 Daltons, about 9,700,000 Daltons, about 9,800,000 Daltons, about 9,900,000 Daltons or about 10,000,000 Daltons or any molecular weight in between a range defined by any two aforementioned values.

Clause 57. The method of any one of clauses 38-56, wherein the hyaluronic acid comprises a mixture of hyaluronic acid components comprising at least two different molecular weights.

Clause 58. The method of any one of clauses 38-57, wherein the dissolving step further comprises dissolving both hyaluronic acid and collagen in an aqueous solution to form an aqueous pre-reaction solution comprising both hyaluronic acid and collagen and wherein the crosslinking reaction reacts by crosslinking both the hyaluronic acid and collagen into an HA-Collagen conjugated gel, and wherein the mixing step comprises physically mixing the HA-Collagen conjugated gel with additional collagen, wherein the collagen is in a soluble state and wherein the collagen is in a solution, wherein the solution is in an acidic pH, thereby producing a macromolecular matrix comprising HA-Collagen conjugated gel with physically mixed in collagen.

Clause 59. The method of any one of clauses 38-58, wherein collagen fibrils or collagen precipitates are formed after the mixing step with collagen, and wherein the collagen fibrils or collagen precipitates are mixed homogenously within the crosslinked hyaluronic acid.

Clause 60. The method of any one of clauses 38-58, wherein collagen fibrils or collagen precipitates are formed during the mixing step with collagen, and wherein the collagen fibrils or collagen precipitates are mixed homogenously within the crosslinked hyaluronic acid

Clause 61. The method of any one of clauses 1-60, wherein the method further comprises adjusting an osmolarity of the macromolecular matrix after the collagen is mixed throughout the crosslinked hyaluronic acid.

Clause 62. The method of any one of clauses 1-61, wherein the mixing step is performed at room temperature.

Clause 63. The method of any one of clauses 1-61, wherein the mixing step is performed at about 4° C.

Clause 64. The method of any one of clauses 1-63, wherein the method is performed at about 4° C.

Clause 65. The method of any one of clauses 1-64, wherein the method further comprises purifying the crosslinked hyaluronic acid or HA-Collagen conjugated gel, wherein the purifying is performed prior to the mixing step with the collagen.

Clause 66. The method of clause 65, wherein the purifying is performed using dialysis purification.

Clause 67. The method of clause 65 or 66, wherein the pH of the crosslinking reaction mixture is adjusted after the crosslinking is complete, wherein adjusting the pH is performed prior to the purifying step, and wherein the pH is adjust to about 7.0, about 7.2, about 7.4 about 7.6 or any pH in between a range defined by any two aforementioned values.

Clause 68. The method of any one of clauses 65-67, wherein the purifying is performed at a range between about 2° C. and about 8° C.

Clause 69. The method of any one of clauses 65-68, wherein the purifying is performed at about 2° C., about 4° C., about 6° C., or about 8° C., or any temperature in between a range defined by any two aforementioned values.

Clause 70. The method of any one of clauses 65-69, wherein the method further comprises sterilizing the crosslinked hyaluronic acid or HA-Collagen conjugated gel prior to mixing with collagen, wherein the sterilizing step is performed after the purifying step.

Clause 71. The method of clause 70, wherein the sterilizing step comprises transferring the crosslinked hyaluronic acid or HA-Collagen conjugated gel into a container, for sterilization; and sterilizing the crosslinked hyaluronic acid or HA-Collagen conjugated gel.

Clause 72. The method of clause 71, wherein the container is a syringe.

Clause 73. The method of any one of clauses 70-72, wherein the sterilizing is performed by heat (dry heat, steam heat, moist heat sterilization) radiation (non-ionizing, UV), ionizing (particulate (beta rays, gamma rays, x-rays), electromagnetic (e-beam), filtration or terminal sterilization.

Clause 74. The method of any one of clauses 1-73, wherein the method further comprises dialyzing the crosslinked macromolecular matrix or HA-Collagen conjugated gel, wherein dialysis is performed through a membrane having a molecular weight cutoff in a range between 1000 Daltons to about 100,000 Daltons, and wherein the dialyzing is performed prior to sterilization.

Clause 75. The method of clause 74, wherein the dialysis is performed in a buffer.

Clause 76. The method of clause 75, wherein the buffer comprises phosphate buffered saline or a sodium phosphate buffer.

Clause 77. The method of any one of clauses 75 or 76, wherein the buffer further comprises NaCl, and/or KCl.

Clause 78. The method of any one of clauses 38-75, wherein the method further comprises raising the pH of the crosslinked hyaluronic acid or HA-Collagen conjugated gel to a neutral pH after the crosslinking reaction is complete, wherein the raising the pH is performed prior to a sterilizing step.

Clause 79. The method of any one of clauses 38-75, wherein the method further comprises raising the pH of the crosslinked hyaluronic acid or HA-Collagen conjugated gel to about 7.0, about 7.2 or about 7.4, or any pH in between a range defined by any two aforementioned values, after the crosslinking reaction is complete, wherein the raising the pH is performed prior to a sterilizing step.

Clause 80. The method of any one of clauses 38-78, wherein the second solution comprises the water soluble carbodiimide and the N-hydroxysuccinimide or an N-hydroxysulfosuccinimide, and wherein the adding and crosslinking step is performed at a temperature between about 4° C. and about 22° C.

Clause 81. The method of clause 80, wherein the crosslinking is performed at about 22° C.

Clause 82. The method of clause 80, wherein the crosslinking is performed at about 4° C.

Clause 83. The method of any one of clauses 38-78, wherein the second solution comprises the 1,4-butanediol diglycidyl ether (BDDE) in the solution of sodium hyaluronate in the presence of sodium hydroxide, and wherein the adding and crosslinking step is performed between about 45° C. and about 75° C.

Clause 84. The method of any one of clauses 38-83, wherein the second solution comprises the 1,4-butanediol diglycidyl ether (BDDE) in the solution of sodium hyaluronate in the presence of sodium hydroxide, and wherein the adding and crosslinking step is performed at a temperature of about 45° C., about 50° C., about 55° C., about 60° C., about 65° C., about 70° C., about 75° C., or any temperature in between a range defined by any two aforementioned values.

Clause 85. The method of any one of clauses 38-84, wherein the second solution comprises the 1,4-butanediol diglycidyl ether (BDDE) in the solution of sodium hyaluronate in the presence of sodium hydroxide, and wherein the adding and crosslinking step is performed at a temperature of about 50° C.

Clause 86. The method of any one of clauses 1-85, wherein the method comprises a neutralizing step performed after forming the macromolecular matrix comprising the crosslinked hyaluronic acid and collagen or after forming the HA-Collagen conjugated gel with physically mixed in collagen, wherein the neutralizing step comprises adjusting the macromolecular matrix to a physiological pH and physiological salt concentration.

Clause 87. The method of clause 86, wherein the neutralizing comprises adding a basic solution or buffered solution after the mixing step under aseptic conditions to adjust the pH.

Clause 88. The method of clause 86 or 87, wherein adjusting the pH and physiological salt concentration causes precipitation of the collagen into fibrils or particles, wherein the collagen fibrils or particles are distributed homogeneously in the macromolecular matrix.

Clause 89. The method of any one of clauses 38-88, wherein the method further comprises stopping the crosslinking step, wherein the stopping step comprises adding a base to the crosslinking reaction mixture to a pH of between about 8 and about 10 for at least 10 minutes and then adding an acidic solution to the crosslinking mixture until a pH of about 7 is reached.

Clause 90. The method of any one of clauses 38-89, wherein the naturally occurring amine is from lysine.

Clause 91. The method of any one of clauses 38-90, wherein the crosslinking reaction mixture comprises a pH between about 4.0 and about 10.0.

Clause 92. The method of clause 91, wherein the pH of the crosslinking reaction mixture is between about 4.0 and about 6.0.

Clause 93. The method of any one of clauses 38-78 or 83-92, wherein the second solution comprises 1,4-butanediol diglycidyl ether (BDDE) in a solution of sodium hyaluronate in the presence of sodium hydroxide, and wherein the crosslinking is performed under alkaline conditions.

Clause 94. The method of any one of clauses 38-93, wherein the pre-reaction solution comprises a salt, wherein the salt comprises a concentration of about 50 mM, about 75 mM, about 100 mM, about 125 mM, about 150 mM, about 175 mM, about 200 mM, about 225 mM, about 250 mM, about 275 mM, about 300 mM, 325 mM, about 350 mM, about 375 mM, about 400 mM, about 425 mM, about 450 mM, about 475 mM, about 500 mM or any concentration in a range defined by any two aforementioned values.

Clause 95. The method of any one of clauses 38-81, 86-92 or 94, wherein the water soluble carbodiimide is 1-ethyl-3-(3-dimethylaminopropyl)carbodiimide and wherein the water soluble carbodiimide is at a concentration of about 20 mM to about 300 mM in the crosslinking reaction mixture.

Clause 96. The method of clause 95, wherein the concentration of the water soluble carbodiimide is 1-ethyl-3-(3-dimethylaminopropyl)carbodiimide is at a concentration of about 20 mM, about 40 mM, about 60 mM, about 80 mM, about 100 mM, about 120 mM, about 140 mM, about 160 mM, about 180 mM, about 200 mM, about 220 mM, about 240 mM, about 260 mM, about 280 mM, about 300 mM or any concentration in between a range defined by any two aforementioned values.

Clause 97. The method of any one of clauses 38-81, 86-92 or 94-96, wherein the water soluble carbodiimide and hyaluronic acid is at a mole to mole ratio of water soluble carbodiimide: hyaluronic acid repeat unit between about 0.3 to about 3.0.

Clause 98. The method of clause 97, wherein the water soluble carbodiimide and hyaluronic acid is at a mole to mole ratio of water soluble carbodiimide: hyaluronic acid repeat unit of about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9 or about 3.0 or any mole to mole ratio in between a range defined by any two aforementioned values.

Clause 99. The method of any one of clauses 38-98, wherein the lysine and hyaluronic acid are at a mole:mole (lysine:HA repeat unit) ratio between about 0.01 to about 0.6.

Clause 100. The method of clause 99, wherein the lysine and hyaluronic acid are at a mole:mole (lysine:HA repeat unit) ratio of about 0.01, about 0.02, about 0.03, about 0.04, about 0.05, about 0.06, about 0.07, about 0.08, about 0.09, about 0.10, about 0.11, about 0.12, about 0.13, about 0.14, about 0.15, about 0.16, about 0.17, about 0.18, about 0.19, about 0.2, about 0.21, about 0.22, about 0.23, about 0.24, about 0.25, about 0.26, about 0.27, about 0.28, about 0.29, about 0.3, about 0.31, about 0.32, about 0.33, about 0.34, about 0.35, about 0.36, about 0.37, about 0.38, about 0.39, about 0.4, about 0.41, about 0.42, about 0.43, about 0.44, about 0.45, about 0.46, about 0.47, about 0.48, about 0.49, about 0.5, about 0.51, about 0.52, about 0.53, about 0.54, about 0.55, about 0.56, about 0.57, about 0.58, about 0.59, about 0.6 or any mole to mole ratio in between a range defined by any two aforementioned values.

Clause 101. The method of any one of clauses 38-100, wherein the hyaluronic acid in the pre-reaction solution hydrates for at least about 60 minutes prior to the adding the second solution.

Clause 102. The method of any one of clauses 38-101, wherein the crosslinking reaction mixture is performed for about 4 hours to about 24 hours.

Clause 103. The method of any one of clauses 38-102, wherein the method further comprises adding lidocaine to the macromolecular matrix.

Clause 104. The method of clause 103, wherein the lidocaine is at a concentration in between a range of about 0.15% (w/w) to about 0.45% (w/w) in the matrix.

Clause 105. The method of clause 103 or 104, wherein the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w), about 0.39% (w/w), about 0.41% (w/w), about 0.43% (w/w), or about 0.45% (w/w) of the matrix or any concentration in between a range defined by any two aforementioned values.

Clause 106. The method of any one of clauses 103-105, wherein the lidocaine is at a concentration in between a range of about 0.27% (w/w) to about 0.33% (w/w) in the matrix.

Clause 107. The method of any one of clauses 38-106, wherein the matrix further comprises un-crosslinked HA.

Clause 108. The method of clause 107, wherein the un-crosslinked HA comprises a concentration of up to about 5% (w/w) within the matrix.

Clause 109. The method of clause 107-108, wherein the un-crosslinked HA comprises a concentration of about 0% (w/w), about 1% (w/w), about 2% (w/w), about 3% (w/w), about 4% (w/w), about 5% (w/w) in the matrix or any concentration in between a range defined by any two aforementioned values.

Clause 110. The method of any one of clauses 107-109, wherein the un-crosslinked HA comprises a concentration of about 1% (w/w) in the matrix.

Clause 111. The method of any one of clauses 107-109, wherein the un-crosslinked HA comprises a concentration of about 2% (w/w) in the matrix.

Clause 112. The method of any one of clauses 107-109, wherein the un-crosslinked HA comprises a concentration of about 5% (w/w) in the matrix.

Clause 113. The method of any one of clauses 38-112, wherein the macromolecular matrix has enhanced extrudability through a needle, wherein the needle comprises a gauge size of 27G, 28G, 29G, 30G, 31G or 32G.

Clause 114. The method of any one of clauses 1-113, wherein the method further comprises concentrating the collagen prior to adding the collagen to the crosslinked HA.

Clause 115. A macromolecular matrix made by the method of any one of clauses 1-114.

Clause 116. The macromolecular matrix of clause 115, wherein the macromolecular matrix comprises crosslinked hyaluronic acid, wherein the collagen is physically mixed with crosslinked hyaluronic acid, and wherein the collagen is homogeneous throughout the macromolecular complex.

Clause 117. The macromolecular matrix of clause 115, wherein the macromolecular matrix comprises HA-Collagen conjugated gel, wherein the HA-Collagen conjugated gel also comprises physically mixed in collagen that is not crosslinked to the HA-Collagen conjugated gel.

Clause 118. A macromolecular matrix comprising: hyaluronic acid, wherein the hyaluronic acid is crosslinked; and collagen; wherein the collagen is physically mixed with the crosslinked hyaluronic acid.

Clause 119. A macromolecular matrix comprising: an HA-Collagen conjugated gel, wherein the HA-Collagen conjugated gel comprises hyaluronic acid crosslinked with collagen; and physically mixed in collagen, wherein the physically mixed in collagen is not crosslinked to the HA-Collagen conjugated gel, and wherein the physically mixed in collagen is mixed homogeneously within the HA-Collagen conjugated gel.

Clause 120. The macromolecular matrix of any one of clauses 115-119, wherein the hyaluronic acid is crosslinked with a naturally occurring amine.

Clause 121. The macromolecular matrix of clause 120, wherein the naturally occurring amine is from lysine.

Clause 122. The macromolecular matrix of any one of clauses 115-121, wherein the collagen is from bovine collagen, marine collagen, human collagen or porcine collagen.

Clause 123. The macromolecular matrix of any one of clauses 115-122, wherein the collagen is recombinant human collagen.

Clause 124. The macromolecular matrix of any one of clauses 115-123, wherein the macromolecular matrix comprises an elastic modulus (G′) of about 100 Pa, about 200 Pa, about 300 Pa, about 400 Pa, about 500 Pa, about 600 Pa, about 700 Pa, about 800 Pa, about 900 Pa, about 1000 Pa, about 1100 Pa, about 1200 Pa, about 1300 Pa, about 1400 Pa, about 1500 Pa, about 1600 Pa, about 1700 Pa, about 1800 Pa, about 1900 Pa, about 2000 Pa, about 2100 Pa, about 2200 Pa, about 2300 Pa, about 2400 Pa, about 2500 Pa, about 2600 Pa, about 2700 Pa, about 2800 Pa, about 2900 Pa, or about 3000 Pa or any value in between a range defined by any two aforementioned values.

Clause 125. The macromolecular matrix of any one of clauses 115-124, wherein the macromolecular matrix comprises a compression force value of about 10 gmf, about 20 gmf, about 30 gmf, about 40 gmf, about 50 gmf, about 60 gmf, about 70 gmf, about 80 gmf, about 90 gmf, about 100 gmf, about 110 gmf, about 120 gmf, about 130 gmf, about 140 gmf, about 150 gmf, about 160 gmf, about 170 gmf, about 180 gmf, about 190 gmf, about 200 gmf, about 210 gmf, about 220 gmf, about 230 gmf, about 240 gmf, about 250 gmf, about 260 gmf, about 270 gmf, about 280 gmf, about 290 gmf, about 300 gmf, about 310 gmf, about 320 gmf, about 330 gmf, about 340 gmf, about 350 gmf, about 360 gmf, about 370 gmf, about 380 gmf, about 390 gmf, about 400 gmf, about 410 gmf, about 420 gmf, about 430 gmf, about 440 gmf, about 450 gmf, about 460 gmf, about 470 gmf, about 480 gmf, about 490 gmf, about 500 gmf, about 510 gmf, about 520 gmf, about 530 gmf, about 540 gmf, about 550 gmf, about 560 gmf, about 570 gmf, about 580 gmf, about 590 gmf or about 600 gmf or any compression force value in between a range defined by any two aforementioned values.

Clause 126. The macromolecular matrix of any one of clauses 115-125, wherein the hyaluronic acid is at a concentration of about 2 mg/ml, about 4 mg/ml, about 6 mg/ml, about 8 mg/ml, about 10 mg/ml, about 12 mg/ml, about 14 mg/ml, about 16 mg/ml, about 18 mg/ml, about 20 mg/ml, about 22 mg/ml, about 24 mg/ml, about 26 mg/ml about 28 mg/ml, about 30 mg/ml, 32 mg/ml, about 34 mg/ml or about 36 mg/ml or any concentration in between a range defined by any two aforementioned values.

Clause 127. The macromolecular matrix of any one of clauses 115-126, having a weight ratio of hyaluronic acid to collagen at about 20:3, about 24:2.3, about 24:10, about 24:12, about 24:4, about 24:6, about 28:6 or about 28:11.

Clause 128. The macromolecular matrix of any one of clauses 115-127, wherein the crosslinked hyaluronic acid comprises hyaluronic acid components comprising an average molecular weight of about 10,000 Daltons, about 20,000 Daltons, about 30,000 Daltons, 40,000 Daltons, about 50,000 Daltons, about 60,000 Daltons, about 70,000 Daltons, about 80,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,100,000 Daltons, about 1,200,000 Daltons, about 1,300,000 Daltons, about 1,400,000 Daltons, about 1,500,000 Daltons, about 1,600,000 Daltons, about 1,700,000 Daltons, about 1,800,000 Daltons, about 1,900,000 Daltons, about 2,000,000 Daltons, about 2,100,000 Daltons, about 2,200,000 Daltons, about 2,300,000 Daltons, about 2,400,000 Daltons, about 2,500,000 Daltons, about 2,600,000 Daltons, about 2,700,000 Daltons, about 2,800,000 Daltons, about 2,900,000 Daltons, about 3,000,000 Daltons, about 3,100,000 Daltons, about 3,200,000 Daltons, about 3,300,000 Daltons, about 3,400,000 Daltons, about 3,500,000 Daltons, about 3,600,000 Daltons, about 3,700,000 Daltons, about 3,800,000 Daltons, about 3,900,000 Daltons, about 4,000,000 Daltons, about 4,100,000 Daltons, about 4,200,000 Daltons, about 4,300,000 Daltons, about 4,400,000 Daltons, about 4,500,000 Daltons, about 4,600,000 Daltons, about 4,700,000 Daltons, about 4,800,000 Daltons, about 4,900,000 Daltons, about 5,000,000 Daltons, about 5,100,000 Daltons, about 5,200,000 Daltons, about 5,300,000 Daltons, about 5,400,000 Daltons, about 5,500,000 Daltons, about 5,600,000 Daltons, about 5,700,000 Daltons, about 5,800,000 Daltons, about 5,900,000 Daltons, about 6,000,000 Daltons, about 6,100,000 Daltons, about 6,200,000 Daltons, about 6,300,000 Daltons, about 6,400,000 Daltons, about 6,500,000 Daltons, about 6,600,000 Daltons, about 6,700,000 Daltons, about 6,800,000 Daltons, about 6,900,000 Daltons, about 7,000,000 Daltons, about 7,100,000 Daltons, about 7,200,000 Daltons, about 7,300,000 Daltons, about 7,400,000 Daltons, about 7,500,000 Daltons, about 7,600,000 Daltons, about 7,700,000 Daltons, about 7,800,000 Daltons, about 7,900,000 Daltons, about 8,000,000 Daltons, about 8,100,000 Daltons, about 8,200,000 Daltons, about 8,300,000 Daltons, about 8,400,000 Daltons, about 8,500,000 Daltons, about 8,600,000 Daltons, about 8,700,000 Daltons, about 8,800,000 Daltons, about 8,900,000 Daltons, about 9,000,000 Daltons, about 9,100,000 Daltons, about 9,200,000 Daltons, about 9,300,000 Daltons, about 9,400,000 Daltons, about 9,500,000 Daltons, about 9,600,000 Daltons, about 9,700,000 Daltons, about 9,800,000 Daltons, about 9,900,000 Daltons or about 10,000,000 Daltons or any weight in between a range defined by any two aforementioned values.

Clause 129. The macromolecular matrix of clause 128, wherein the hyaluronic acid components comprises a mixture of hyaluronic acid components comprising at least two different molecular weights.

Clause 130. The macromolecular matrix of any one of clauses 115-129, wherein the hyaluronic acid comprises a mixture of hyaluronic acids, wherein the mixture comprises a 50:50 blend of HA comprising two different molecular weights.

Clause 131. The macromolecular matrix of any one of clauses 115-130, wherein the collagen comprises Type I collagen and/or Type III collagen.

Clause 132. The macromolecular matrix of any one of clauses 115-131, wherein the collagen is at a concentration of about 1 mg/ml, about 2 mg/ml, about 3 mg/ml, about 4 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml or about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml or any concentration in between a range defined by any two aforementioned values.

Clause 133. The macromolecular matrix of any one of clauses 115-132, wherein the macromolecular matrix comprises an osmolality between about 250 mOsm/kg and about 390 mOsm/kg.

Clause 134. The macromolecular matrix of any one of clauses 115-133, wherein the macromolecular matrix comprises an osmolality between about 250 mOsm/kg, about 275 mOsm/kg, about 300 mOsm/kg, about 325 mOsm/kg, or about 390 mOsm/kg or any osmolality in between a range defined by any two aforementioned values.

Clause 135. The macromolecular matrix of any one of clauses 115-134, wherein the hyaluronic acid is linear.

Clause 136. The macromolecular matrix of any one of clauses 115-135, wherein the macromolecular matrix comprises a viscous modulus (G″) of about 10 Pa, about 20 Pa, about 30 Pa, about 40 Pa, about 50 Pa, about 60 Pa, about 70 Pa, about 80 Pa, about 90 Pa, about 100 Pa, about 200 Pa, about 300 Pa, about 400 Pa, about 500 Pa, about 600 Pa, about 700 Pa, about 800 Pa, about 900 Pa or about 1000 Pa or any viscous modulus (G″) in between a range defined by any two aforementioned values.

Clause 137. The macromolecular matrix of any one of clauses 115-136, wherein the macromolecular matrix comprises a tan delta (G″/G′) of about 0.01, about 0.02, about 0.03, about 0.04, about 0.05, about 0.06, about 0.07, about 0.08, about 0.09, about 0.1, about 0.12, about 0.14, about 0.16, about 0.18, about 0.20, about 0.22, about 0.24, about 0.26, about 0.28, about 0.30, about 0.32, about 0.34, about 0.36, about 0.38, about 0.40, about 0.42, about 0.44, about 0.46, about 0.48, about 0.50 or any tan delta (G″/G′) in between a range defined by any two aforementioned values.

Clause 138. The macromolecular matrix of any one of clauses 115-137, wherein the macromolecular matrix comprises a pH of about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0 or any pH in a range defined by any two aforementioned values

Clause 139. The macromolecular matrix of any one of clauses 115-138, wherein the macromolecular matrix further comprises lidocaine.

Clause 140. The macromolecular matrix of clause 139, wherein the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w), about 0.39% (w/w), about 0.41% (w/w), about 0.43% (w/w), or about 0.45% (w/w) of the matrix or any concentration in between a range defined by any two aforementioned values.

Clause 141. The macromolecular matrix of any one of clauses 139 or 140, wherein the lidocaine is at a concentration in between a range of about 0.27% (w/w) to about 0.33% (w/w) in the matrix.

Clause 142. The macromolecular matrix of any one of clauses 115-141, wherein the matrix further comprises un-crosslinked HA.

Clause 143. The macromolecular matrix of clause 142, wherein the un-crosslinked HA comprises a concentration of up to about 5% (w/w) within the matrix.

Clause 144. The macromolecular matrix of clause 142-143, wherein the un-crosslinked HA comprises a concentration of about 0% (w/w), about 1% (w/w), about 2% (w/w), about 3% (w/w), about 4% (w/w), about 5% (w/w) in the matrix or any concentration in between a range defined by any two aforementioned values.

Clause 145. The macromolecular matrix of any one of clauses 142-144, wherein the un-crosslinked HA comprises a concentration of about 1% (w/w) in the matrix.

Clause 146. The macromolecular matrix of any one of clauses 142-144, wherein the un-crosslinked HA comprises a concentration of about 2% (w/w) in the matrix.

Clause 147. The macromolecular matrix of any one of clauses 142-144, wherein the un-crosslinked HA comprises a concentration of about 5% (w/w) in the matrix.

Clause 148. The macromolecular matrix of any one of clauses 115-147, wherein the macromolecular matrix has enhanced extrudability through a needle, wherein the needle comprises a gauge size of 27G, 28G, 29G, 30G, 31G or 32G.

Clause 149. A method of improving an aesthetic quality of an anatomic feature of a human being comprising: injecting a composition into a tissue of the human being to thereby improve the aesthetic quality of the anatomic feature; wherein the composition comprises the macromolecular matrix prepared by the method of any one of clauses 1-102 or the macromolecular matrix of any one of clauses 115-139.

Clause 150. A method of improving an aesthetic quality of an anatomic feature of a human being comprising: injecting a composition into a tissue of the human being to thereby improve the aesthetic quality of the anatomic feature; wherein the composition comprises a macromolecular matrix comprising: crosslinked hyaluronic acid; lysine; and collagen; wherein the collagen is physically mixed into the crosslinked hyaluronic acid.

Clause 151. The method of clause 150, wherein the composition further comprises lidocaine.

Clause 152. The method of clause 151, wherein the lidocaine is at a concentration in between a range of 0.15% (w/w) to 0.45% (w/w) in the matrix.

Clause 153. The method of clause 151 or 152, wherein the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w), about 0.39% (w/w), about 0.41% (w/w), about 0.43% (w/w), or about 0.45% (w/w) of the matrix or any concentration in between a range defined by any two aforementioned values.

Clause 154. The method of any one of clauses 151-153, wherein the lidocaine is at a concentration in between a range of about 0.27% (w/w) to about 0.33% (w/w) in the matrix.

Clause 155. The method of any one of clauses 150-154, wherein the composition further comprises un-crosslinked HA.

Clause 156. The method of clause 155, wherein the un-crosslinked HA comprises a concentration of up to about 5% (w/w) within the matrix.

Clause 157. The method of clause 155 or 156, wherein the un-crosslinked HA comprises a concentration of about 0% (w/w), about 1% (w/w), about 2% (w/w), about 3% (w/w), about 4% (w/w), about 5% (w/w) in the matrix, or any concentration in between a range defined by any two aforementioned values.

Clause 158. The method of any one of clauses 155-157, wherein the un-crosslinked HA comprises a concentration of about 1% (w/w) in the matrix.

Clause 159. The method of any one of clauses 155-157, wherein the un-crosslinked HA comprises a concentration of about 2% (w/w) in the matrix.

Clause 160. The method of any one of clauses 155-157, wherein the un-crosslinked HA comprises a concentration of about 5% (w/w) in the matrix.

Clause 161. The method of any one of clauses 150-160, wherein the crosslinked hyaluronic acid comprises hyaluronic acid components comprising an average molecular weight of about 10,000 Daltons, about 20,000 Daltons, about 30,000 Daltons, 40,000 Daltons, about 50,000 Daltons, about 60,000 Daltons, about 70,000 Daltons, about 80,000 Daltons, about100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,100,000 Daltons, about 1,200,000 Daltons, about 1,300,000 Daltons, about 1,400,000 Daltons, about 1,500,000 Daltons, about 1,600,000 Daltons, about 1,700,000 Daltons, about 1,800,000 Daltons, about 1,900,000 Daltons, about 2,000,000 Daltons, about 2,100,000 Daltons, about 2,200,000 Daltons, about 2,300,000 Daltons, about 2,400,000 Daltons, about 2,500,000 Daltons, about 2,600,000 Daltons, about 2,700,000 Daltons, about 2,800,000 Daltons, about 2,900,000 Daltons, about 3,000,000 Daltons, about 3,100,000 Daltons, about 3,200,000 Daltons, about 3,300,000 Daltons, about 3,400,000 Daltons, about 3,500,000 Daltons, about 3,600,000 Daltons, about 3,700,000 Daltons, about 3,800,000 Daltons, about 3,900,000 Daltons, about 4,000,000 Daltons, about 4,100,000 Daltons, about 4,200,000 Daltons, about 4,300,000 Daltons, about 4,400,000 Daltons, about 4,500,000 Daltons, about 4,600,000 Daltons, about 4,700,000 Daltons, about 4,800,000 Daltons, about 4,900,000 Daltons, about 5,000,000 Daltons, about 5,100,000 Daltons, about 5,200,000 Daltons, about 5,300,000 Daltons, about 5,400,000 Daltons, about 5,500,000 Daltons, about 5,600,000 Daltons, about 5,700,000 Daltons, about 5,800,000 Daltons, about 5,900,000 Daltons, about 6,000,000 Daltons, about 6,100,000 Daltons, about 6,200,000 Daltons, about 6,300,000 Daltons, about 6,400,000 Daltons, about 6,500,000 Daltons, about 6,600,000 Daltons, about 6,700,000 Daltons, about 6,800,000 Daltons, about 6,900,000 Daltons, about 7,000,000 Daltons, about 7,100,000 Daltons, about 7,200,000 Daltons, about 7,300,000 Daltons, about 7,400,000 Daltons, about 7,500,000 Daltons, about 7,600,000 Daltons, about 7,700,000 Daltons, about 7,800,000 Daltons, about 7,900,000 Daltons, about 8,000,000 Daltons, about 8,100,000 Daltons, about 8,200,000 Daltons, about 8,300,000 Daltons, about 8,400,000 Daltons, about 8,500,000 Daltons, about 8,600,000 Daltons, about 8,700,000 Daltons, about 8,800,000 Daltons, about 8,900,000 Daltons, about 9,000,000 Daltons, about 9,100,000 Daltons, about 9,200,000 Daltons, about 9,300,000 Daltons, about 9,400,000 Daltons, about 9,500,000 Daltons, about 9,600,000 Daltons, about 9,700,000 Daltons, about 9,800,000 Daltons, about 9,900,000 Daltons or about 10,000,000 Daltons or any weight in between a range defined by any two aforementioned values.

Clause 162. The method of any one of clauses 150-161, wherein the hyaluronic acid of the crosslinked hyaluronic acid comprises a mixture of hyaluronic acid components comprising at least two different molecular weights.

Clause 163. The method of any one of clauses 150 -162, wherein the collagen comprises collagen type I and/or collagen type III.

Clause 164. The method of any one of clauses 150-163, wherein the method does not cause a bluish discoloration at a site of injection.

Clause 165. The method of any one of clauses 150-164, wherein the composition has enhanced extrudability through a needle, wherein the needle comprises a gauge size of 27G, 28G, 29G, 30G, 31G or 32G.

Clause 166. A method of improving the appearance of a human being by injecting a composition into the tissue of a human being whereby the composition promotes cell infiltration and collagen deposition into the composition from the surrounding tissue into which it was injected, the method comprising: injecting a composition into a tissue of the human being to thereby improve the aesthetic quality of the anatomic feature; wherein the composition comprises a crosslinked macromolecular matrix comprising: hyaluronic acid; lysine; and collagen; wherein the hyaluronic acid comprises crosslinked hyaluronic acid that is physically mixed with the collagen; and wherein the tissue injected by the composition is shown to have tissue integration.

Clause 167. The method of clause 166, wherein the composition further comprises lidocaine.

Clause 168. The method of clause 166 or 167, wherein the hyaluronic acid component has an average molecular weight of about 10,000 Daltons, about 20,000 Daltons, about 30,000 Daltons, 40,000 Daltons, about 50,000 Daltons, about 60,000 Daltons, about 70,000 Daltons, about 80,000 Daltons, about100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,100,000 Daltons, about 1,200,000 Daltons, about 1,300,000 Daltons, about 1,400,000 Daltons, about 1,500,000 Daltons, about 1,600,000 Daltons, about 1,700,000 Daltons, about 1,800,000 Daltons, about 1,900,000 Daltons, about 2,000,000 Daltons, about 2,100,000 Daltons, about 2,200,000 Daltons, about 2,300,000 Daltons, about 2,400,000 Daltons, about 2,500,000 Daltons, about 2,600,000 Daltons, about 2,700,000 Daltons, about 2,800,000 Daltons, about 2,900,000 Daltons, about 3,000,000 Daltons, about 3,100,000 Daltons, about 3,200,000 Daltons, about 3,300,000 Daltons, about 3,400,000 Daltons, about 3,500,000 Daltons, about 3,600,000 Daltons, about 3,700,000 Daltons, about 3,800,000 Daltons, about 3,900,000 Daltons, about 4,000,000 Daltons, about 4,100,000 Daltons, about 4,200,000 Daltons, about 4,300,000 Daltons, about 4,400,000 Daltons, about 4,500,000 Daltons, about 4,600,000 Daltons, about 4,700,000 Daltons, about 4,800,000 Daltons, about 4,900,000 Daltons, about 5,000,000 Daltons, about 5,100,000 Daltons, about 5,200,000 Daltons, about 5,300,000 Daltons, about 5,400,000 Daltons, about 5,500,000 Daltons, about 5,600,000 Daltons, about 5,700,000 Daltons, about 5,800,000 Daltons, about 5,900,000 Daltons, about 6,000,000 Daltons, about 6,100,000 Daltons, about 6,200,000 Daltons, about 6,300,000 Daltons, about 6,400,000 Daltons, about 6,500,000 Daltons, about 6,600,000 Daltons, about 6,700,000 Daltons, about 6,800,000 Daltons, about 6,900,000 Daltons, about 7,000,000 Daltons, about 7,100,000 Daltons, about 7,200,000 Daltons, about 7,300,000 Daltons, about 7,400,000 Daltons, about 7,500,000 Daltons, about 7,600,000 Daltons, about 7,700,000 Daltons, about 7,800,000 Daltons, about 7,900,000 Daltons, about 8,000,000 Daltons, about 8,100,000 Daltons, about 8,200,000 Daltons, about 8,300,000 Daltons, about 8,400,000 Daltons, about 8,500,000 Daltons, about 8,600,000 Daltons, about 8,700,000 Daltons, about 8,800,000 Daltons, about 8,900,000 Daltons, about 9,000,000 Daltons, about 9,100,000 Daltons, about 9,200,000 Daltons, about 9,300,000 Daltons, about 9,400,000 Daltons, about 9,500,000 Daltons, about 9,600,000 Daltons, about 9,700,000 Daltons, about 9,800,000 Daltons, about 9,900,000 Daltons or about 10,000,000 Daltons or any weight in between a range defined by any two aforementioned values.

Clause 169. The method of any one of clauses 166-168, wherein the hyaluronic acid of the crosslinked hyaluronic acid comprises a mixture of hyaluronic acid components comprising at least two different molecular weights.

Clause 170. The method of any one of clauses 166-169, wherein the collagen comprises collagen type I and/or collagen type III.

Clause 171. The method of any one of clauses 166-170, wherein the tissue injected by the composition is shown to have tissue integration into the composition and collagen deposition within the composition by infiltrating cells from surrounding tissue.

Clause 172. The method of any one of clauses 166-171, wherein the tissue injected by the composition is shown to have tissue integration into the composition and collagen deposition within the composition after injecting the composition.

Clause 173. The method of any one of clauses 166-172, wherein the composition has enhanced extrudability through a needle, wherein the needle comprises a gauge size of 27G, 28G, 29G, 30G, 31G or 32G.

Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

### EXAMPLES

The following examples, including the experiments conducted and the results achieved, are provided for illustrative purposes only and are not to be construed as limiting the disclosure.

### Example 1—Hydrogel Preparation

To prepare the hydrogel formulations, first the HA dermal filler, porcine collagen, water and 10×PBS were mixed together at acidic pH. The 10×PBS was added to adjust the solution to isotonic and buffered conditions. The pH of the solution was then raised to —7.4 by adding 9.1 wt% NaOH and mixing a second time. The increase in pH initiated the self-assembly of the collagen network (Table 1). Some formulations were also autoclaved after mixing.

TABLE 1
Chemical composition, osmolality and pH of HA-Collagen hydrogels.

| | | | | HA | Collagen | Osmolality | |
|---|---|---|---|---|---|---|---|
| # | Collagen | Dermal Filler | Formulation Name | (mg/ml) | (mg/ml) | (mOsm/kg) | pH |
| 1 | Sunmax | NOA | 20NO Aa0CN AS | 20 | 0 | 278 | 7.33 |
| | | | 20NO Aa2CN AS | 20 | 2.3 | 286 | 7.52 |
| | | | 24NO Aa0CN AS | 24 | 0 | 278 | 7.14 |
| | | | 24NO Aa6CN AS | 24 | 6 | 302 | 7.66 |
| | | | 24NO Aa0CN AC* | 24 | 6 | 304 | 7.65 |
| | | | 28NO Aa0CN AS | 28 | 0 | 284 | 7.35 |
| | | | 28NO Aa6CN AS | 28 | 6 | 300 | 7.86 |
| 1 | Theracol | Formulation 1 | 6.5Form. 10TC AS | 6.5 | 0.0 | 290 | 7.49 |
| 2 | | | 6.5Form. 12TC AS | 6.5 | 2.0 | 285 | 7.44 |
| 3 | | | 6.5 Form. 18TC AS | 6.5 | 8.0 | 300 | 7.14 |
| 4 | | Formulation 2 | 12.7 Form. 20TC AS | 12.7 | 0.0 | 300 | 7.29 |
| 5 | | | 12.7 Form. 22TC AS | 12.7 | 2.0 | 302 | 7.29 |
| 6 | | | 12.7 Form. 24TC AS | 12.7 | 4.0 | 296 | 7.18 |

(NOA: lysine crosslinked HA gels; Form 1: Formulation 1 24 mg/ml mg/ml HA; Form 2: Formulation 2 20 mg/ml HA; AS—Aseptically mixed; AC—autoclaved; TC—11 mg/ml collagen; CN—Collagen; HA—hyaluronic acid; *autoclaved samples)

### Example 2—Opacity

With the addition of collagen, the hydrogels became white in appearance and progressively more opaque (FIG. 1). The self-assembly of collagen led to a physically crosslinked network of fibers that scatter light to give the gel an opaque appearance. As such, the opacity of the hydrogels provided an advantage over transparent HA-only hydrogels of not creating a Tyndall effect at the site of the injection in a patient.

### Example 3—Rheological Properties

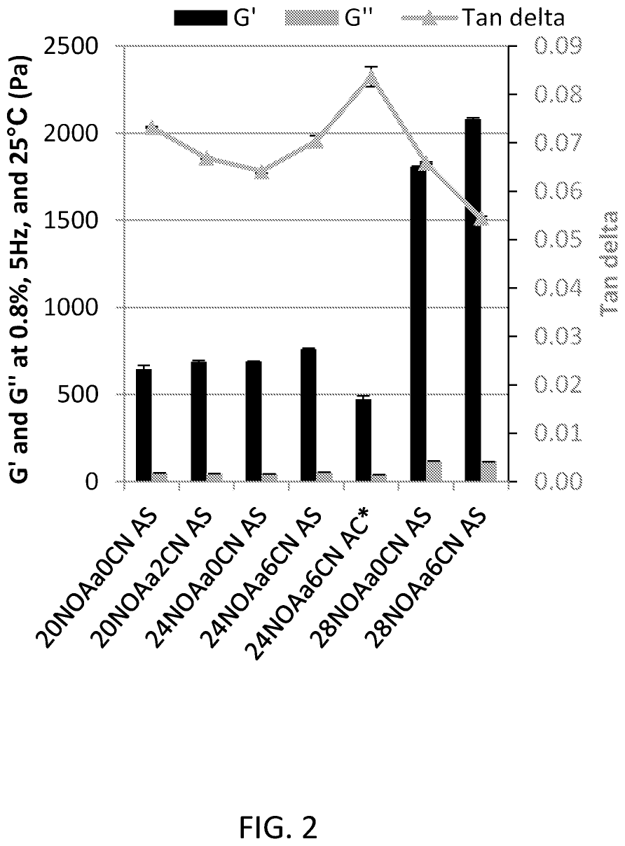

The rheology setup used to measure the viscoelasticity (G′, G″) of the HA/collagen hydrogels consisted of 25 mm diameter parallel plates set to a gap height of 1 mm. The measurement temperature was set to 25° C. with an oscillatory frequency of 5 Hz and 0.8% strain. The elastic modulus (G′) increased with increasing collagen and HA concentrations. The G′ value decreased after autoclaving in the presence of collagen but not in the absence of collagen (FIG. 2).

### Example 4—Swelling

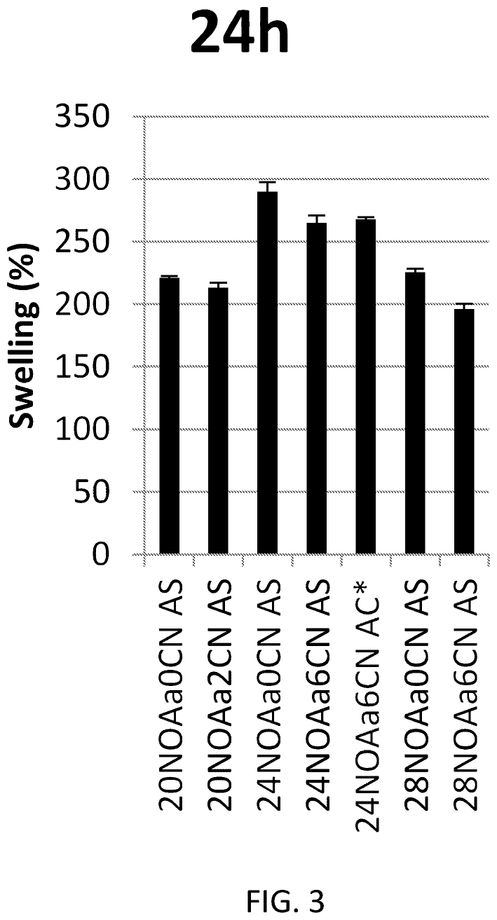

The swelling of each hydrogel formulation was quantified. Briefly, hydrogel samples (1 part) were mixed with phosphate buffered saline (PBS) (7 parts) using two connected syringes. Mixing was done for at least one hour. After 1 hour, the gel and PBS were transferred to one syringe and centrifuged (750 RCF, 10 min, room temperature). Polymer beads containing dye were added to the supernatant and the syringe was spun down again (50 RCF, 2 min, room temperature) to visualize the gel/supernatant interface. The change in gel volume relative to total volume was used to calculate the swelling of the hydrogel material (FIG. 3). Formulations with higher collagen concentrations demonstrated reduced swelling compared to similar gels without collagen.

### Example 5—Cohesivity

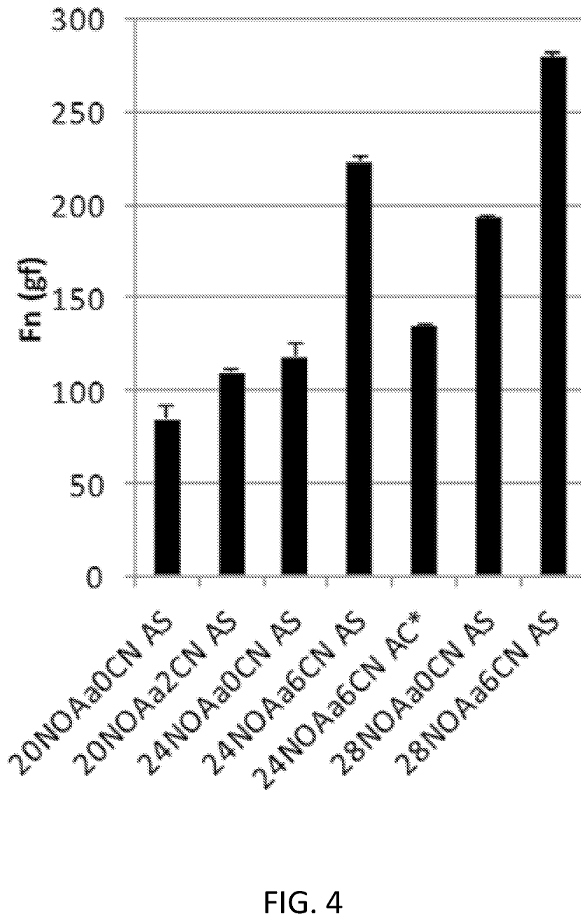

Cohesivity was quantified by measuring the compression force of the HA-Collagen materials, the difference in normal force as a parallel 25 mm diameter plate was lowered from 2.5 mm to 0.9 mm gap height at 0.8 mm/min. A gel with a higher compression force (change in normal force between 2.5 and 0.9 mm gap height) is considered to be more cohesive. Cohesivity increased with the addition of collagen and with higher concentration of HA (FIG. 4). Autoclaving formulations containing collagen resulted in cohesivity values similar to those formulations with similar HA concentrations, but without collagen.

### Example 6—Extrusion Forces

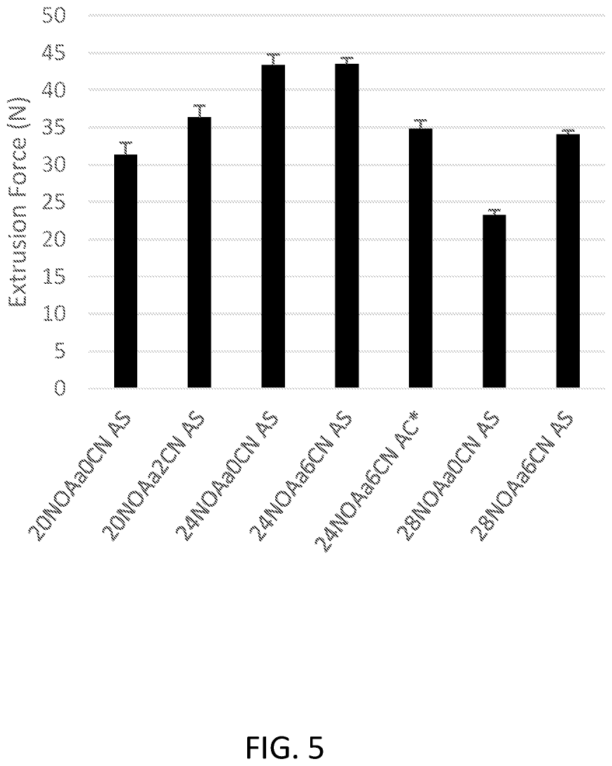

Extrusion force was measured using a 1 mL BD tuberculin syringe fitted with a 27G, ½″ needle at a speed of 100 mm/min. Extrusion force increased with collagen content (FIG. 5). After autoclaving, the extrusion force was found to be lower than for the same formulation before autoclave.

### Example 7—In Vivo Lift Capacity

The capacity of hydrogels to support tissue projection (lift) was evaluated in vivo with a subcutaneous implantation model in rats. 125 μL of hydrogel (n=10) was injected as a subcutaneous bolus on top of the skull. A clinical 3-D imaging system (Canfield Vectra) was used to generate 3-D reconstructions of the bolus over the course of 12 weeks. The mean height of the bolus was analyzed using medical imaging software (Canfield Mirror).

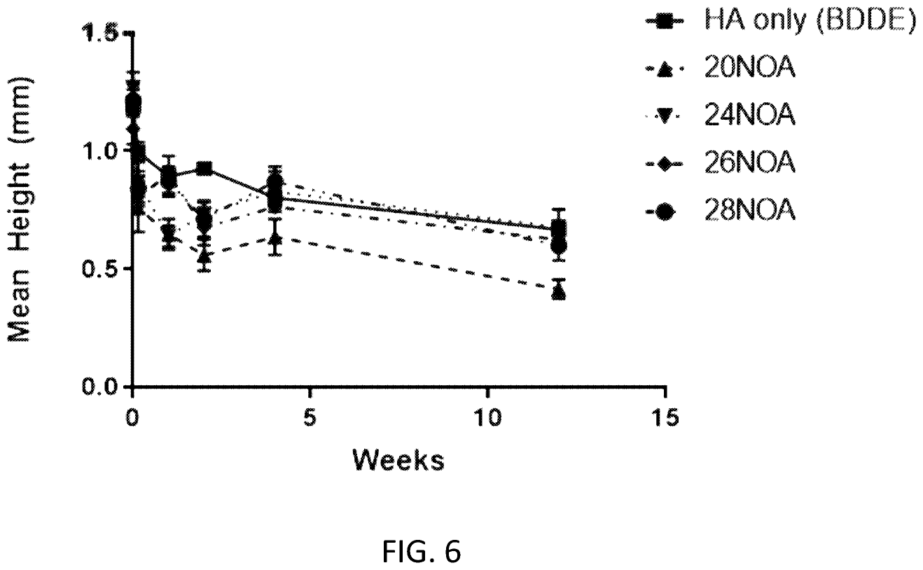

The in vivo lift capacity of a series of NOA crosslinked HA formulations (no collagen) and increasing [HA] measured between 4 and 12 weeks and showed a positive correlation with HA concentration from 20 mg/mL to 24 mg/mL (FIG. 6). Formulations with [HA] above 24 mg/mL did not demonstrate increased lift capacity. Furthermore, the lift capacity of the 24NOA gel is comparable to the HA only BDDE crosslinked gel.

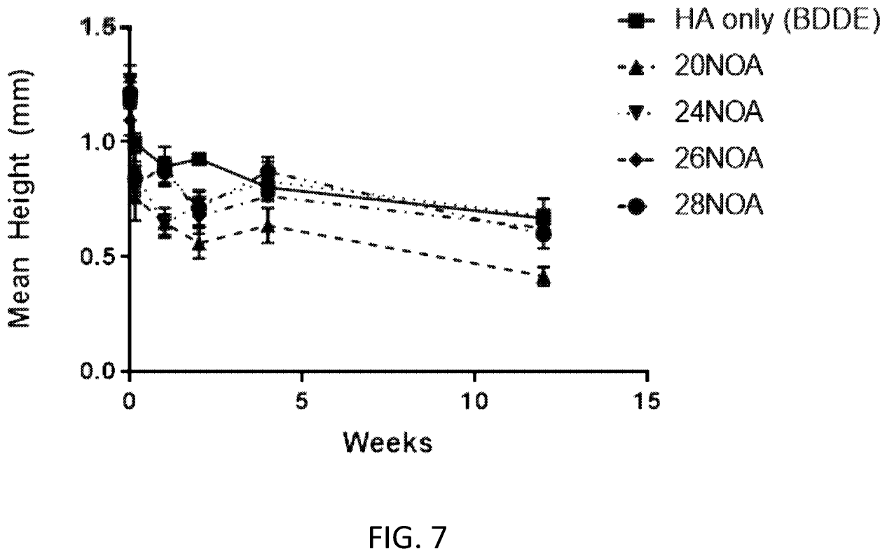

The in vivo lift capacity of a series of NOA crosslinked HA formulations with 20 mg/mL HA or 24 mg/mL and low concentrations of collagen (2.3 mg/mL to 3.0 mg/mL) measured between 4 and 12 weeks showed that mixing collagen into the HA gel does not impact lift capacity (FIG. 7). Furthermore, the lift capacity of the 24NOA2.3 gel is comparable to the HA only BDDE crosslinked gel.

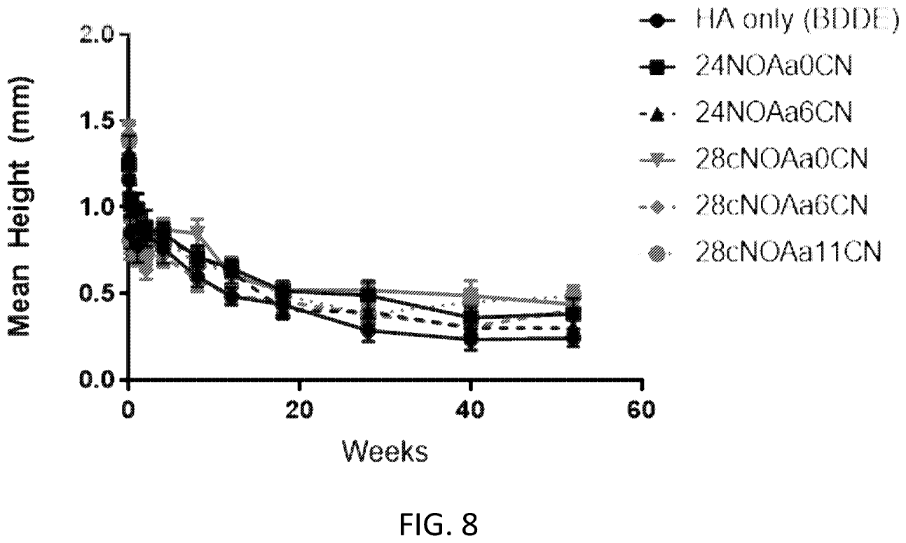

The in vivo lift capacity of a series of NOA crosslinked HA formulations with 24 mg/mL HA or 28 mg/mL and high concentrations of collagen (6 mg/mL to 11 mg/mL) measured between 4 and 52 weeks showed that gels crosslinked with NOA and mixed with collagen exhibit long term lift capacity as good or better than the HA only comparator (FIG. 8). Furthermore, the lift capacity of the NOA crosslinked HA gels or HA-Collagen gels is similar to the BDDE crosslinked HA only gel.

### Example 8—In Vitro Testing of Cell Response to Hydrogels.

Viability of fibroblast cells in close contact with HA-Collagen hydrogels was quantified. This assay measures cell activity by monitoring the reduction of a tetrazolium salt into a pigmented formazan product by cellular enzymes. Cells with greater viability show greater light absorbance at a particular wavelength, whereas cells with lower viability/proliferation show less absorbance. The absorbance can be measured and compared against a positive control (tissue culture plate) and a negative control (HA-only hydrogels) to give a relative proliferation value. Hydrogels that support greater cell viability may be expected to induce more cell infiltration into the gel with those cells depositing ECM within the gel matrix and may be beneficial for in vivo tissue integration into hydrogel depots. Conversely, those formulations which result in lower cell proliferation values would behave more inertly and allow for less tissue infiltration and integration.

100 μL of hydrogel (n=3) was layered on the bottom of a 24-well cell culture plate with a low adhesion surface coating and placed in a humidified incubator at 37° C. for 30 minutes. 50,000 adult human dermal fibroblasts in 500 μL of cell culture medium were added on top of the hydrogel beds and incubated 37° C. After 48 hours of incubation, 250 μL of XTT reagent was added to each well and incubated at 37° C. for 4 hours. The plate was then spun at 300xg for 5 minutes and 200 μL of supernatant from each well was transferred to wells of 96-well filter plate with a 20 μm mesh. The filter plate containing the XTT supernatant was spun at 300×g for 5 minutes. 100 μL of filtered supernatant from each well was transferred to a clean 96-well plate (black walls, clear bottom) and the absorbance of the supernatant was read on a microplate reader (450 nm with 630 nm background correction). The data was normalized to the XTT cell viability of fibroblasts cultured on the positive control Tissue Culture Polystyrene (TCPS).

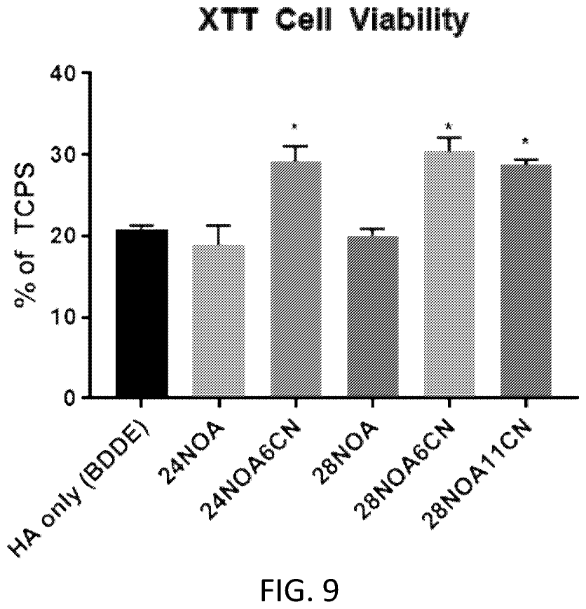

It was found that cell activity was higher for formulations with collagen mixed into the HA matrix than formulations without collagen (FIG. 9). For example, hydrogels composed of HA only (BDDE crosslinked or NOA crosslinked) exhibited cell viability values of 18.9% to 21%. Hydrogels with the addition of 6 mg/mL or 11 mg/mL of collagen exhibited cell viability values of 28.7% (28NOA11CN), 29.2% (24NOA6CN) or 30.4% (28NOA6CN).

In addition to the cell activity assays described above, a cell morphology analysis was conducted to better understand the effect of certain formulations on cell size, shape, and cytoskeleton organization. The actin filament alignment index and morphology of fibroblast cells cultured on HA-only or HA/collagen crosslinked hydrogels was imaged and quantified. Increased actin filament alignment may correlate to the increased adhesion of cells to their substrate. Increased length to width ratios correlates to increased cell spreading on a substrate. Hydrogels that support greater cell adhesion and spreading would be expected to induce more cell infiltration into the gel, with those cells depositing ECM within the gel matrix. Increased cell infiltration and ECM deposition could be beneficial for in vivo tissue integration into hydrogel depots. Conversely, those formulations which result in lower cell adhesion and spreading values would behave more inertly and allow for less tissue infiltration and integration.

In a typical procedure, hydrogels (n=3) and human dermal fibroblasts in cell culture medium were added to a 96-well cell culture plate with a low adhesion surface coating. After 48 hours of incubation, the cells were fixed in formalin and stained with Hoechst, WGA-488, and Alexa Fluor-Phalloidin. The wells were imaged with a confocal microscope and actin filament alignment (phalloidin) and cell morphology (WGA-488) were analyzed using image analysis software.

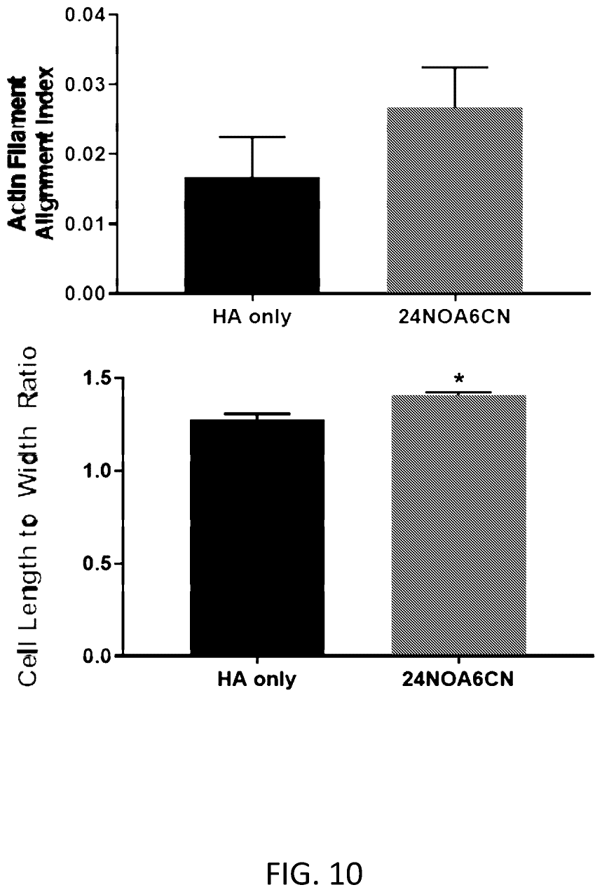

Collagen containing hydrogels exhibit improved cell adhesion and spreading on the hydrogel material (FIG. 10). The actin filament alignment index trends higher for the 24NOA6CN gel (0.026) compared to HA only (0.016). The cell length to width ratio (cell spreading) is significantly higher on the 24NOA6CN hydrogel (1.41) than HA only hydrogel (1.28).

The cell morphology analysis correlated well with the XTT cell activity assay in that the 24NOA6CN formulation which demonstrated a higher activity than HA only gel in the activity assay also showed evidence of enhanced cell adhesion and spreading in the morphology assay.

### Example 9—In Vivo Tissue Integration

In vivo tissue integration for a range of formulations was assessed using a subcutaneous implantation model in rats. In a typical procedure, 125 μL of hydrogel was delivered as a subcutaneous bolus on the dorsal aspect of the rat. After 4 or 12 weeks the bolus was explanted, fixed in formalin, and embedded in paraffin for histology. Tissue sections were stained for hematoxylin & eosin (H&E) and colloidal iron. Immunohistochemical staining for rat Collagen Type I was performed with an antibody that does not react with the exogenous porcine collagen.

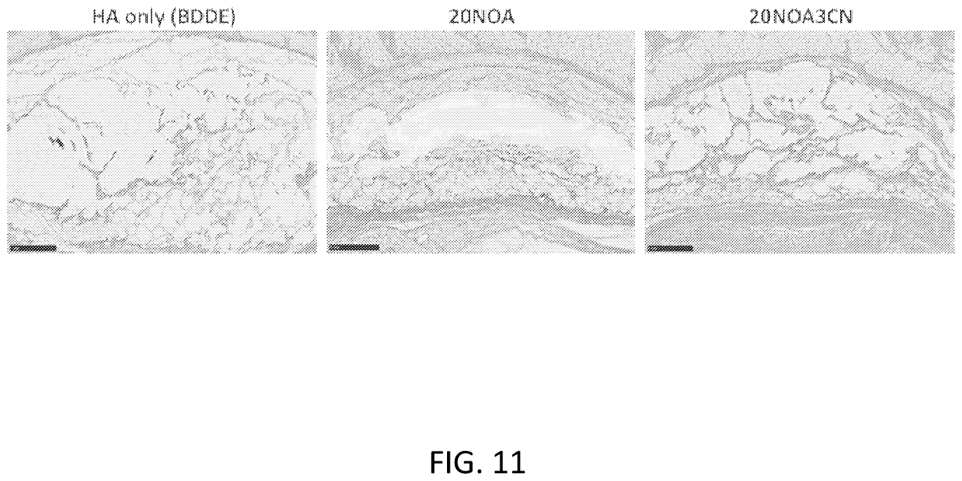

Formulations with 20 mg/mL HA (20NOA) and 3 mg/mL added collagen demonstrated enhanced cellular infiltration and tissue integration into the injected filler after 4 weeks, as judged by new host collagen deposition by fibroblast cells found in the injected hydrogel bolus (FIG. 11).

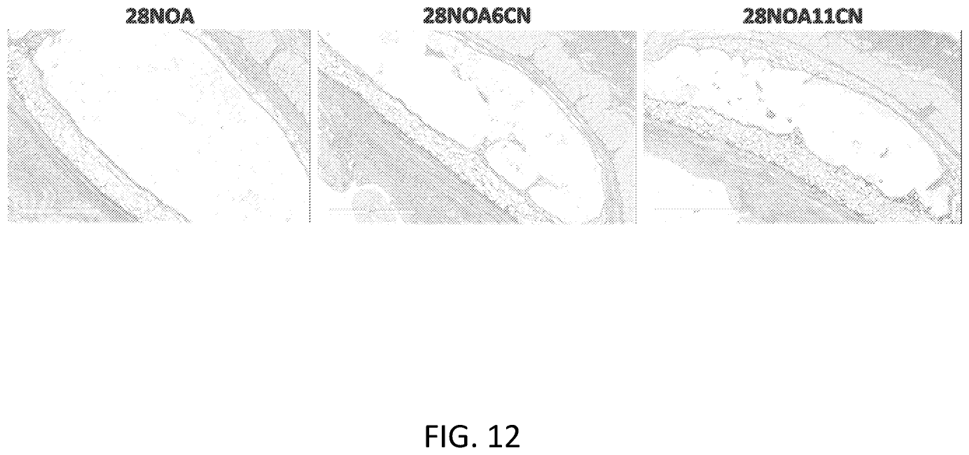

Formulations with 28 mg/mL HA (28NOA) exhibited encapsulation of the interior of the hydrogel bolus after 4 weeks (FIG. 12). The addition of up to 11 mg/mL collagen improved the tissue integration/collagen deposition around the periphery of the hydrogel bolus but did not prevent encapsulation. This may indicate that there is an upper limit to the HA concentration which may prevent tissue integration.

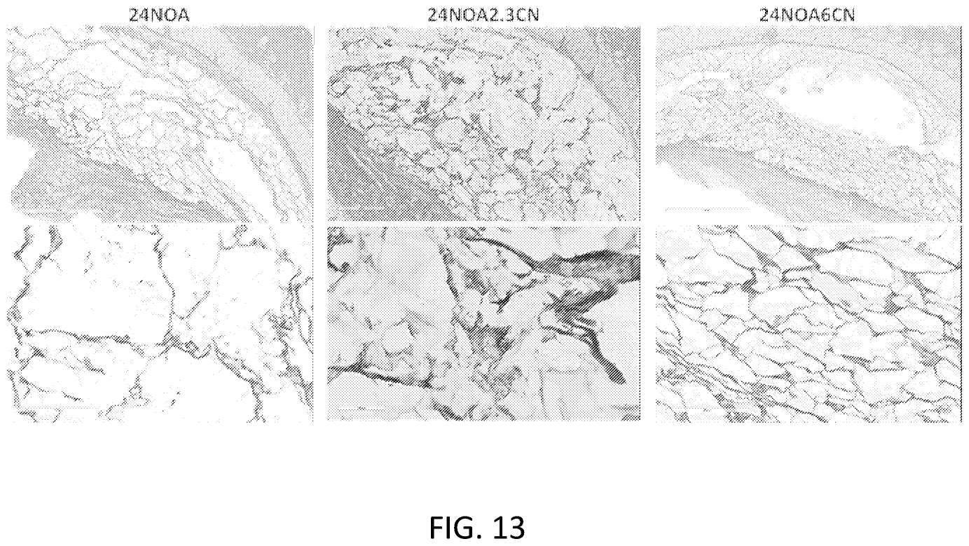

Formulations with 24 mg/mL HA (24NOA) exhibited tissue ingrowth and collagen deposition throughout most of the hydrogel bolus after 4 weeks (FIG. 13). The addition of collagen appeared to improve tissue integration in a dose dependent fashion. The 24NOA6CN gel exhibited robust new collagen deposition surrounding the hydrogel particles.

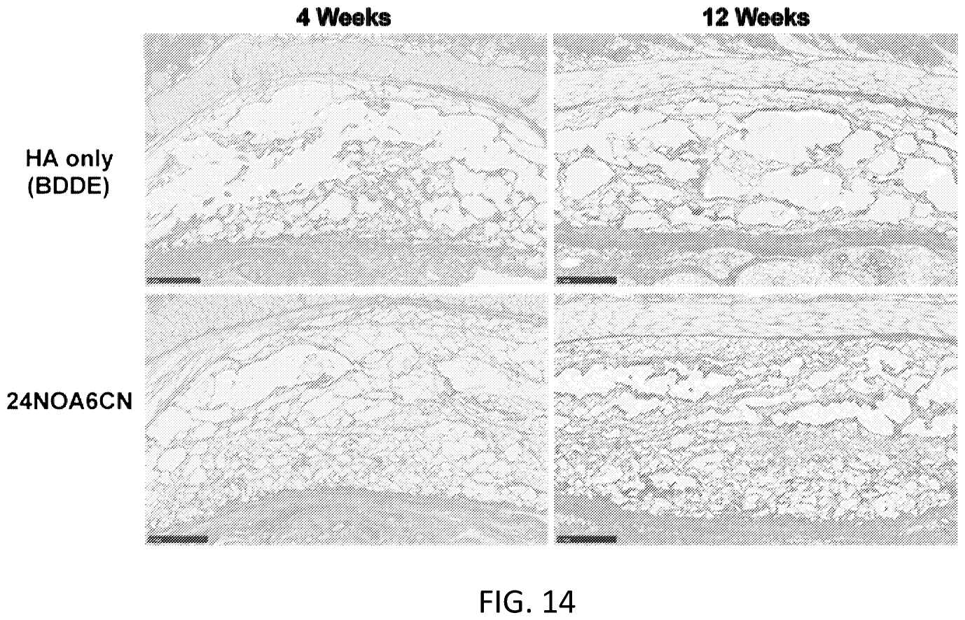

Formulations with 24 mg/mL HA and 6 mg/mL collagen (24NOA6CN) exhibited improving tissue integration/collagen deposition from 4 to 12 weeks of subcutaneous implantation (FIG. 14). The tissue integration of the 24NOA6CN formulation appeared substantially improved over the BDDE crosslinked HA only gel. Importantly, this mixed gel formulation (24NOA6CN) also exhibited lift capacity similar to a high lift BDDE crosslinked HA only gel. The 24NOA6CN formulation demonstrated a combination of lift capacity suitable for the correction of volume loss and enhanced tissue integration. This may result in the clinical benefit of initial volume correction followed by a natural look/feel and increased duration of effect.

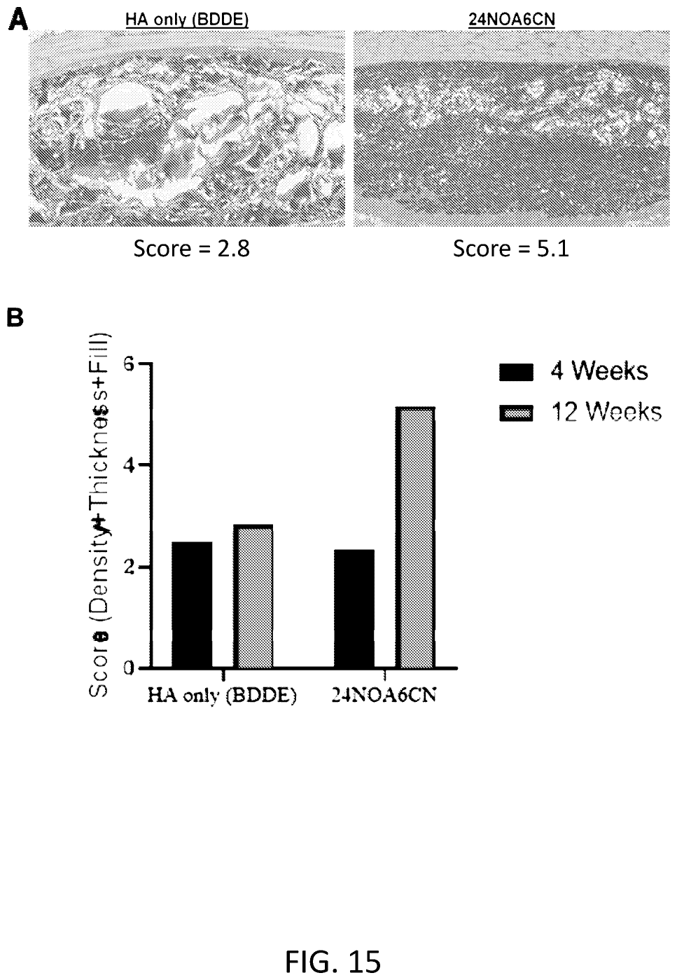

A semi-quantitative histopathological scoring system was developed to evaluate the tissue integration of hydrogel boluses following subcutaneous injection in rats. Briefly, a blinded pathologist scored the density of tissue in-growth (0=worst, 5=best), thickness of tissue bands (0=worst, 5=best), and tissue fill of the bolus (−5=no fill, 0=bolus filled with tissue) based on H&E stained sections. These three scores were combined producing a total score of 10 for the most highly integrated formulation and −5 for the least integration formulation. The tissue integration of the 24NOA6CN gel was quantified using this scoring system and compared to BDDE crosslinked HA only gel (FIG. 15). The integration of 24NOA6CN was similar to the HA only gel at 4 weeks (2.33 and 2.5, respectively). The integration of the 24NOA6CN gel scored higher than the HA only gel after 12 weeks (5.17 vs 2.83, respectively). It is also important to note that the integration of the 24NOA6CN gel improved over time while the HA only gel remained essentially the same. This may indicate an improving effect over time with HA-Collagen mixed gels.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those of skill within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

In addition, where features or aspects of the disclosure are described in terms of Markush groups, those skilled in the art will recognize that the disclosure is also thereby described in terms of any individual member or subgroup of members of the Markush group.

Any of the features of an embodiment of the first through sixth aspects is applicable to all aspects and embodiments identified herein. Moreover, any of the features of an embodiment of the first through sixth aspects is independently combinable, partly or wholly with other embodiments described herein in any way, e.g., one, two, or three or more embodiments may be combinable in whole or in part. Further, any of the features of an embodiment of the first through ninth aspects may be made optional to other aspects or embodiments.

What is claimed is:

1. A method of preparing a macromolecular matrix, the method comprising:
providing a crosslinked hyaluronic acid,
providing collagen; and
physically mixing the collagen into the crosslinked hyaluronic acid, wherein the collagen is mixed homogeneously throughout the crosslinked hyaluronic acid, thereby forming the macromolecular matrix, wherein the macromolecular matrix comprises crosslinked hyaluronic acid and physically mixed in collagen.

2. The method of claim 1, wherein the collagen is provided in a soluble state as a solution.

3. The method of claim 1 or 2, wherein the collagen is provided as a solution in an acidic pH, wherein the collagen solution comprises a pH of about 2.0, about 2.5, about 3.0, about 3.5, about 4.0, about 4.5, about 5.0, about 5.5, about 6.0, about 6.5, about 7.0 or any pH within a range defined by any two aforementioned values, and wherein the collagen is soluble at the acidic pH.

4. The method of any one of claims 1-3, wherein the collagen is provided as a solution in an acidic pH, wherein the collagen solution comprises a pH of about 2.0, about 2.5, about 3.0, about 3.5, about 4.0, about 4.5, about 5.0, about 5.5 or any pH within a range defined by any two aforementioned values, and wherein the collagen is soluble at the acidic pH.

5. The method of any one of claims 1-3, wherein the collagen is provided as a solution at a neutral pH.

6. The method of any one of claims 1-5, wherein the collagen and the crosslinked hyaluronic acid are physically mixed with a buffer.

7. The method of claim 6, wherein the buffer solution comprises PBS.

8. The method of any one of claims 1-7, wherein the method further comprises neutralizing the macromolecular matrix to a pH of about 7, after mixing the collagen homogeneously throughout the crosslinked hyaluronic acid.

9. The method of any one of claims 1-8, wherein the method further comprises neutralizing the collagen to a pH of about 7, prior to physically mixing the collagen with the crosslinked hyaluronic acid.

10. The method of claim 9, wherein neutralizing the collagen prior to physically mixing the collagen into the crosslinked hyaluronic acid causes the collagen to precipitate into fibrils or particles of collagen, wherein the fibrils or particles of collagen are further mixed into the crosslinked hyaluronic acid, wherein the fibrils or particles are mixed in homogeneously throughout the crosslinked hyaluronic acid.

11. The method of claim 1, wherein the collagen is provided as fibrillated collagen or collagen fibers.

12. The method of claim 11, wherein the collagen was prepared at a basic pH thereby producing the fibrillated collagen or collagen fibers.

13. The method of claim 11 or 12, wherein the collagen was prepared with at least one salt to obtain fibrillated collagen or collagen fibers.

14. The method of claim 13, wherein the at least one salt comprises a concentration of about 20 mM, about 50 mM, about 100 mM, about 150 mM, about 200 mM, about 250 mM, about 300 mM, about 350 mM, about 400 mM, about 450 mM, or about 500 mM, or any concentration in between a range defined by any two aforementioned values.

15. The method of claim 13 or 14, wherein the at least one salt comprises an anion wherein the anion comprises H₂PO⁴⁻, SO₄^{2″}, Cl⁻ or SCN⁻.

16. The method of any one of claims 13-15, wherein the at least one salt comprises NaCl, Na₂SO₄, or Li₂SO₄.

17. The method of any one of claims 1-16, wherein the collagen is mixed homogeneously throughout the macromolecular matrix.

18. The method of any one of claims 1-16, wherein the collagen is mixed evenly throughout the macromolecular matrix.

19. The method of any one of claims 1-18, wherein the crosslinked hyaluronic acid comprises hyaluronic acid components comprising an average molecular weight of about 10,000 Daltons, about 20,000 Daltons, about 30,000 Daltons, about 40,000 Daltons, about 50,000 Daltons, about 60,000 Daltons, about 70,000 Daltons, about 80,000 Daltons, about 90,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,100,000 Daltons, about 1,200,000 Daltons, about 1,300,000 Daltons, about 1,400,000 Daltons, about 1,500,000 Daltons, about 1,600,000 Daltons, about 1,700,000 Daltons, about 1,800,000 Daltons, about 1,900,000 Daltons, about 2,000,000 Daltons, about 2,100,000 Daltons, about 2,200,000 Daltons, about 2,300,000 Daltons, about 2,400,000 Daltons, about 2,500,000 Daltons, about 2,600,000 Daltons, about 2,700,000 Daltons, about 2,800,000 Daltons, about 2,900,000 Daltons, about 3,000,000 Daltons, about 3,100,000 Daltons, about 3,200,000 Daltons, about 3,300,000 Daltons, about 3,400,000 Daltons, about 3,500,000 Daltons, about 3,600,000 Daltons, about 3,700,000 Daltons, about 3,800,000 Daltons, about 3,900,000 Daltons, about 4,000,000 Daltons, about 4,100,000 Daltons, about 4,200,000 Daltons, about 4,300,000 Daltons, about 4,400,000 Daltons, about 4,500,000 Daltons, about 4,600,000 Daltons, about 4,700,000 Daltons, about 4,800,000 Daltons, about 4,900,000 Daltons, about 5,000,000 Daltons, about 5,100,000 Daltons, about 5,200,000 Daltons, about 5,300,000 Daltons, about 5,400,000 Daltons, about 5,500,000 Daltons, about 5,600,000 Daltons, about 5,700,000 Daltons, about 5,800,000 Daltons, about 5,900,000 Daltons, about 6,000,000 Daltons, about 6,100,000 Daltons, about 6,200,000 Daltons, about 6,300,000 Daltons, about 6,400,000 Daltons, about 6,500,000 Daltons, about 6,600,000 Daltons, about 6,700,000 Daltons, about 6,800,000 Daltons, about 6,900,000 Daltons, about 7,000,000 Daltons, about 7,100,000 Daltons, about 7,200,000 Daltons, about 7,300,000 Daltons, about 7,400,000 Daltons, about 7,500,000 Daltons, about 7,600,000 Daltons, about 7,700,000 Daltons, about 7,800,000 Daltons, about 7,900,000 Daltons, about 8,000,000 Daltons, about 8,100,000 Daltons, about 8,200,000 Daltons, about 8,300,000 Daltons, about 8,400,000 Daltons, about 8,500,000 Daltons, about 8,600,000 Daltons, about 8,700,000 Daltons, about 8,800,000 Daltons, about 8,900,000 Daltons, about 9,000,000 Daltons, about 9,100,000 Daltons, about 9,200,000 Daltons, about 9,300,000 Daltons, about 9,400,000 Daltons, about 9,500,000 Daltons, about 9,600,000 Daltons, about 9,700,000 Daltons, about 9,800,000 Daltons, about 9,900,000 Daltons or about 10,000,000 Daltons or any molecular weight in between a range defined by any two aforementioned values.

20. The method of any one of claims 1-19, wherein the crosslinked hyaluronic acid comprises a mixture of hyaluronic acid components comprising at least two different molecular weights.

21. The method of any one of claims 1-20, wherein the method further comprises adding lidocaine to the macromolecular matrix.

22. The method of any one of claims 1-21, wherein the lidocaine is at a concentration in between a range of about 0.15% (w/w) to about 0.45% (w/w) in the matrix.

23. The method of claim 21 or 22, wherein the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w)), about 0.39% (w/w), about 0.41% (w/w), about 0.43% (w/w), or about 0.45% (w/w) of the matrix or any concentration in between a range defined by any two aforementioned values.

24. The method of any one of claims 21-23, wherein the lidocaine is at a concentration in between a range of about 0.27% (w/w) to about 0.33% (w/w) in the matrix.

25. The method of any one of claims 1-24, wherein the matrix further comprises un-crosslinked HA.

26. The method of claim 25, wherein the un-crosslinked HA comprises a concentration of up to about 5% (w/w) within the matrix.

27. The method of claim 25 or 26, wherein the un-crosslinked HA comprises a concentration of about 0% (w/w), about 1% (w/w), about 2% (w/w), about 3% (w/w), about 4% (w/w), about 5% (w/w) in the matrix or any concentration in between a range defined by any two aforementioned values.

28. The method of any one of claims 25-27, wherein the un-crosslinked HA comprises a concentration of about 1% (w/w) in the matrix.

29. The method of any one of claims 25-27, wherein the un-crosslinked HA comprises a concentration of about 2% (w/w) in the matrix.

30. The method of any one of claims 25-27, wherein the un-crosslinked HA comprises a concentration of about 5% (w/w) in the matrix.

31. The method of any one of claims 1-30, wherein the macromolecular matrix has enhanced extrudability through a needle, wherein the needle comprises a gauge size of 27G, 28G, 29G, 30G, 31G or 32G.

32. The method of any one of claims 1-31, wherein the macromolecular matrix is stable for at least 6 months.

33. The method of any one of claims 1-32, wherein the macromolecular matrix is stable for about 6, about 9, about 12, about 15, about 18, about 21, about 24, about 27, about 30, about 33 or about 36 months or any amount of time in between a range defined by any two aforementioned values.

34. The method of any one of claims 1-33, wherein the macromolecular matrix is stable at a temperature of about 4° C., about 5° C., about 10° C., about 15° C., about 20° C., about 25° C. or any temperature in between a range defined by any two aforementioned values.

35. The method of any one of claims 1-34, wherein the macromolecular matrix is stable at about 4° C.

36. The method of any one of claims 1-35, wherein the macromolecular matrix is stable at about 25° C.

37. The method of any one of claims 1-36, wherein the macromolecular matrix has minimal degradation at about 3, about 6, about 9, about 12, about 15, about 18, about 21, about 24, about 27, about 30, about 33 or about 36 months or any amount of time in between a range defined by any two aforementioned values.

38. A method of preparing a macromolecular matrix, the method comprising:
dissolving hyaluronic acid in an aqueous solution to form an aqueous pre-reaction solution and preparing a second solution wherein the second solution comprises:
a) a water soluble carbodiimide; and
an N-hydroxysuccinimide or an N- hydroxysulfosuccinimide; or
b) 1,4-butanediol diglycidyl ether (BDDE) in a solution of sodium hyaluronate in the presence of sodium hydroxide; and
adding the second solution to the aqueous pre-reaction solution to form a crosslinking reaction mixture;
crosslinking the hyaluronic acid, wherein the crosslinking reaction mixture reacts by crosslinking the hyaluronic acid, thereby forming the crosslinked hyaluronic acid; and
providing collagen; and
physically mixing the collagen into the crosslinked hyaluronic acid, thereby producing a macromolecular matrix comprising crosslinked hyaluronic acid and collagen, wherein the collagen is mixed homogeneously throughout the crosslinked hyaluronic acid.

39. The method of claim 38, wherein the second solution comprises the water soluble carbodiimide and the N-hydroxysuccinimide or the N- hydroxysulfosuccinimide, wherein the hyaluronic acid is crosslinked with a naturally occurring amine, thereby forming the crosslinked hyaluronic acid.

40. The method of claim 38 or 39, wherein the second solution comprises the water soluble carbodiimide and the N-hydroxysuccinimide or the N- hydroxysulfosuccinimide and wherein the crosslinking is performed in the presence of MES.

41. The method of claim 38, wherein the second solution comprises the 1,4-butanediol diglycidyl ether (BDDE) in a solution of sodium hyaluronate in the presence of sodium hydroxide, and wherein the BDDE is used to cross-link the hyaluronic acid using epoxide chemistry.

42. The method of any one of claims 38-41, wherein the collagen is provided in a soluble state as a solution.

43. The method of any one of claims 38-42, wherein physically mixing step is performed in the presence of a buffer.

44. The method of claim 43, wherein the buffer comprises PBS.

45. The method of any one of claims 38-44, wherein the collagen is provided as a collagen solution, wherein the collagen solution comprises a pH of about 2.0, about 2.5, about 3.0, about 3.5, about 4.0, about 4.5, about 5.0, about 5.5, about 6.0, about 6.5, about 7.0 or any pH within a range defined by any two aforementioned values, and wherein the collagen is soluble at an acidic pH.

46. The method of any one of claims 38-45, wherein the collagen is provided as a solution in an acidic pH, wherein the collagen solution comprises a pH of about 2.0, about 2.5, about 3.0, about 3.5, about 4.0, about 4.5, about 5.0, about 5.5, or any pH within a range defined by any two aforementioned values, and wherein the collagen is soluble at the acidic pH.

47. The method of any one of claims 38-46, wherein the collagen is provided at an acidic to neutral pH.

48. The method of any one of claims 38-46, further comprising neutralizing the macromolecular matrix to a pH of about 7, after mixing the collagen homogeneously throughout the crosslinked hyaluronic acid.

49. The method of any one of claims 38-48, further comprising neutralizing the collagen to a pH of about 7, prior to physically mixing the collagen with the crosslinked hyaluronic acid, wherein neutralizing the collagen prior to physically mixing the collagen into the crosslinked hyaluronic acid causes the collagen to precipitate into fibrils or particles of collagen, wherein the fibrils or particles of collagen are further mixed into the crosslinked hyaluronic acid, wherein the fibrils or particles are mixed in homogeneously throughout the crosslinked hyaluronic acid.

50. The method of claim 49, wherein the collagen is provided as fibrillated collagen or collagen fibers.

51. The method of claim 50, wherein the collagen was prepared at a basic pH thereby producing the fibrillated collagen or collagen fibers.

52. The method of claim 50 or 51, wherein the collagen was prepared with at least one salt to obtain fibrillated collagen or collagen fibers.

53. The method of claim 52, wherein the at least one salt comprises a concentration of of about 20 mM, about 50 mM, about 100 mM, about 150 mM, about 200 mM, about 250 mM, about 300 mM, about 350 mM, about 400 mM, about 450 mM, or about 500 mM, or any concentration in between a range defined by any two aforementioned values.

54. The method of claim 52 or 53, wherein the at least one salt comprises an anion wherein the anion comprises H₂PO⁴⁻, SO₄²⁻, Cl⁻ or SCN⁻.

55. The method of any one of claims 52-54, wherein the at least one salt comprises NaCl, Na₂SO₄, or Li₂SO₄.

56. The method of any one of claims 38-55, wherein the hyaluronic acid comprises an average molecular weight of about 10,000 Daltons, about 20,000 Daltons, about 30,000 Daltons, 40,000 Daltons, about 50,000 Daltons, about 60,000 Daltons, about 70,000 Daltons, about 80,000 Daltons, about 90,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,100,000 Daltons, about 1,200,000 Daltons, about 1,300,000 Daltons, about 1,400,000 Daltons, about 1,500,000 Daltons, about 1,600,000 Daltons, about 1,700,000 Daltons, about 1,800,000 Daltons, about 1,900,000 Daltons, about 2,000,000 Daltons, about 2,100,000 Daltons, about 2,200,000 Daltons, about 2,300,000 Daltons, about 2,400,000 Daltons, about 2,500,000 Daltons, about 2,600,000 Daltons, about 2,700,000 Daltons, about 2,800,000 Daltons, about 2,900,000 Daltons, about 3,000,000 Daltons, about 3,100,000 Daltons, about 3,200,000 Daltons, about 3,300,000 Daltons, about 3,400,000 Daltons, about 3,500,000 Daltons, about 3,600,000 Daltons, about 3,700,000 Daltons, about 3,800,000 Daltons, about 3,900,000 Daltons, about 4,000,000 Daltons, about 4,100,000 Daltons, about 4,200,000 Daltons, about 4,300,000 Daltons, about 4,400,000 Daltons, about 4,500,000 Daltons, about 4,600,000 Daltons, about 4,700,000 Daltons, about 4,800,000 Daltons, about 4,900,000 Daltons, about 5,000,000 Daltons, about 5,100,000 Daltons, about 5,200,000 Daltons, about 5,300,000 Daltons, about 5,400,000 Daltons, about 5,500,000 Daltons, about 5,600,000 Daltons, about 5,700,000 Daltons, about 5,800,000 Daltons, about 5,900,000 Daltons, about 6,000,000 Daltons, about 6,100,000 Daltons, about 6,200,000 Daltons, about 6,300,000 Daltons, about 6,400,000 Daltons, about 6,500,000 Daltons, about 6,600,000 Daltons, about 6,700,000 Daltons, about 6,800,000 Daltons, about 6,900,000 Daltons, about 7,000,000 Daltons, about 7,100,000 Daltons, about 7,200,000 Daltons, about 7,300,000 Daltons, about 7,400,000 Daltons, about 7,500,000 Daltons, about 7,600,000 Daltons, about 7,700,000 Daltons, about 7,800,000 Daltons, about 7,900,000 Daltons, about 8,000,000 Daltons, about 8,100,000 Daltons, about 8,200,000 Daltons, about 8,300,000 Daltons, about 8,400,000 Daltons, about 8,500,000 Daltons, about 8,600,000 Daltons, about 8,700,000 Daltons, about 8,800,000 Daltons, about 8,900,000 Daltons, about 9,000,000 Daltons, about 9,100,000 Daltons, about 9,200,000 Daltons, about 9,300,000 Daltons, about 9,400,000 Daltons, about 9,500,000 Daltons, about 9,600,000 Daltons, about 9,700,000 Daltons, about 9,800,000 Daltons, about 9,900,000 Daltons or about 10,000,000 Daltons or any molecular weight in between a range defined by any two aforementioned values.

57. The method of any one of claims 38-56, wherein the hyaluronic acid comprises a mixture of hyaluronic acid components comprising at least two different molecular weights.

58. The method of any one of claims 38-57, wherein the dissolving step further comprises dissolving both hyaluronic acid and collagen in an aqueous solution to form an aqueous pre-reaction solution comprising both hyaluronic acid and collagen and wherein the crosslinking reaction reacts by crosslinking both the hyaluronic acid and collagen into an HA-Collagen conjugated gel, and wherein the mixing step comprises physically mixing the HA-Collagen conjugated gel with additional collagen, wherein the collagen is in a soluble state and wherein the collagen is in a solution, wherein the solution is in an acidic pH, thereby producing a macromolecular matrix comprising HA-Collagen conjugated gel with physically mixed in collagen.

59. The method of any one of claims 38-58, wherein collagen fibrils or collagen precipitates are formed after the mixing step with collagen, and wherein the collagen fibrils or collagen precipitates are mixed homogenously within the crosslinked hyaluronic acid.

60. The method of any one of claims 38-58, wherein collagen fibrils or collagen precipitates are formed during the mixing step with collagen, and wherein the collagen fibrils or collagen precipitates are mixed homogenously within the crosslinked hyaluronic acid

61. The method of any one of claims 1-60, wherein the method further comprises adjusting an osmolarity of the macromolecular matrix after the collagen is mixed throughout the crosslinked hyaluronic acid.

62. The method of any one of claims 1-61, wherein the mixing step is performed at room temperature.

63. The method of any one of claims 1-61, wherein the mixing step is performed at about 4° C.

64. The method of any one of claims 1-63, wherein the method is performed at about 4° C.

65. The method of any one of claims 1-64, wherein the method further comprises purifying the crosslinked hyaluronic acid or HA-Collagen conjugated gel, wherein the purifying is performed prior to the mixing step with the collagen.

66. The method of claim 65, wherein the purifying is performed using dialysis purification.

67. The method of claim 65 or 66, wherein the pH of the crosslinking reaction mixture is adjusted after the crosslinking is complete, wherein adjusting the pH is performed prior to the purifying step, and wherein the pH is adjust to about 7.0, about 7.2, about 7.4 about 7.6 or any pH in between a range defined by any two aforementioned values.

68. The method of any one of claims 65-67, wherein the purifying is performed at a range between about 2° C. and about 8° C.

69. The method of any one of claims 65-68, wherein the purifying is performed at about 2° C., about 4° C., about 6° C., or about 8° C., or any temperature in between a range defined by any two aforementioned values.

70. The method of any one of claims 65-69, wherein the method further comprises sterilizing the crosslinked hyaluronic acid or HA-Collagen conjugated gel prior to mixing with collagen, wherein the sterilizing step is performed after the purifying step.

71. The method of claim 70, wherein the sterilizing step comprises transferring the crosslinked hyaluronic acid or HA-Collagen conjugated gel into a container, for sterilization; and
sterilizing the crosslinked hyaluronic acid or HA-Collagen conjugated gel.

72. The method of claim 71, wherein the container is a syringe.

73. The method of any one of claims 70-72, wherein the sterilizing is performed by heat (dry heat, steam heat, moist heat sterilization) radiation (non-ionizing, UV), ionizing (particulate (beta rays, gamma rays, x-rays), electromagnetic (e-beam), filtration or terminal sterilization.

74. The method of any one of claims 1-73, wherein the method further comprises dialyzing the crosslinked macromolecular matrix or HA-Collagen conjugated gel, wherein dialysis is performed through a membrane having a molecular weight cutoff in a range between 1000 Daltons to about 100,000 Daltons, and wherein the dialyzing is performed prior to sterilization.

75. The method of claim 74, wherein the dialysis is performed in a buffer.

76. The method of claim 75, wherein the buffer comprises phosphate buffered saline or a sodium phosphate buffer.

77. The method of any one of claim 75 or 76, wherein the buffer further comprises NaCl, and/or KCl.

78. The method of any one of claims 38-75, wherein the method further comprises raising the pH of the crosslinked hyaluronic acid or HA-Collagen conjugated gel to a neutral pH after the crosslinking reaction is complete, wherein the raising the pH is performed prior to a sterilizing step.

79. The method of any one of claims 38-75, wherein the method further comprises raising the pH of the crosslinked hyaluronic acid or HA-Collagen conjugated gel to about 7.0, about 7.2 or about 7.4, or any pH in between a range defined by any two aforementioned values, after the crosslinking reaction is complete, wherein the raising the pH is performed prior to a sterilizing step.

80. The method of any one of claims 38-78, wherein the second solution comprises the water soluble carbodiimide and the N-hydroxysuccinimide or an N-hydroxysulfosuccinimide, and wherein the adding and crosslinking step is performed at a temperature between about 4° C. and about 22° C.

81. The method of claim 80, wherein the crosslinking is performed at about 22° C.

82. The method of claim 80, wherein the crosslinking is performed at about 4° C.

83. The method of any one of claims 38-78, wherein the second solution comprises the 1,4-butanediol diglycidyl ether (BDDE) in the solution of sodium hyaluronate in the presence of sodium hydroxide, and wherein the adding and crosslinking step is performed between about 45° C. and about 75° C.

84. The method of any one of claims 38-83, wherein the second solution comprises the 1,4-butanediol diglycidyl ether (BDDE) in the solution of sodium hyaluronate in the presence of sodium hydroxide, and wherein the adding and crosslinking step is performed at a temperature of about 45° C., about 50° C., about 55° C., about 60° C., about 65° C., about 70° C., about 75° C., or any temperature in between a range defined by any two aforementioned values.

85. The method of any one of claims 38-84, wherein the second solution comprises the 1,4-butanediol diglycidyl ether (BDDE) in the solution of sodium hyaluronate in the presence of sodium hydroxide, and wherein the adding and crosslinking step is performed at a temperature of about 50° C.

86. The method of any one of claims 1-85, wherein the method comprises a neutralizing step performed after forming the macromolecular matrix comprising the crosslinked hyaluronic acid and collagen or after forming the HA-Collagen conjugated gel with physically mixed in collagen, wherein the neutralizing step comprises adjusting the macromolecular matrix to a physiological pH and physiological salt concentration.

87. The method of claim 86, wherein the neutralizing comprises adding a basic solution or buffered solution after the mixing step under aseptic conditions to adjust the pH.

88. The method of claim 86 or 87, wherein adjusting the pH and physiological salt concentration causes precipitation of the collagen into fibrils or particles, wherein the collagen fibrils or particles are distributed homogeneously in the macromolecular matrix.

89. The method of any one of claims 38-88, wherein the method further comprises stopping the crosslinking step, wherein the stopping step comprises adding a base to the crosslinking reaction mixture to a pH of between about 8 and about 10 for at least 10 minutes and then adding an acidic solution to the crosslinking mixture until a pH of about 7 is reached.

90. The method of any one of claims 38-89, wherein the naturally occurring amine is from lysine.

91. The method of any one of claims 38-90, wherein the crosslinking reaction mixture comprises a pH between about 4.0 and about 10.0.

92. The method of claim 91, wherein the pH of the crosslinking reaction mixture is between about 4.0 and about 6.0.

93. The method of any one of claim 38-78 or 83-92, wherein the second solution comprises 1,4-butanediol diglycidyl ether (BDDE) in a solution of sodium hyaluronate in the presence of sodium hydroxide, and wherein the crosslinking is performed under alkaline conditions.

94. The method of any one of claims 38-93, wherein the pre-reaction solution comprises a salt, wherein the salt comprises a concentration of about 50 mM, about 75 mM, about 100 mM, about 125 mM, about 150 mM, about 175 mM, about 200 mM, about 225 mM, about 250 mM, about 275 mM, about 300 mM, 325 mM, about 350 mM, about 375 mM, about 400 mM, about 425 mM, about 450 mM, about 475 mM, about 500 mM or any concentration in a range defined by any two aforementioned values.

95. The method of any one of claim 38-81, 86-92 or 94, wherein the water soluble carbodiimide is 1-ethyl-3-(3-dimethylaminopropyl)carbodiimide and wherein the water soluble carbodiimide is at a concentration of about 20 mM to about 300 mM in the crosslinking reaction mixture.

96. The method of claim 95, wherein the concentration of the water soluble carbodiimide is 1-ethyl-3-(3-dimethylaminopropyl)carbodiimide is at a concentration of about 20 mM, about 40 mM, about 60 mM, about 80 mM, about 100 mM, about 120 mM, about 140 mM, about 160 mM, about 180 mM, about 200 mM, about 220 mM, about 240 mM, about 260 mM, about 280 mM, about 300 mM or any concentration in between a range defined by any two aforementioned values.

97. The method of any one of claim 38-81, 86-92 or 94-96, wherein the water soluble carbodiimide and hyaluronic acid is at a mole to mole ratio of water soluble carbodiimide:
hyaluronic acid repeat unit between about 0.3 to about 3.0.

98. The method of claim 97, wherein the water soluble carbodiimide and hyaluronic acid is at a mole to mole ratio of water soluble carbodiimide: hyaluronic acid repeat unit of about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9 or about 3.0 or any mole to mole ratio in between a range defined by any two aforementioned values.

99. The method of any one of claims 38-98, wherein the lysine and hyaluronic acid are at a mole:mole (lysine:HA repeat unit) ratio between about 0.01 to about 0.6.

100. The method of claim 99, wherein the lysine and hyaluronic acid are at a mole:mole (lysine:HA repeat unit) ratio of about 0.01, about 0.02, about 0.03, about 0.04, about 0.05, about 0.06, about 0.07, about 0.08, about 0.09, about 0.10, about 0.11, about 0.12, about 0.13, about 0.14, about 0.15, about 0.16, about 0.17, about 0.18, about 0.19, about 0.2, about 0.21, about 0.22, about 0.23, about 0.24, about 0.25, about 0.26, about 0.27, about 0.28, about 0.29, about 0.3, about 0.31, about 0.32, about 0.33, about 0.34, about 0.35, about 0.36, about 0.37, about 0.38, about 0.39, about 0.4, about 0.41, about 0.42, about 0.43, about 0.44, about 0.45, about 0.46, about 0.47, about 0.48, about 0.49, about 0.5, about 0.51, about 0.52, about 0.53, about 0.54, about 0.55, about 0.56, about 0.57, about 0.58, about 0.59, about 0.6 or any mole to mole ratio in between a range defined by any two aforementioned values.

101. The method of any one of claims 38-Error! Reference source not found., wherein the hyaluronic acid in the pre-reaction solution hydrates for at least about 60 minutes prior to the adding the second solution.

102. The method of any one of claims 38-101, wherein the crosslinking reaction mixture is performed for about 4 hours to about 24 hours.

103. The method of any one of claims 38-102, wherein the method further comprises adding lidocaine to the macromolecular matrix.

104. The method of claim 103, wherein the lidocaine is at a concentration in between a range of about 0.15% (w/w) to about 0.45% (w/w) in the matrix.

105. The method of claim 103 or 104, wherein the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w), about 0.39% (w/w), about 0.41% (w/w), about 0.43% (w/w), or about 0.45% (w/w) of the matrix or any concentration in between a range defined by any two aforementioned values.

106. The method of any one of claims 103-105, wherein the lidocaine is at a concentration in between a range of about 0.27% (w/w) to about 0.33% (w/w) in the matrix.

107. The method of any one of claims 38-106, wherein the matrix further comprises un-crosslinked HA.

108. The method of claim 107, wherein the un-crosslinked HA comprises a concentration of up to about 5% (w/w) within the matrix.

109. The method of claim 107-108, wherein the un-crosslinked HA comprises a concentration of about 0% (w/w), about 1% (w/w), about 2% (w/w), about 3% (w/w), about 4% (w/w), about 5% (w/w) in the matrix or any concentration in between a range defined by any two aforementioned values.

110. The method of any one of claims 107-109, wherein the un-crosslinked HA comprises a concentration of about 1% (w/w) in the matrix.

111. The method of any one of claims 107-109, wherein the un-crosslinked HA comprises a concentration of about 2% (w/w) in the matrix.

112. The method of any one of claims 107-109, wherein the un-crosslinked HA comprises a concentration of about 5% (w/w) in the matrix.

113. The method of any one of claims 38-112, wherein the macromolecular matrix has enhanced extrudability through a needle, wherein the needle comprises a gauge size of 27G, 28G, 29G, 30G, 31G or 32G.

114. The method of any one of claims 1-113, wherein the method further comprises concentrating the collagen prior to adding the collagen to the crosslinked HA.

115. A macromolecular matrix made by the method of any one of claims 1-114.

116. The macromolecular matrix of claim 115, wherein the macromolecular matrix comprises crosslinked hyaluronic acid, wherein the collagen is physically mixed with crosslinked hyaluronic acid, and wherein the collagen is homogeneous throughout the macromolecular complex.

117. The macromolecular matrix of claim 115, wherein the macromolecular matrix comprises HA-Collagen conjugated gel, wherein the HA-Collagen conjugated gel also comprises physically mixed in collagen that is not crosslinked to the HA-Collagen conjugated gel.

118. A macromolecular matrix comprising:
hyaluronic acid, wherein the hyaluronic acid is crosslinked; and
collagen; wherein the collagen is physically mixed with the crosslinked hyaluronic acid.

119. A macromolecular matrix comprising:
an HA-Collagen conjugated gel, wherein the HA-Collagen conjugated gel comprises hyaluronic acid crosslinked with collagen; and
physically mixed in collagen, wherein the physically mixed in collagen is not crosslinked to the HA-Collagen conjugated gel, and wherein the physically mixed in collagen is mixed homogeneously within the HA-Collagen conjugated gel.

120. The macromolecular matrix of any one of claims 115-119, wherein the hyaluronic acid is crosslinked with a naturally occurring amine.

121. The macromolecular matrix of claim 120, wherein the naturally occurring amine is from lysine.

122. The macromolecular matrix of any one of claims 115-121, wherein the collagen is from bovine collagen, marine collagen, human collagen or porcine collagen.

123. The macromolecular matrix of any one of claims 115-122, wherein the collagen is recombinant human collagen.

124. The macromolecular matrix of any one of claims 115-123, wherein the macromolecular matrix comprises an elastic modulus (G′) of about 100 Pa, about 200 Pa, about 300 Pa, about 400 Pa, about 500 Pa, about 600 Pa, about 700 Pa, about 800 Pa, about 900 Pa, about 1000 Pa, about 1100 Pa, about 1200 Pa, about 1300 Pa, about 1400 Pa, about 1500 Pa, about 1600 Pa, about 1700 Pa, about 1800 Pa, about 1900 Pa, about 2000 Pa, about 2100 Pa, about 2200 Pa, about 2300 Pa, about 2400 Pa, about 2500 Pa, about 2600 Pa, about 2700 Pa, about 2800 Pa, about 2900 Pa, or about 3000 Pa or any value in between a range defined by any two aforementioned values.

125. The macromolecular matrix of any one of claims 115-124, wherein the macromolecular matrix comprises a compression force value of about 10 gmf, about 20 gmf, about 30 gmf, about 40 gmf, about 50 gmf, about 60 gmf, about 70 gmf, about 80 gmf, about 90 gmf, about 100 gmf, about 110 gmf, about 120 gmf, about 130 gmf, about 140 gmf, about 150 gmf, about 160 gmf, about 170 gmf, about 180 gmf, about 190 gmf, about 200 gmf, about 210 gmf, about 220 gmf, about 230 gmf, about 240 gmf, about 250 gmf, about 260 gmf, about 270 gmf, about 280 gmf, about 290 gmf, about 300 gmf, about 310 gmf, about 320 gmf, about 330 gmf, about 340 gmf, about 350 gmf, about 360 gmf, about 370 gmf, about 380 gmf, about 390 gmf, about 400 gmf, about 410 gmf, about 420 gmf, about 430 gmf, about 440 gmf, about 450 gmf, about 460 gmf, about 470 gmf, about 480 gmf, about 490 gmf, about 500 gmf, about 510 gmf, about 520 gmf, about 530 gmf, about 540 gmf, about 550 gmf, about 560 gmf, about 570 gmf, about 580 gmf, about 590 gmf or about 600 gmf or any compression force value in between a range defined by any two aforementioned values.

126. The macromolecular matrix of any one of claims 115-125, wherein the hyaluronic acid is at a concentration of about 2 mg/ml, about 4 mg/ml, about 6 mg/ml, about 8 mg/ml, about 10 mg/ml, about 12 mg/ml, about 14 mg/ml, about 16 mg/ml, about 18 mg/ml, about 20 mg/ml, about 22 mg/ml, about 24 mg/ml, about 26 mg/ml about 28 mg/ml, about 30 mg/ml, 32 mg/ml, about 34 mg/ml or about 36 mg/ml or any concentration in between a range defined by any two aforementioned values.

127. The macromolecular matrix of any one of claims 115-126, having a weight ratio of hyaluronic acid to collagen at about 20:3, about 24:2.3, about 24:10, about 24:12, about 24:4, about 24:6, about 28:6 or about 28:11.

128. The macromolecular matrix of any one of claims 115-127, wherein the crosslinked hyaluronic acid comprises hyaluronic acid components comprising an average molecular weight of about 10,000 Daltons, about 20,000 Daltons, about 30,000 Daltons, 40,000 Daltons, about 50,000 Daltons, about 60,000 Daltons, about 70,000 Daltons, about 80,000 Daltons, about 100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,100,000 Daltons, about 1,200,000 Daltons, about 1,300,000 Daltons, about 1,400,000 Daltons, about 1,500,000 Daltons, about 1,600,000 Daltons, about 1,700,000 Daltons, about 1,800,000 Daltons, about 1,900,000 Daltons, about 2,000,000 Daltons, about 2,100,000 Daltons, about 2,200,000 Daltons, about 2,300,000 Daltons, about 2,400,000 Daltons, about 2,500,000 Daltons, about 2,600,000 Daltons, about 2,700,000 Daltons, about 2,800,000 Daltons, about 2,900,000 Daltons, about 3,000,000 Daltons, about 3,100,000 Daltons, about 3,200,000 Daltons, about 3,300,000 Daltons, about 3,400,000 Daltons, about 3,500,000 Daltons, about 3,600,000 Daltons, about 3,700,000 Daltons, about 3,800,000 Daltons, about 3,900,000 Daltons, about 4,000,000 Daltons, about 4,100,000 Daltons, about 4,200,000 Daltons, about 4,300,000 Daltons, about 4,400,000 Daltons, about 4,500,000 Daltons, about 4,600,000 Daltons, about 4,700,000 Daltons, about 4,800,000 Daltons, about 4,900,000 Daltons, about 5,000,000 Daltons, about 5,100,000 Daltons, about 5,200,000 Daltons, about 5,300,000 Daltons, about 5,400,000 Daltons, about 5,500,000 Daltons, about 5,600,000 Daltons, about 5,700,000 Daltons, about 5,800,000 Daltons, about 5,900,000 Daltons, about 6,000,000 Daltons, about 6,100,000 Daltons, about 6,200,000 Daltons, about 6,300,000 Daltons, about 6,400,000 Daltons, about 6,500,000 Daltons, about 6,600,000 Daltons, about 6,700,000 Daltons, about 6,800,000 Daltons, about 6,900,000 Daltons, about 7,000,000 Daltons, about 7,100,000 Daltons, about 7,200,000 Daltons, about 7,300,000 Daltons, about 7,400,000 Daltons, about 7,500,000 Daltons, about 7,600,000 Daltons, about 7,700,000 Daltons, about 7,800,000 Daltons, about 7,900,000 Daltons, about 8,000,000 Daltons, about 8,100,000 Daltons, about 8,200,000 Daltons, about 8,300,000 Daltons, about 8,400,000 Daltons, about 8,500,000 Daltons, about 8,600,000 Daltons, about 8,700,000 Daltons, about 8,800,000 Daltons, about 8,900,000 Daltons, about 9,000,000 Daltons, about 9,100,000 Daltons, about 9,200,000 Daltons, about 9,300,000 Daltons, about 9,400,000 Daltons, about 9,500,000 Daltons, about 9,600,000 Daltons, about 9,700,000 Daltons, about 9,800,000 Daltons, about 9,900,000 Daltons or about 10,000,000 Daltons or any weight in between a range defined by any two aforementioned values.

129. The macromolecular matrix of claim 128, wherein the hyaluronic acid components comprises a mixture of hyaluronic acid components comprising at least two different molecular weights.

130. The macromolecular matrix of any one of claims 115-129, wherein the hyaluronic acid comprises a mixture of hyaluronic acids, wherein the mixture comprises a 50:50 blend of HA comprising two different molecular weights.

131. The macromolecular matrix of any one of claims 115-130, wherein the collagen comprises Type I collagen and/or Type III collagen.

132. The macromolecular matrix of any one of claims 115-131, wherein the collagen is at a concentration of about 1 mg/ml, about 2 mg/ml, about 3 mg/ml, about 4 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml or about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml or any concentration in between a range defined by any two aforementioned values.

133. The macromolecular matrix of any one of claims 115-132, wherein the macromolecular matrix comprises an osmolality between about 250 mOsm/kg and about 390 mOsm/kg.

134. The macromolecular matrix of any one of claims 115-133, wherein the macromolecular matrix comprises an osmolality between about 250 mOsm/kg, about 275 mOsm/kg, about 300 mOsm/kg, about 325 mOsm/kg, or about 390 mOsm/kg or any osmolality in between a range defined by any two aforementioned values.

135. The macromolecular matrix of any one of claims 115-134, wherein the hyaluronic acid is linear.

136. The macromolecular matrix of any one of claims 115-135, wherein the macromolecular matrix comprises a viscous modulus (G″) of about 10 Pa, about 20 Pa, about 30 Pa, about 40 Pa, about 50 Pa, about 60 Pa, about 70 Pa, about 80 Pa, about 90 Pa, about 100 Pa, about 200 Pa, about 300 Pa, about 400 Pa, about 500 Pa, about 600 Pa, about 700 Pa, about 800 Pa, about 900 Pa or about 1000 Pa or any viscous modulus (G″) in between a range defined by any two aforementioned values.

137. The macromolecular matrix of any one of claims 115-136, wherein the macromolecular matrix comprises a tan delta (G″/G′) of about 0.01, about 0.02, about 0.03, about 0.04, about 0.05, about 0.06, about 0.07, about 0.08, about 0.09, about 0.1, about 0.12, about 0.14, about 0.16, about 0.18, about 0.20, about 0.22, about 0.24, about 0.26, about 0.28, about 0.30, about 0.32, about 0.34, about 0.36, about 0.38, about 0.40, about 0.42, about 0.44, about 0.46, about 0.48, about 0.50 or any tan delta (G″/G′) in between a range defined by any two aforementioned values.

138. The macromolecular matrix of any one of claims 115-137, wherein the macromolecular matrix comprises a pH of about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0 or any pH in a range defined by any two aforementioned values

139. The macromolecular matrix of any one of claims 115-138, wherein the macromolecular matrix further comprises lidocaine.

140. The macromolecular matrix of claim 139, wherein the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w), about 0.39% (w/w), about 0.41% (w/w), about 0.43% (w/w), or about 0.45% (w/w) of the matrix or any concentration in between a range defined by any two aforementioned values.

141. The macromolecular matrix of any one of claim 139 or 140, wherein the lidocaine is at a concentration in between a range of about 0.27% (w/w) to about 0.33% (w/w) in the matrix.

142. The macromolecular matrix of any one of claims 115-141, wherein the matrix further comprises un-crosslinked HA.

143. The macromolecular matrix of claim 142, wherein the un-crosslinked HA comprises a concentration of up to about 5% (w/w) within the matrix.

144. The macromolecular matrix of claim 142-143, wherein the un-crosslinked HA comprises a concentration of about 0% (w/w), about 1% (w/w), about 2% (w/w), about 3% (w/w), about 4% (w/w), about 5% (w/w) in the matrix or any concentration in between a range defined by any two aforementioned values.

145. The macromolecular matrix of any one of claims 142-144, wherein the un-crosslinked HA comprises a concentration of about 1% (w/w) in the matrix.

146. The macromolecular matrix of any one of claims 142-144, wherein the un-crosslinked HA comprises a concentration of about 2% (w/w) in the matrix.

147. The macromolecular matrix of any one of claims 142-144, wherein the un-crosslinked HA comprises a concentration of about 5% (w/w) in the matrix.

148. The macromolecular matrix of any one of claims 115-147, wherein the macromolecular matrix has enhanced extrudability through a needle, wherein the needle comprises a gauge size of 27G, 28G, 29G, 30G, 31G or 32G.

149. A method of improving an aesthetic quality of an anatomic feature of a human being comprising:
injecting a composition into a tissue of the human being to thereby improve the aesthetic quality of the anatomic feature;
wherein the composition comprises the macromolecular matrix prepared by the method of any one of claims 1-102 or the macromolecular matrix of any one of claims 115-139.

150. A method of improving an aesthetic quality of an anatomic feature of a human being comprising:
injecting a composition into a tissue of the human being to thereby improve the aesthetic quality of the anatomic feature;
wherein the composition comprises a macromolecular matrix comprising:
crosslinked hyaluronic acid;
lysine; and
collagen;
wherein the collagen is physically mixed into the crosslinked hyaluronic acid.

151. The method of claim 150, wherein the composition further comprises lidocaine.

152. The method of claim 151, wherein the lidocaine is at a concentration in between a range of 0.15% (w/w) to 0.45% (w/w) in the matrix.

153. The method of claim 151 or 152, wherein the lidocaine is at a concentration of about 0.15% (w/w), about 0.17% (w/w), about 0.19% (w/w), about 0.21% (w/w), about 0.23% (w/w), about 0.25% (w/w), about 0.27% (w/w), about 0.29% (w/w), about 0.31% (w/w), about 0.33% (w/w), about 0.35% (w/w), about 0.37% (w/w), about 0.39% (w/w), about 0.41% (w/w), about 0.43% (w/w), or about 0.45% (w/w) of the matrix or any concentration in between a range defined by any two aforementioned values.

154. The method of any one of claims 151-153, wherein the lidocaine is at a concentration in between a range of about 0.27% (w/w) to about 0.33% (w/w) in the matrix.

155. The method of any one of claims 150-154, wherein the composition further comprises un-crosslinked HA.

156. The method of claim 155, wherein the un-crosslinked HA comprises a concentration of up to about 5% (w/w) within the matrix.

157. The method of claim 155 or 156, wherein the un-crosslinked HA comprises a concentration of about 0% (w/w), about 1% (w/w), about 2% (w/w), about 3% (w/w), about 4% (w/w), about 5% (w/w) in the matrix, or any concentration in between a range defined by any two aforementioned values.

158. The method of any one of claims 155-157, wherein the un-crosslinked HA comprises a concentration of about 1% (w/w) in the matrix.

159. The method of any one of claims 155-157, wherein the un-crosslinked HA comprises a concentration of about 2% (w/w) in the matrix.

160. The method of any one of claims 155-157, wherein the un-crosslinked HA comprises a concentration of about 5% (w/w) in the matrix.

161. The method of any one of claims 150-160, wherein the crosslinked hyaluronic acid comprises hyaluronic acid components comprising an average molecular weight of about 10,000 Daltons, about 20,000 Daltons, about 30,000 Daltons, 40,000 Daltons, about 50,000 Daltons, about 60,000 Daltons, about 70,000 Daltons, about 80,000 Daltons, about100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,100,000 Daltons, about 1,200,000 Daltons, about 1,300,000 Daltons, about 1,400,000 Daltons, about 1,500,000 Daltons, about 1,600,000 Daltons, about 1,700,000 Daltons, about 1,800,000 Daltons, about 1,900,000 Daltons, about 2,000,000 Daltons, about 2,100,000 Daltons, about 2,200,000 Daltons, about 2,300,000 Daltons, about 2,400,000 Daltons, about 2,500,000 Daltons, about 2,600,000 Daltons, about 2,700,000 Daltons, about 2,800,000 Daltons, about 2,900,000 Daltons, about 3,000,000 Daltons, about 3,100,000 Daltons, about 3,200,000 Daltons, about 3,300,000 Daltons, about 3,400,000 Daltons, about 3,500,000 Daltons, about 3,600,000 Daltons, about 3,700,000 Daltons, about 3,800,000 Daltons, about 3,900,000 Daltons, about 4,000,000 Daltons, about 4,100,000 Daltons, about 4,200,000 Daltons, about 4,300,000 Daltons, about 4,400,000 Daltons, about 4,500,000 Daltons, about 4,600,000 Daltons, about 4,700,000 Daltons, about 4,800,000 Daltons, about 4,900,000 Daltons, about 5,000,000 Daltons, about 5,100,000 Daltons, about 5,200,000 Daltons, about 5,300,000 Daltons, about 5,400,000 Daltons, about 5,500,000 Daltons, about 5,600,000 Daltons, about 5,700,000 Daltons, about 5,800,000 Daltons, about 5,900,000 Daltons, about 6,000,000 Daltons, about 6,100,000 Daltons, about 6,200,000 Daltons, about 6,300,000 Daltons, about 6,400,000 Daltons, about 6,500,000 Daltons, about 6,600,000 Daltons, about 6,700,000 Daltons, about 6,800,000 Daltons, about 6,900,000 Daltons, about 7,000,000 Daltons, about 7,100,000 Daltons, about 7,200,000 Daltons, about 7,300,000 Daltons, about 7,400,000 Daltons, about 7,500,000 Daltons, about 7,600,000 Daltons, about 7,700,000 Daltons, about 7,800,000 Daltons, about 7,900,000 Daltons, about 8,000,000 Daltons, about 8,100,000 Daltons, about 8,200,000 Daltons, about 8,300,000 Daltons, about 8,400,000 Daltons, about 8,500,000 Daltons, about 8,600,000 Daltons, about 8,700,000 Daltons, about 8,800,000 Daltons, about 8,900,000 Daltons, about 9,000,000 Daltons, about 9,100,000 Daltons, about 9,200,000 Daltons, about 9,300,000 Daltons, about 9,400,000 Daltons, about 9,500,000 Daltons, about 9,600,000 Daltons, about 9,700,000 Daltons, about 9,800,000 Daltons, about 9,900,000 Daltons or about 10,000,000 Daltons or any weight in between a range defined by any two aforementioned values.

162. The method of any one of claims 150-161, wherein the hyaluronic acid of the crosslinked hyaluronic acid comprises a mixture of hyaluronic acid components comprising at least two different molecular weights.

163. The method of any one of claims 150-162, wherein the collagen comprises collagen type I and/or collagen type III.

164. The method of any one of claims 150-163, wherein the method does not cause a bluish discoloration at a site of injection.

165. The method of any one of claims 150-164, wherein the composition has enhanced extrudability through a needle, wherein the needle comprises a gauge size of 27G, 28G, 29G, 30G, 31G or 32G.

166. A method of improving the appearance of a human being by injecting a composition into the tissue of a human being whereby the composition promotes cell infiltration and collagen deposition into the composition from the surrounding tissue into which it was injected, the method comprising:
injecting a composition into a tissue of the human being to thereby improve the aesthetic quality of the anatomic feature;
wherein the composition comprises a crosslinked macromolecular matrix comprising:
hyaluronic acid;
lysine; and
collagen;
wherein the hyaluronic acid comprises crosslinked hyaluronic acid that is physically mixed with the collagen; and
wherein the tissue injected by the composition is shown to have tissue integration.

167. The method of claim 166, wherein the composition further comprises lidocaine.

168. The method of claim 166 or 167, wherein the hyaluronic acid component has an average molecular weight of about 10,000 Daltons, about 20,000 Daltons, about 30,000 Daltons, 40,000 Daltons, about 50,000 Daltons, about 60,000 Daltons, about 70,000 Daltons, about 80,000 Daltons, about100,000 Daltons, about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,100,000 Daltons, about 1,200,000 Daltons, about 1,300,000 Daltons, about 1,400,000 Daltons, about 1,500,000 Daltons, about 1,600,000 Daltons, about 1,700,000 Daltons, about 1,800,000 Daltons, about 1,900,000 Daltons, about 2,000,000 Daltons, about 2,100,000 Daltons, about 2,200,000 Daltons, about 2,300,000 Daltons, about 2,400,000 Daltons, about 2,500,000 Daltons, about 2,600,000 Daltons, about 2,700,000 Daltons, about 2,800,000 Daltons, about 2,900,000 Daltons, about 3,000,000 Daltons, about 3,100,000 Daltons, about 3,200,000 Daltons, about 3,300,000 Daltons, about 3,400,000 Daltons, about 3,500,000 Daltons, about 3,600,000 Daltons, about 3,700,000 Daltons, about 3,800,000 Daltons, about 3,900,000 Daltons, about 4,000,000 Daltons, about 4,100,000 Daltons, about 4,200,000 Daltons, about 4,300,000 Daltons, about 4,400,000 Daltons, about 4,500,000 Daltons, about 4,600,000 Daltons, about 4,700,000 Daltons, about 4,800,000 Daltons, about 4,900,000 Daltons, about 5,000,000 Daltons, about 5,100,000 Daltons, about 5,200,000 Daltons, about 5,300,000 Daltons, about 5,400,000 Daltons, about 5,500,000 Daltons, about 5,600,000 Daltons, about 5,700,000 Daltons, about 5,800,000 Daltons, about 5,900,000 Daltons, about 6,000,000 Daltons, about 6,100,000 Daltons, about 6,200,000 Daltons, about 6,300,000 Daltons, about 6,400,000 Daltons, about 6,500,000 Daltons, about 6,600,000 Daltons, about 6,700,000 Daltons, about 6,800,000 Daltons, about 6,900,000 Daltons, about 7,000,000 Daltons, about 7,100,000 Daltons, about 7,200,000 Daltons, about 7,300,000 Daltons, about 7,400,000 Daltons, about 7,500,000 Daltons, about 7,600,000 Daltons, about 7,700,000 Daltons, about 7,800,000 Daltons, about 7,900,000 Daltons, about 8,000,000 Daltons, about 8,100,000 Daltons, about 8,200,000 Daltons, about 8,300,000 Daltons, about 8,400,000 Daltons, about 8,500,000 Daltons, about 8,600,000 Daltons, about 8,700,000 Daltons, about 8,800,000 Daltons, about 8,900,000 Daltons, about 9,000,000 Daltons, about 9,100,000 Daltons, about 9,200,000 Daltons, about 9,300,000 Daltons, about 9,400,000 Daltons, about 9,500,000 Daltons, about 9,600,000 Daltons, about 9,700,000 Daltons, about 9,800,000 Daltons, about 9,900,000 Daltons or about 10,000,000 Daltons or any weight in between a range defined by any two aforementioned values.

169. The method of any one of claims 166-168, wherein the hyaluronic acid of the crosslinked hyaluronic acid comprises a mixture of hyaluronic acid components comprising at least two different molecular weights.

170. The method of any one of claims 166-169, wherein the collagen comprises collagen type I and/or collagen type III.

171. The method of any one of claims 166-170, wherein the tissue injected by the composition is shown to have tissue integration into the composition and collagen deposition within the composition by infiltrating cells from surrounding tissue.

172. The method of any one of claims 166-171, wherein the tissue injected by the composition is shown to have tissue integration into the composition and collagen deposition within the composition after injecting the composition.

173. The method of any one of claims 166-172, wherein the composition has enhanced extrudability through a needle, wherein the needle comprises a gauge size of 27G, 28G, 29G, 30G, 31G or 32G.
